SEQUENCE LISTING (39 sequences)

SEQ ID NO: 1 - DNA, 129 bp, Artificial Sequence
  Description of Artificial Sequence Synthetic polynucleotide
gtatcaaggttacaagacaggtttaaggagaccaatagaaactgggcttgtcgagacagagaagactcttgcgtttctgataggcacctattggtcttactgacatccactttgcctttctctccacag

SEQ ID NO: 2 - DNA, 217 bp, Artificial Sequence
  Description of Artificial Sequence Synthetic polynucleotide
gtatcaaggttacaagacaggtttaaggagaccaatagaaactgggcttgtcgagacagatctcttacacaggctgaccgatttctcctggtgttcagagtctgtttttgtctagcaccatttgaaatcggttatgatgtagggggaagaagactcttgcgtttctgataggcacctattggtcttactgacatccactttgcctttctctccacag

SEQ ID NO: 3 - DNA, 88 bp, Artificial Sequence
  Description of Artificial Sequence Synthetic oligonucleotide
atctcttacacaggctgaccgatttctcctggtgttcagagtctgtttttgtctagcaccatttgaaatcggttatgatgtaggggga

SEQ ID NO: 4 - DNA, 722 bp, Homo sapiens
agacagcctgggaggggagaaggagttggagctcaagttggagacagcgaggagaaacctgccatagccagggtgtgtctttgatcctcttcaggaggtgaggagaagccagaggtccttggtgtgccctcagaaatctgcctgcagttctcaccaagccgctgtgaaaatggggataaacacccgggagctgtttctcaacttcactattgtcttgattacggttattcttatgtggctccttgtgaggtcctatcagtactgagaggccatgccatggtcctgggattgactgagatgctccggagctgcctgctctatgccctgagaccccactgctgtcattgtcacaggatgccattctccatccgagggcacctgtgacctgcactcacaatatctgctatgctgtagtgctaggattgattatgtgttctccaaagatgctgctcccaagggctgccaagtgtttgccagggaacggtagatttattccccaactcttaactgaaaatgtgttagacaagccacaaagttaaaattaaactggattcatgatgatgtaggattgttacaagcccctgatctgtctcaccacacatcccttcaacccacacggtctgcaaccaaactctaattcaacctgccagaaggaatgttagaggaagtctttgtcagcccttatagctatcatgtgaataaagttaagtcaacttcaaaaa

SEQ ID NO: 5 - protein, 31 aa, Homo sapiens
MGINTRELFLNFTIVLITVILMWLLVRSYGY

SEQ ID NO: 6 - DNA, 22 bp, Artificial Sequence
  Description of Artificial Sequence Synthetic oligonucleotide
accgatttcaaatggtgctaga

SEQ ID NO: 7 - DNA, 24 bp, Artificial Sequence
  Description of Artificial Sequence Synthetic oligonucleotide
tctagcaccatttgaaatcggtta

SEQ ID NO: 8 - DNA, 15 bp, Artificial Sequence
  Description of Artificial Sequence Synthetic oligonucleotide
gtgaagccacagatg

SEQ ID NO: 9 - DNA, 206 bp, Artificial Sequence
  Description of Artificial Sequence Synthetic polynucleotide
cagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgg

SEQ ID NO: 10 - DNA, 358 bp, Artificial Sequence
  Description of Artificial Sequence Synthetic polynucleotide
gctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 11 - protein, 9 aa, Unknown
  Description of Unknown “LAGLIDADG” family peptide motif sequence
LAGLIDADG

SEQ ID NO: 12 - DNA, 157 bp, Artificial Sequence
  Description of Artificial Sequence Synthetic polynucleotide
tcgacttcttaacccaacagaaggctcgagaaggtatattgctgttgacagtgagcgtaaccgatttcaaatggtgctatagtgaagccacagatgtatagcaccatttgaaatcggttatgcctactgcctcggacttcaaggggctagaattcga

SEQ ID NO: 13 - DNA, 159 bp, Artificial Sequence
  Description of Artificial Sequence Synthetic polynucleotide
tcgacttcttaacccaacagaaggctcgagaaggtatattgctgttgacagtgagcgaacttcactattgtcttgattactagtgaagccacagatgtagtaatcaagacaatagtgaagtttgcctactgcctcggacttcaaggggctagaattcga

SEQ ID NO: 14 - DNA, 159 bp, Artificial Sequence
  Description of Artificial Sequence Synthetic polynucleotide
tcgacttcttaacccaacagaaggctcgagaaggtatattgctgttgacagtgagcgaacacccgggagctgtttctcaatagtgaagccacagatgtattgagaaacagctcccgggtgtttgcctactgcctcggacttcaaggggctagaattcga

SEQ ID NO: 15 - DNA, 153 bp, Artificial Sequence
  Description of Artificial Sequence Synthetic polynucleotide
ggttaacccaacagaaggctaaagaaggtatattgctgttgacagtgagcgactagcaccatttgaaatcggttactgtgaagccacagatgggtaaccgattaaatggtgctagctgcctactgcctcggacttcaaggggctactttagga

SEQ ID NO: 16 - DNA, 204 bp, Artificial Sequence
  Description of Artificial Sequence Synthetic polynucleotide
ccaccagaaaggatgccgttgaccgacacagtgactgacaggctgccctggcgaaccgatttcaaatggtgcataccgtctattctaaaggtagcaccatttgaaatcggttaggatggcagccatcttaccttccatcagaggagcctcaccgtacccaggaagaaagaaggtgaaagaggaatgtgaaacaggtggctggga

SEQ ID NO: 17 - DNA, 133 bp, Artificial Sequence
  Description of Artificial Sequence Synthetic polynucleotide
cctggaggcttgctgaaggctgtatgctgtagcaccatttgaaatcggttattttggcctctgactgatgaccgctggaatggtgctacaggacacaaggcctgttactagcactcacatggaacaaatggcc

SEQ ID NO: 18 - DNA, 135 bp, Artificial Sequence
  Description of Artificial Sequence Synthetic polynucleotide
cctggaggcttgctgaaggctgtatgctgtagcaccatttgaaatcggttagttttggccactgactgactaaccgatcaaatggtgctacaggacacaaggcctgttactagcactcacatggaacaaatggcc

SEQ ID NO: 19 - DNA, 133 bp, Artificial Sequence
  Description of Artificial Sequence Synthetic polynucleotide
cctggaggcttgctgaaggctgtatgctggtgatgaggacaactgtgaaggttttggccactgactgaccttcacaggtcctcatcaccaggacacaaggcctgttactagcactcacatggaacaaatggcc

SEQ ID NO: 20 - DNA, 137 bp, Artificial Sequence
  Description of Artificial Sequence Synthetic polynucleotide
cctggaggcttgctgaaggctgtatgctggtgatgaggacaactgtgaagttgttttggccactgactgacaacttcacttgtcctcatcaccaggacacaaggcctgttactagcactcacatggaacaaatggcc

SEQ ID NO: 21 - DNA, 137 bp, Artificial Sequence
  Description of Artificial Sequence Synthetic polynucleotide
cctggaggcttgctgaaggctgtatgctggtaatcaagacaatagtgaagttgttttggccactgactgacaacttcacttgtcttgattaccaggacacaaggcctgttactagcactcacatggaacaaatggcc

SEQ ID NO: 22 - DNA, 139 bp, Artificial Sequence
  Description of Artificial Sequence Synthetic polynucleotide
cctggaggcttgctgaaggctgtatgctgttgagaaacagctcccgggtgttgttttggccactgactgacaacacccgggagctgtttctcaacaggacacaaggcctgttactagcactcacatggaacaaatggcc

SEQ ID NO: 23 - DNA, 215 bp, Artificial Sequence
  Description of Artificial Sequence Synthetic polynucleotide
ggacaggagagatgctgcaagcccaagaagctctctgctcagcctgtcacaacctactgactgccagggcacttgggaatggcaaggtagcaccatttgaaatcggttacgatttcaaatggtgctgtcttgctatacccagaaaacgtgccaggaagagaactcaggaccctgaagcagactactggaagggagactccagctcaaacaaggca

SEQ ID NO: 24 - DNA, 74 bp, Artificial Sequence
  Description of Artificial Sequence Synthetic oligonucleotide
gatcgtaaccgatttcaaatggtgctagccctgacccagctagcaccatttgaaatcggttattttttgaagct

SEQ ID NO: 25 - DNA, 65 bp, Artificial Sequence
  Description of Artificial Sequence Synthetic oligonucleotide
gatcgacttcacagttgtcctcatcactcgagtgatgaggacaactgtgaagcttttttgaagct

SEQ ID NO: 26 - DNA, 67 bp, Artificial Sequence
  Description of Artificial Sequence Synthetic oligonucleotide
gatcgacttcacagttgtcctcatcactcaagaggtgatgaggacaactgtgaagttttttgaagct

SEQ ID NO: 27 - DNA, 61 bp, Artificial Sequence
  Description of Artificial Sequence Synthetic oligonucleotide
taggaagtcttttaataaaagatccttattttcattggatctgtgtgttggttttttgtgt

SEQ ID NO: 28 - DNA, 698 bp, Artificial Sequence
  Description of Artificial Sequence Synthetic polynucleotide
tcagatccaagctttagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaaaactagtgtatcaaggttacaagacaggtttaaggagaccaatagaaactgggcttgtcgagacagatctcttacacaggcacttcacagttgtcctcatcacagagtctgtttttgtcagtgatgaggacaactgtgaagtgatgtagggggaagaagactcttgcgtttctgataggcacctattggtcttactgacatccactttgcctttctctccacaggcctctagaacc

SEQ ID NO: 29 - DNA, 698 bp, Artificial Sequence
  Description of Artificial Sequence Synthetic polynucleotide
tcagatccaagctttagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaaaactagtgtatcaaggttacaagacaggtttaaggagaccaatagaaactgggcttgtcgagacagatctcttacacaggcagtgatgaggacaactgtgaagagagtctgtttttgtcacttcacagttgtcctcatcactgatgtagggggaagaagactcttgcgtttctgataggcacctattggtcttactgacatccactttgcctttctctccacaggcctctagaacc

SEQ ID NO: 30 - DNA, 685 bp, Artificial Sequence
  Description of Artificial Sequence Synthetic polynucleotide
tcagatccaagctttagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaaaactagtgtatcaaggttacaagacaggtttaaggagaccaatagaaactgggcttgtcgagacagatctcttacacaggcacttcacagttgtcctcatcactcgagtgatgaggacaactgtgaagtgatgtagggggaagaagactcttgcgtttctgataggcacctattggtcttactgacatccactttgcctttctctccacaggcctctagaacc

SEQ ID NO: 31 - DNA, 685 bp, Artificial Sequence
  Description of Artificial Sequence Synthetic polynucleotide
tcagatccaagctttagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccaaaactagtgtatcaaggttacaagacaggtttaaggagaccaatagaaactgggcttgtcgagacagatctcttacacaggcgaagtgtcaacaggagtagtgagctcactactcctgttgacacttcatgatgtagggggaagaagactcttgcgtttctgataggcacctattggtcttactgacatccactttgcctttctctccacaggcctctagaacc

SEQ ID NO: 32 - DNA, 76 bp, Homo sapiens
ggagctgtttctcaacttcactattgtcttgattacggttattcttatgtggctccttgtgaggtcctatcagtac

SEQ ID NO: 33 - DNA, 76 bp, Mus sp.
ggagctgtttatcaacttcacagttgtcctcatcaccgttctccttatgtggctcctcgtgaggtcctaccaatac

SEQ ID NO: 34 - DNA, 96 bp, Mus sp.
atggagaggtctactcaggagctgtttatcaacttcacagttgtcctcatcaccgttctccttatgtggctcctcgtgaggtcctaccaatactga

SEQ ID NO: 35 - DNA, 96 bp, Homo sapiens
atggggataaacacccgggagctgtttctcaacttcactattgtcttgattacggttattcttatgtggctccttgtgaggtcctatcagtactga

SEQ ID NO: 36 - DNA, 129 bp, Artificial Sequence; identical to SEQ ID NO: 1
  Description of Artificial Sequence Synthetic polynucleotide

SEQ ID NO: 37 - RNA, 157 nt, Artificial Sequence
  Description of Artificial Sequence Synthetic polynucleotide
ucgacuucuuaacccaacagaaggcucgagaagguauauugcuguugacagugagcguaaccgauuucaaauggugcuauagugaagccacagauguauagcaccauuugaaaucgguuaugccuacugccucggacuucaaggggcuagaauucga

SEQ ID NO: 38 - RNA, 254 nt, Artificial Sequence
  Description of Artificial Sequence Synthetic polynucleotide
gaucuuuuaguccuucacuucauggggagccuucagagagaguaaugcagccaccagaaaggaugccguugaccgacacagugacugacaggcugcccuggcgaaccgauuucaaauggugcauaccgucuauucuaaagguagcaccauuugaaaucgguuaggauggcagccaucuuaccuuccaucagaggagccucaccguacccaggaagaaagaaggugaaagaggaaugugaaacagguggcuggga

SEQ ID NO: 39 - RNA, 72 nt, Artificial Sequence
  Description of Artificial Sequence Synthetic oligonucleotide
cuugggaauggcaagguagcaccauuugaaaucgguuacgauuucaaauggugcugucuugcuauacccaga

(19) United States
(12) Patent Application Publication
(10) Pub. No.: US 2022/0031865 A1
(43) Pub. Date: Feb. 3, 2022

(54) COMBINATION THERAPY FOR TREATING MUSCULAR DYSTROPHY

(71) Applicant: Solid Biosciences Inc., Cambridge, MA (US)

(72) Inventors: Senthil Ramu, Cambridge, MA (US); Joel Schneider, Cambridge, MA (US); Kathy Ye Morgan, Somerville, MA (US); Wen Allen Tseng, Somerville, MA (US); Fatih Ozsolak, Quincy, MA (US); Meghan Soustek-Kramer, Boston, MA (US); Eric Reyes, Boston, MA (US); Sarath Mandava, Malden, MA (US)

(21) Appl. No.: 17/312,259

(22) PCT Filed: Dec. 11, 2019
(86) PCT No.: PCT/US2019/065718

§ 371 Date: Jun. 9, 2021

Related U.S. Application Data
(60) Provisional application No. 62/778,646, filed on Dec. 12, 2018.

(51) Int. Cl. A61K 48/00; A61K 38/39; C12N 15/86; C12N 15/113; C12N 15/11; C12N 9/22

(52) U.S. Cl. CPC A61K 48/0058; A61K 48/0091; A61K 38/39; C12N 15/86; C12N 15/113; C12N 15/11; C12N 2800/80; C12N 2750/14143; C12N 2310/141; C12N 2310/14; C12N 2310/122; C12N 2310/531; C12N 2310/20; C12N 9/22

(57) ABSTRACT

The invention described herein provides gene therapy vectors, such as adeno-associated virus (AAV) vectors, that co-express a functional protein (such as a miniaturized human micro-dystrophin gene product) and one or more additional coding sequences for an RNAi sequence (siRNA, shRNA, miRNA), an antisense sequence, a guide sequence for a gene editing enzyme (such as an sgRNA for CRISPR/Cas9, or a crRNA for CRISPR/Casl2a), and/or a micro RNA, and methods of using the vectors to treat subjects suffering from a muscular dystrophy such as DMD/BMD.

### REFERENCE TO RELATED APPLICATION

This application claims priority to and the benefit of the filing date of U.S. Provisional Patent Application No. 62/778,646, filed on Dec. 12, 2018, the entire contents of which is hereby incorporated herein by reference.

### BACKGROUND OF THE INVENTION

Muscular dystrophy (MD) is a group of diseases that cause progressive weakness and loss of muscle mass. In muscular dystrophy, abnormal genes (mutant genes) produce no functional wild-type proteins needed to form healthy muscle.

Muscular dystrophies have serious debilitating impacts on quality of life of affected patients. Duchenne type muscular dystrophy (DMD) is one of the most devastating muscle diseases affecting 1 in 5,000 newborn males. It is the most well-characterized muscular dystrophy, resulting from mutations in genes encoding members of the dystrophin-associated protein complex (DAPC). These MDs result from membrane fragility associated with the loss of sarcolemmal-cytoskeleton tethering by the DAPC.

Specifically, DMD is caused by mutations in the DMD gene, leading to reductions in DMD mRNA and the absence of dystrophin or functional dystrophin, a 427 kDa sarcolemmal protein associated with the dystrophin-associated protein complex (DAPC) (Hoffman et al., Cell 51(6):919-928, 1987). The DAPC is composed of multiple proteins at the muscle sarcolemma that form a structural link between the extra-cellular matrix (ECM) and the cytoskeleton via dystrophin, an actin binding protein, and alpha-dystroglycan, a laminin-binding protein. These structural links act to stabilize the muscle cell membrane during contraction, and protect against contraction-induced damage.

Loss of dystrophin as a result of DMD gene mutations disrupts the dystrophin glycoprotein complex, leading to increased muscle membrane fragility. A cascade of events including influx of calcium into the sarcoplasm, activation of proteases and proinflammatory cytokines, and mitochondrial dysfunction results in progressive muscle degeneration. In addition, displacement of neuronal nitric oxide synthase (nNOS) contributes to tissue ischemia, increased oxidative stress, and reparative failure. Disease progression is characterized by increasing muscle necrosis, fibrosis, and fatty tissue replacement and a greater degree of fiber size variation seen in subsequent muscle biopsies.

Accumulated evidence suggests that abnormal elevation of intracellular Ca²⁺ (Ca²⁺ᵢ) is an important, early pathogenic event that initiates and perpetuates disease progression in DMD. The normal function of sarco/endoplasmic reticulum Ca²⁺ ATPase (SERCA) pump accounts for >70% of Ca²⁺ removal from the cytosol and proper muscle contraction. Reduction in SERCA activity therefore has been considered as a primary cause of Ca²⁺ᵢ overload and muscle dysfunction in DMD.

Currently there is no cure for DMD. The standard of care includes administering corticosteroids (such as prednisone or deflazacort) to stabilize muscle strength and function, prolonging independent ambulation, and delaying scoliosis and cardiomyopathy; bisphosphonates; and denosumab and recombinant parathyroid hormones.

With the advent of gene therapy, research and clinical trials for DMD treatment has focused on gene replacement or other genetic therapies aimed to at least partially restore dystrophin function. These include supplying a functional copy of the dystrophin gene, such as a dystrophin minigene, or repairing a defective dystrophin gene product by exon skipping and nonsense mutation suppression.

However, due to the broad range of effects cause by the dystrophin mutation, there is a need to treat other secondary symptoms associated with the primary dystrophin mutation.

For example, loss of dystrophin leads to the loss of the dystrophin-associated protein complex (DAPC), which in turn leads to the production of nitric oxide (NO) by nNOS, and abnormal N-nitrosylation of HDAC2. Such abnormally N-nitrosylated HDAC2 dissociates from the chromatin, and releases the inhibition of a cascade of specific microRNAs which in turn lead to a slew of downstream events such as fibrosis and increased oxidative stress.

In particular, with respect to fibrosis, with dystrophin loss, membrane fragility results in sarcolemmal tears and an influx of calcium, triggering calcium-activated proteases and segmental fiber necrosis (Straub et al., Curr. Opin. Neurol. 10(2): 168-175, 1997). This uncontrolled cycle of muscle degeneration and regeneration ultimately exhausts the muscle stem cell population (Sacco et al., Cell 143(7): 1059-1071, 2010; Wallace et al., Annu Rev Physiol 71:37-57, 2009), resulting in progressive muscle weakness, endomysial inflammation, and fibrotic scarring.

Without membrane stabilization from dystrophin or a micro-dystrophin, DMD will manifest uncontrolled cycles of tissue injury and repair, and ultimately replace lost muscle fibers with fibrotic scar tissue through connective tissue proliferation.

Muscle biopsies taken at the earliest age of diagnosis of DMD (e.g., between 4-5 years old) reveal prominent connective tissue proliferation. Muscle fibrosis is deleterious in multiple ways. It reduces normal transit of endomysial nutrients through connective tissue barriers, reduces the blood flow and deprives muscle of vascular-derived nutritional constituents, and functionally contributes to early loss of ambulation through limb contractures. Over time, treatment challenges multiply as a result of marked fibrosis in muscle. This can be observed in muscle biopsies comparing connective tissue proliferation at successive time points. The process continues to exacerbate leading to loss of ambulation and accelerating out of control, especially in wheelchair-dependent patients.

Thus fibrotic infiltration is profound in DMD, and is a significant impediment to any potential therapy. In this regard, gene replacement therapy alone is usually hampered by the severity of fibrosis, already present in very young children with DMD.

Fibrosis is characterized by the excessive deposits of ECM matrix proteins, including collagen and elastin. ECM proteins are primarily produced from cytokines such as TGF that is released by activated fibroblasts responding to stress and inflammation. Although the primary pathological feature of DMD is myofiber degeneration and necrosis, fibrosis as a pathological consequence has equal repercussions. The over-production of fibrotic tissue restricts muscle regeneration and contributes to progressive muscle weakness in the DMD patient.

In one study, the presence of fibrosis on initial DMD muscle biopsies was highly correlated with poor motor outcome at a 10-year follow-up (Desguerre et al., J Neuropathol Exp Neurol 68(7):762-767, 2009). These results point to fibrosis as a major contributor to DMD muscle dysfunction and highlight the need to develop therapies that reduce fibrotic tissue.

Most anti-fibrotic therapies that have been tested in mdx mice act to block fibrotic cytokine signaling through inhibition of the TGF pathway.

MicroRNAs (miRNAs) are single-stranded RNAs of ˜22 nucleotides that mediate gene silencing at the post-transcriptional level by pairing with bases within the 3′ UTR of mRNA, inhibiting translation or promoting mRNA degradation. A seed sequence of 7 bp at the 5′ end of the miRNA targets the miRNA; additional recognition is provided by the remainder of the targeted sequence, as well as its secondary structure. MiRNAs play an important role in muscle disease pathology and exhibit expression profiles that are uniquely dependent on the type of muscular dystrophy in question (Eisenberg et al., Proc Natl Acad Sci U.S.A. 104(43):17016-17021, 2007). A growing body of evidence suggests that miRNAs are involved in the fibrotic process in many organs including heart, liver, kidney, and lung (Jiang et al., Proc Natl Acad Sci U.S.A. 104(43):17016-17021, 2007).

Recently, the down-regulation of miR-29 was shown to contribute to cardiac fibrosis (Cacchiarelli et al., Cell Metab 12(4):341-351, 2010). Reduced expression of miR-29 was genetically linked with human DMD patient muscles (Eisenberg et al., Proc Natl Acad Sci U.S.A. 104(43):17016-17021, 2007).

The miR-29 family consists of three family members expressed from two bicistronic miRNA clusters. MiR-29a is coexpressed with miR-29b (miR-29b-1); miR-29c is co-expressed with a second copy of miR-29b (miR-29b-2). The miR-29 family shares a conserved seed sequence, and miR-29a and miR-29b each differ by only one base from miR-29c. Furthermore, electroporation of miR-29 plasmid (a cluster of miR-29a and miR-29b-1) into mdx mouse muscle reduced the expression levels of ECM components, collagen and elastin, and strongly decreased collagen deposition in muscle sections within 25 days post-treatment (Cacchiarelli et al., Cell Metab 12(4):341-351, 2010).

Adeno-associated virus (AAV) is a replication-deficient parvovirus, the single-stranded DNA genome of which is about 4.7 kb in length, including 145 nucleotide inverted terminal repeat (ITRs).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells, allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids, which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther 8:659-669 (1997); Kessler et al., Proc Nat. Acad Sc. U.S.A. 93:14082-14087 (1996); and Xiao et al., J Virol 70: 8098-8108 (1996). See also, Chao et al., Mol Ther 2:619-623 (2000) and Chao et al., Mol Ther 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci U.S.A. 94: 5804-5809 (1997) and Murphy et al., Proc Natl Acad Sci U.S.A. 94: 13921-13926 (1997). Moreover, Lewis et al., J Virol 76: 8769-8775 (2002) demonstrated that skeletal myofibers possess the necessary cellular factors for correct antibody glycosylation, folding, and secretion, indicating that muscle is capable of stable expression of secreted protein therapeutics.

While gene therapy using AAV vectors has fueled significant investments into the sector, significant challenges remain for commercialization. Recombinant viral vector production is seen as complex, with the production scale-up regarded as a major challenge technically, and a large barrier for commercialization.

Specifically, reported clinical doses for AAV-based viral vectors range from 10¹¹ to 10¹⁴ genomic particles (vector genomes; vg) per patient dependent on therapeutic area. Thus, from a wider gene therapy development perspective, current scale-up approaches fall short of supplying the required number of doses to allow later Phase (e.g., Phases II/III) trails to progress, thus retarding the development of gene therapy products. This is supported by the fact that the majority of clinical studies have been very small, performed on <100 patients (and in some cases <10), using adherent cell transfection processes that generate very modest amounts of product. When predicted amounts of virus required for later phase development are compared to current productivities (e.g., 5×10¹¹ vg from single 10 layer cell factory), there is real concern that this approach will fall short of the material requirements for late phase and in-market needs for even ultra-orphan diseases, which have high dose and small patient cohorts, let alone more “standard” gene therapy indications.

As is stated by Clement and Grieger in a recent review article (Molecular Therapy—Methods & Clinical Development (2016) 3, 16002; doi:10.1038/mtm.2016.2): “[t]he use of rAAV in the clinical setting has underscored the urgent need for production and purification systems capable of generating very large amounts of highly pure rAAV particles. Typical FDA-approved investigational new drug includes extensive preclinical studies for toxicology, safety, dose, and bio-distribution assessments, with vector requirements often reaching the 1E15 to 1E16 vector genome range. Manufacturing such amounts, although technically feasible, still represents an incredible effort when using the current production systems.”

This problem is particularly acute for AAV vectors that are desirably delivered systematically (as opposed to locally). In a recent article, Adamson-Small et al. (Molecular Therapy—Methods & Clinical Development (2016) 3, 16031; doi:10.1038/mtm.2016.31) stated that “[c]urrent limitations in vector production and purification have hampered widespread implementation of clinical candidate vectors, particularly when systemic administration is considered . . . . This holds specifically true for the treatment of inherited genetic diseases such as muscular dystrophies, when body-wide gene transfer may be required, relying on systemic dosing often at high AAV doses.” Indeed, previous studies of rAAV in clinical trials for muscular dystrophy have delivered vector via intramuscular injection often due to the lack of large-scale manufacturing capabilities to generate the amounts needed to support systemic administration. Systematic delivery of two AAV vectors in combination therapy poses even a greater challenge in terms of producing sufficient quantities of high quality AAV vectors required for the combination therapy.

Thus, functional improvement in patients suffering from DMD and other muscular dystrophies require both gene restoration and reduction of symptoms associated with a number of secondary cascades such as fibrosis. Alternatively or in addition, muscular dystrophies may benefit from treatments simultaneously targeting different disease-causing pathways. There is a need for methods of reducing such secondary cascade symptoms (e.g., fibrosis) that may be paired with gene restoration methods for more effective treatments of DMD and other muscular dystrophies. Such combination therapy must also overcome the significant clinical and commercialization challenge of producing sufficient quantities of gene therapy vectors to deliver both therapeutic components to the target tissue, particularly in the setting of systematic delivery of gene therapy vectors.

### SUMMARY OF THE INVENTION

The invention described herein provides a viral vector for gene therapy, comprising a polynucleotide sequence that simultaneously encodes a first polypeptide or a first RNA, and a second polypeptide or a second RNA.

For example, the vector may simultaneously encode a first therapeutic protein and a second therapeutic RNA.

However, either the first or the second RNA, or both, may be a non-coding RNA that does not produce a protein or polypeptide. Such non-coding RNA can be microRNA (miR), shRNA (short hairpin RNA), piRNA, snoRNA, snRNA, exRNA, scaRNA, long ncRNAs such as Xist and HOTAIR, anti-sense RNA, or precursor thereof, preferably with therapeutic value, e.g., those associated with diseases such as cancer, autism, Alzheimer's disease, Cartilage-hair hypoplasia, hearing loss, and Prader-Willi syndrome, particularly various types of muscular dystrophies (MDs), including DMD/BMD.

Such non-coding RNA can also be the single or multiple guide RNA(s) of a CRISPR/Cas9 protein, or a CRISPR RNA (crRNA) of a CRISPR/Cas12a (formerly Cpf1) protein.

Thus in one aspect, the invention provides a recombinant viral vector comprising: a) a polynucleotide encoding a functional gene or protein of interest (GOI), such as one effective to treat a muscular dystrophy, wherein said polynucleotide comprises a 3′-UTR coding region, and is immediately 3′ to a heterologous intron sequence that enhances expression of the functional protein encoded by the polynucleotide; b) a control element (e.g., a muscle-specific control element) operably linked to and drives the expression of the polynucleotide; and, c) one or more coding sequences inserted in the intron sequence or in the 3′-UTR coding region; wherein said one or more coding sequences independently encode: an RNAi sequence (siRNA, shRNA, miRNA), an antisense sequence, a guide sequence for a gene editing enzyme (such as a single guide RNA (sgRNA) for CRISPR/Cas9, or acrRNA for CRISPR/Cas12a), a microRNA (miRNA), and/or a miRNA inhibitor.

In certain embodiments, the recombinant viral vector is a recombinant AAV (adeno associated viral) vector or a recombinant lentiviral vector.

In a related aspect, the invention provides a recombinant AAV (rAAV) vector comprising: a) a polynucleotide encoding a functional protein effective to treat a muscular dystrophy, wherein said polynucleotide comprises a 3′-UTR coding region, and is immediately 3′ to a heterologous intron sequence that enhances expression of the functional protein encoded by the polynucleotide; b) a muscle-specific control element operably linked to and drives the expression of the polynucleotide; and, c) one or more coding sequences inserted in the intron sequence or in the 3′-UTR coding region; wherein said one or more coding sequences independently encode: an RNAi sequence (siRNA, shRNA, miRNA), an antisense sequence, a microRNA (miRNA), and/or a miRNA inhibitor.

In a particular embodiment, the invention described herein provides a viral vector, such as a recombinant AAV vector, that comprises: a) a dystrophin microgene or minigene encoding a functional micro-dystrophin protein (e.g., microD5), wherein said dystrophin microgene or minigene comprises a 3′-UTR coding region, and is immediately 3′ to a heterologous intron sequence that enhances expression of the dystrophin microgene or minigene; b) a muscle-specific control element operably linked to and drives the expression of the dystrophin microgene or minigene; and, c) one or more (e.g., 1, 2, 3, 4, or 5) coding sequence(s) inserted in the intron sequence or in the 3′-UTR coding region; wherein said one or more coding sequence(s) independently encode(s): an RNAi sequence (siRNA, shRNA, miRNA), an antisense sequence, a microRNA (miRNA), and/or a miRNA inhibitor.

In certain embodiments, the functional dystrophin protein is microD5, and/or the muscle-specific control element/promoter is CK promoter.

The invention is partly based on the surprising discovery that the one or more coding sequence(s) can be inserted into certain positions, such as heterologous introns, while both the functional protein (such as the dystrophin microgene or minigene product) and one or more coding sequences can be expressed inside the infected target cells (e.g., muscle cells) without significant reduction in expression compared to similar vector constructs encompassing only the functional protein (such as the dystrophin minigene product) or only the one or more coding sequences.

In certain embodiments, the one or more coding sequences are inserted in the 3′-UTR coding region, or after the polyadenylation (polyA) signal sequence (e.g., AATAAA).

In certain embodiments, expression of the functional GOI is substantially unaffected in the presence of the one or more coding sequences (e.g., as compared to otherwise identical control constructs without inserted said one or more coding sequences).

In certain embodiments, in the recombinant AAV (rAAV) vector: a) the polynucleotide is a dystrophin minigene encoding a functional 5-spectrin-like repeat dystrophin protein (e.g., microD5; as described in U.S. Pat. No. 10,479,821, incorporated herein by reference); and/or, b) the muscle-specific control element is a CK promoter operably linked to and drives the expression of the dystrophin minigene.

In certain embodiments, the one or more coding sequences comprise an exon-skipping antisense sequence that induces skipping of an exon of a defective dystrophin, such as skipping any one of exons 45-55 of dystrophin, or exon 44, 45, 51, and/or 53 of dystrophin.

In certain embodiments, the microRNA is miR-1, miR-133a, miR-29c, miR-30c, and/or miR-206. For example, when the microRNA is miR-29c, the miR-29c optionally has a modified flanking backbone sequence that enhances the processing of the guide strand of miR-29c designed for a target sequence. The modified flanking backbone sequence can be from or based on other miR sequences, such as miR-30, -101, -155, or -451.

In certain embodiments, expression of the microRNA in a host cell is up-regulated by at least about 1.5-15 fold (e.g., about 2-10 fold, about 1.4-2.8 fold, about 2-5 fold, about 5-10 fold, about 2, 3, 4, 5, 6, 7, 8, 9, 10, 11, 12, 13, 14, or about 15 fold) compared to endogenous expression of the microRNA in the host cell.

In certain embodiments, the RNAi sequence is an shRNA against sarcolipin (shSLN).

In certain embodiments, the one or more coding sequences encode one or more identical or different shRNAs against sarcolipin (shSLN).

In certain embodiments, the shRNA reduces sarcolipin mRNA and/or sarcolipin protein expression by at least about 50%.

In certain embodiments, the GOI is CRISPR/Cas9, and the guide sequence is the sgRNA; or wherein the GOI is CRISPR/Cas12a, and the guide sequence is the crRNA.

In certain embodiments, the RNAi sequence (siRNA, shRNA, miRNA), the antisense sequence, said CRISPR/Cas9 sgRNA, said CRISPR/Cas12a crRNA and/or the microRNA antagonizes the function of one or more target genes, such as an inflammatory gene, an activator of NF-κB signaling pathway (e.g., TNF-α, IL-1, IL-1β, IL-6, Receptor activator of NF-κB (RANK), and Toll-like receptors (TLRs)), NF-κB, a downstream inflammatory cytokine induced by NF-κB, a histone deacetylase (e.g., HDAC2), TGF-β, connective tissue growth factor (CTGF), collagens, elastin, a structural component of the extracellular matrix, Glucose-6-phosphate dehydrogenase (G6PD), myostatin, phosphodiesterase-5 (PED-5) or ACE, VEGF decoy-receptor type 1 (VEGFR-1 or Flt-1), and hematopoietic prostaglandin D synthase (HPGDS).

In certain embodiments, in the vector, e.g., the recombinant AAV (rAAV) vector: a) the polynucleotide encodes a functional fukutin (FKTN) protein; and/or, b) the one or more coding sequences encode an exon-skipping antisense sequence that restores correct exon 10 splicing in a defective FKTN gene in a Fukuyama congenital muscular dystrophy (FCMD) patient.

In certain embodiments, in the vector, e.g., the recombinant AAV (rAAV) vector: a) the polynucleotide encodes a functional LAMA2 protein; and/or, b) the one or more coding sequences encode an exon-skipping antisense sequence that restores expression of the C-terminal G-domain (exons 45-64), particularly G4 and G5 of a defective LAMA2 gene in a Merosin-deficient congenital muscular dystrophy type 1A (MDC1A) patient.

In certain embodiments, in the vector, e.g., the recombinant AAV (rAAV) vector: a) the polynucleotide encodes a functional DMPK protein, or a CLCN1 gene; and/or, b) the RNAi sequence (siRNA, shRNA, miRNA), the antisense sequence, or the microRNA (miRNA) targets expanded repeats of mutant transcripts in a defective DMPK gene, or encodes an exon-skipping antisense sequence leading to the skipping of exon 7A in CLCN1 gene in a DM1 patient.

In certain embodiments, in the vector, e.g., the recombinant AAV (rAAV) vector: a) the polynucleotide encodes a functional DYSF protein; and/or, b) one or more coding sequences encode an exon-skipping antisense sequence leading to the skipping of exon 32 in a defective DYSF gene in a dysferlinopathy (LGMD2B or MM) patient.

In certain embodiments, in the vector, e.g., the recombinant AAV (rAAV) vector: a) the polynucleotide encodes a functional SGCG protein; and/or, b) one or more coding sequences encode an exon-skipping antisense sequence leading to the skipping of exons 4-7 in a defective LGMD2C gene (e.g., one with the Δ-521T SGCG mutation) in a LGMD2C patient.

In certain embodiments, the heterologous intron coding sequence is SEQ ID NO: 1.

In certain embodiments, the one or more coding sequences are inserted in the intron sequence.

In certain embodiments, expression of the functional protein is not negatively affected by the insertion of said one or more coding sequences.

In certain embodiments, the vector is of the serotype AAV1, AAV2, AAV4, AAV5, AAV6, AAV7, AAVrh74, AAV8, AAV9, AAV10, AAV 11, AAV 12, or AAV 13. In certain embodiments, the vector is a derivative of a known serotype. In certain embodiments, the derivative may exhibit a desired tissue specificity or tropism, a desired immunogenic profile (e.g., not subject to attack by a subject patient's immune system), or other desirable properties for a pharmaceutical composition or gene therapy for various indications.

In certain embodiments, the muscle-specific control element is human skeletal actin gene element, cardiac actin gene element, myocyte-specific enhancer binding factor mef, muscle creatine kinase (MCK), truncated MCK (tMCK), myosin heavy chain (MHC), C5-12, murine creatine kinase enhancer element, skeletal fast-twitch troponin c gene element, slow-twitch cardiac troponin c gene element, slow-twitch troponin i gene element, hypoxia-inducible nuclear factors, steroid-inducible element, or glucocorticoid response element (gre).

In certain embodiments, the muscle-specific control element comprises the nucleotide sequence of SEQ ID NO: 10 or SEQ ID NO: 11 of WO2017/181015 (incorporated herein by reference).

Another aspect of the invention provides a composition comprising any of the vector, e.g., the recombinant viral (AAV) vector of the invention.

In certain embodiments, the composition is a pharmaceutical composition further comprising a therapeutically compatible carrier, diluent, or excipient.

In certain embodiments, the therapeutically acceptable carrier, diluent, or excipient is a sterile aqueous solution comprising 10 mM L-histidine at pH 6.0, 150 mM sodium chloride, and 1 mM magnesium chloride.

In certain embodiments, the composition is in a dosage form of about 10 mL of aqueous solution having at least 1.6×10¹³ vector genomes.

In certain embodiments, the composition has a potency of at least 2×10¹² vector genomes per milliliter.

Another aspect of the invention provides a method of producing the subject composition, comprising producing the vector, e.g., the recombinant AAV vector in a cell and lysing the cell to obtain the vector.

In certain embodiments, the vector is an AAV1, AAV2, AAV4, AAV5, AAV6, AAV7, AAVrh74, AAV8, AAV9, AAV10, AAV 11, AAV 12, or AAV 13 vector.

Another aspect of the invention provides a method of treating a muscular dystrophy or dystrophinopathy in a subject in need thereof, the method comprising administering to the subject a therapeutically effective amount of any one of the recombinant vector, e.g., the recombinant AAV vector of the invention, or any one of the composition of the invention.

In certain embodiments, the recombinant vector, e.g., the recombinant AAV vector or the composition is administered by intramuscular injection, intravenous injection, parental administration or systemic administration.

In certain embodiments, the muscular dystrophy is Duchenne muscular dystrophy or Becker muscular dystrophy.

In certain embodiments, the muscular dystrophy is Duchenne muscular dystrophy, Becker muscular dystrophy, Fukuyama congenital muscular dystrophy (FCMD), dysferlinopathy, myotonic dystrophy, and merosin-deficient congenital muscular dystrophy type 1A, facioscapulohumeral muscular dystrophy (FSHD), congenital muscular dystrophy (CMD), or limb-girdle muscular dystrophy (LGMDR5 or LGMD2C).

Another aspect of the invention provides a kit for preventing or treating DMD or related/associated diseases in a subject, the kit comprising: one or more vector, e.g., the recombinant AAV as described herein, or a composition as described herein; instructions for use (written, printed, electronic/optical storage media, or online); and/or packaging. In certain embodiments, a kit also includes a known MD (e.g., DMD) therapeutic for combination therapy.

It should be understood that any one embodiment described herein, including one described only in the example or claims, can be combined with any one or more other embodiments of the invention unless such combination is expressly disclaimed or improper.

### BRIEF DESCRIPTION OF THE DRAWINGS

FIG. 1 shows schematic drawings (not to scale) showing a representative recombinant viral (e.g., lentiviral or AAV) vector encompassing a Gene of interest (GOI), such as a microdystrophin, minidystrophin or dystrophin minigene as described below (e.g., the 5-spectrin-like-repeat microD5 dystrophin protein described below) and one or more (i.e., five, as shown) additional coding sequences for non-protein coding RNA (ncRNA) such as shRNA, between the two ITR sequences. The additional ncRNA (e.g., shRNA) coding sequences can be the same or different, and appear, in this drawing, to be within the heterologous intron sequence 5′ to the gene of interest (GOI) coding region (e.g., the microdystrophin coding sequence), although the location of the additional coding sequences is not so limited. That is, the coding sequences can be located elsewhere in the AAV vector, such as within the 3′-UTR region, or in both the heterologous intron and the 3′-UTR region. Upon transcription of the AAV vector genome, a pre-processed mRNA encompassing the GOI or dystrophin minigene (e.g., microD5) mRNA and the additionally coded sequences as a fusion RNA is produced. Upon further processing, the GOI, such as dystrophin minigene mRNA (as an anti-DMD drug) and the additional coded sequences, such as the shown shRNA's, are separated.

FIG. 2A shows one specific embodiment of the recombinant viral (e.g., lentiviral or AAV) vector in which a single additional microRNA29c coding sequence is inserted into the 3′-UTR region. Transcription and further processing lead to the creation of the microD5 (labeled as “SGT-001”) mRNA for microdystrophin, and a functional miR29c microRNA. Note that the TAG stop codon, the AATAAA polyA signal sequence, and the miR-29c insertion sequence (which happens to be CA) in this illustrative, non-limiting example are all underlined. Although in this illustration, the miR29c coding sequence was depicted to be inserted after the polyA signal sequence, the same can also be inserted elsewhere, such as in the 3′-UTR region of the mature mRNA that is before the polyA signal sequence. Also see FIG. 12.

FIG. 2B shows another specific embodiment of the recombinant viral (e.g., lentiviral or AAV) vector in which a single additional sarcolipin (SLN) shRNA coding sequence (shSLN) is inserted into the heterologous intron. Transcription and further processing lead to the creation of the microD5 (labeled as SGT-001) mRNA for microdystrophin, and a functional sarcolipin shRNA. Again, the location of insertion for the shSLN is for illustration purpose only, and it can be inserted elsewhere according to the present disclosure, such as in the 3′-UTR region, or either before or after the polyA signal sequence.

FIG. 3 shows DAPI staining for nucleus, and immunofluorescent staining for dystrophin, in cells infected by an AAV vector encoding only the microD5 (labeled as SGT-001) microdystrophin (left), an AAV vector further encoding a microRNA 29c in the heterologous intron (middle), and an AAV vector further encoding a sarcolipin shRNA in the heterologous intron (right). Percentage values represent transfection efficiency, or percentage of cells successfully transfected.

FIG. 4 is a schematic drawing showing an AAV vector encoding a sarcolipin-luciferase reporter fusion. The target location of the shRNA against sarcolipin is also shown.

FIG. 5 shows that the expression of the sarcolipin-luciferase fusion reporter in C2C12 cells is reduced by 86.8%, when the cells were co-transfected by an AAV vector expressing both microD5 and shSLN (“SGT001+SLN”), compared to cells co-transfected by an AAV vector expressing microD5 only (“SGT001”).

FIG. 6A shows that endogenous sarcolipin expression in C2C12 cells (6 days post transfection) was reduced by 55% in C2C12 cells transfected by an AAV vector encoding microD5 and shSLN (labeled as “SGT001-shSLN”), compared to C2C12 cells transfected by an AAV vector encoding only microD5 (labeled as “SGT-001”).

FIG. 6B shows immunofluorescence staining images for endogenous SLN expression, based on which the data in FIG. 6A was compiled.

FIG. 7 shows that expression of shSLN (by transfecting an AAV vector encoding both the microD5 dystrophin and shSLN) in C2C12 cells reduced function of endogenous SLN, such that Calcium reuptake into the sarcoplasmic reticulum is affected over the time course. Controls include cells transfected by an AAV vector encoding only the microD5 dystrophin without shSLN, and non-transfected cells. Relative fluorescent intensity on the Y-axis is based on the measurement of fluorescent intensity of Calcium probe Fluo-8.

FIG. 8A shows that microdystrophin expression in C2C12 cells transfected by SGT-001-shSLN (an AAV vector encoding both the microD5 dystrophin and shSLN) lagged that of C2C12 cells transfected by SGT-001 (an AAV vector encoding the microD5 dystrophin) only, one day post transfection (1d), i.e., at about 20% level, but the microD5 dystrophin minigene expression quickly caught up by day 6 post transfection (6d) (within the margin of error).

FIG. 8B shows immunofluorescence staining images for exogenous microD5 dystrophin minigene expression at 1 day post transfection, based on which the data in FIG. 8A was compiled.

FIG. 8C shows immunofluorescence staining images for exogenous microD5 dystrophin minigene expression at 6 days post transfection, based on which the data in FIG. 8A was compiled.

FIG. 9 shows several exemplary shRNA designs for mouse SLN.

FIG. 10 shows nucleotide sequence comparison between mouse (Subject) and human (Query) sarcolipin sequences, and possible shRNA designs for mice- or human-specific shRNA, as well as shRNA common for mice and human.

FIG. 11 shows representative locations in the AAV vector encoding a dystrophin minigene (microD5, labeled as “SGT-001”) can serve as insertion points for the one or more coding sequences such as the miR-29c coding sequence (as shown) or the coding sequence for the shRNA against SLN. Specifically, multiple locations within an intron of the SGT-001 minigene can be used, though some locations (such as Imir2) may be more preferred due to the lack of negative impact on dystrophin minigene expression.

FIG. 12 is a schematic drawing (not to scale) showing one representative and non-limiting embodiment of the subject recombinant viral (e.g., lentiviral or AAV) vector. In this specific embodiment shown, the control element is the muscle-specific promoter CK8 and the GOI is a version of a functional DMD gene (micro dystrophin or μDys). The coding sequence for the RNAi, miRNA, etc. can be inserted anywhere in the vector where “Transcript” is indicated, e.g., in the intron before the GOI, in the 3′-UTR region, or after the polyA signal sequence. The resulting transcription from the promoter will result in an initial fusion transcript.

FIG. 13 shows the relative miR-29c expression level changes (in folds over the control vector expressing μDys only) in human iPS-derived cardiomyocytes, for the various recombinant viral (e.g., AAV) vectors encoding miR-29c, either as the sole coding sequence in the viral vector (the “Solo” constructs), or as part of the fusion constructs of the present disclosure (the “Fusion” constructs).

FIG. 14 shows relative expression levels of miR-29c in differentiated C2C12 cells or mouse cardiomyocytes for the various recombinant AAV vectors encoding miR-29c, either as the sole coding sequence in the viral vector (the “Solo” constructs), or as part of the fusion constructs of the present disclosure (the “Fusion” constructs).

FIG. 15 shows about 50% knock-down of mouse SLN protein expression levels (bottom row) via a shSLN-μDys fusion construct of the present disclosure, as well as several solo constructs expressing the same shSLN coding sequence. The top row is loading control.

FIG. 16 shows relative expression levels of siSLN (processed siRNA product from the transcribed shSLN) in differentiated C2C12 myotubes or mouse cardiomyocytes for the various recombinant AAV vectors encoding shSLN, either as the sole coding sequence in the viral vector (“Solo”), or as part of the fusion construct of the present disclosure (“Fusion”).

FIG. 17 shows up to ˜90% human SLN mRNA knock-down in human iPS-derived cardiomyocytes by several subject fusion constructs encoding shSLN.

FIG. 18 shows normalized μDys mRNA levels of several Hum-shSLN-μDys fusion constructs in human iPS-derived cardiomyocytes.

FIG. 19 is an image of denaturing agarose gel, suggesting largely intact AAV genomes in the solo and fusion constructs with miR-29c coding sequence.

FIGS. 20A-20C show about 1.4-2.8-fold miR-29c expression up-regulation in left gastrocnemius (FIG. 20A), diaphragm (FIG. 20B), and left ventricle (FIG. 20C), respectively, using a miR-29c-μDys fusion construct of the invention in AAV9 vector.

FIG. 21 shows no reduction of μDys expression at RNA and protein level in gastrocnemius with miR-29c up-regulating fusion AAV9 vector.

FIG. 22 shows up to 50% mSLN mRNA down-regulation in diaphragm, left gastrocnemius (left gast), and atrium, respectively, via AAV9-mediated expression of shSLN-μDys fusion construct relative to μDys-only AAV9. Up to 50% mSLN mRNA down-regulation was also observed in tongue (data not shown).

FIG. 23 shows similar levels of μDys RNA/protein expression in diaphragm via an shSLN-μDys fusion construct of AAV9. Similar results were also obtained for tongue and atrium (data not shown).

FIG. 24 shows that miR-29c solo and miR-29c-μDys fusion constructs of AAV9 reduce serum CK levels.

FIG. 25 shows that miR-29c solo and miR-29c-μDys fusion constructs of AAV9 reduce serum TIMP1 levels.

FIG. 26 shows largely similar biodistribution of miR-29c or shSLN vectors in gastrocnemius from several miR-29c-μDys fusion vectors of AAV9 or shSLN-μDys fusion vectors of AAV9.

FIG. 27 shows similar titer of AAV9 vectors in liver for miR-29c-μDys fusion and shSLN-μDys fusion vs. μDys solo construct.

FIG. 28 shows added benefit of the fusion constructs of the invention over μDys construct alone in diaphragm, based on their effects on two fibrotic marker genes.

FIG. 30 shows predicted 2D structure for a representative modified miR-29c construct based on the miR-30E backbone sequence.

FIG. 31 shows predicted 2D structure for a representative modified miR-29c construct based on the miR-101 backbone sequence.

FIG. 32 shows predicted 2D structure for a representative modified miR-29c construct based on the miR-451 backbone sequence.

### DETAILED DESCRIPTION OF THE INVENTION

Without a parallel approach to treat a varieties of secondary cascade symptoms such as fibrosis and abnormal elevation of intracellular Ca²⁺, it is unlikely that the benefits of exon skipping, stop-codon read-through, or gene replacement therapies can ever be fully achieved. Even small molecules or protein replacement strategies are likely to fail without an approach to reduce symptoms of such secondary cascade events including muscle fibrosis. For example, previous work in aged mdx mice with existing fibrosis treated with AAV micro-dystrophin demonstrated that one could not achieve full functional restoration (Human molecular genetics 22:4929-4937, 2013). It is also known that progression of DMD cardiomyopathy is accompanied by scarring and fibrosis in the ventricular wall.

The present invention is partly directed to gene therapy methods to treat a patient that not only compensate defects in dystrophin and its function by providing a replacement, functional dystrophin minigene, but also directly target one or more secondary cascade genes using one or more additional coding sequences in the same gene therapy vector, thus achieving combination therapy in one compact vector for systematic delivery.

Indeed, the present invention, particularly the recombinant AAV (rAAV) vector of the invention, is not limited to treating DMD. The invention is applicable for treating other muscular dystrophies in which a gene is defective. For example, the recombinant AAV (rAAV) vector of the invention can provide a functional protein and/or one or more coding sequences (such as non-coding RNAs, e.g., RNAi sequence, antisense RNA, miRNA) to treat the muscular dystrophy, wherein the functional protein either provides a wild-type substitute for the defective gene product in the muscular dystrophy, or provides a non-wild-type substitute that is nevertheless effective to treat the muscular dystrophy (e.g., the 5-spectrin-like microD5 dystrophin minigene product).

Thus in one aspect, the invention provides a recombinant viral vector, e.g., a recombinant lentiviral or AAV (rAAV) vector comprising: a) a polynucleotide encoding a functional protein effective to treat the muscular dystrophy in a patient/subject/individual in need of treatment, wherein said polynucleotide comprises a 3′-UTR coding region, and is immediately 3′ to a heterologous intron sequence that enhances expression of the functional protein encoded by the polynucleotide, wherein the corresponding wild-type of the functional protein is defective in a muscular dystrophy, or wherein the functional protein, though not wild-type, is nevertheless effective to treat the muscular dystrophy; b) a control element (e.g., a muscle-specific control element) operably linked to and drives the expression of the polynucleotide; and, c) one or more coding sequences inserted in the intron sequence or in the 3′-UTR coding region or elsewhere in the expression cassette for the functional protein; wherein said one or more coding sequences independently encode: an RNAi sequence (siRNA, shRNA, miRNA), an antisense sequence, a microRNA (miRNA), and/or a miRNA inhibitor.

In a related aspect, the invention described herein can also be used as a viral vector for simultaneously delivering/expressing two or more components of an enzyme-based gene editing system, e.g., such as a target sequence-specific (engineered) nuclease that can create DNA double stranded break (DSB) at a target genomic site/target genomic sequence, and a donor or template sequence that matches the (wild-type or desired) target genome sequence. Such a system makes it possible to utilize the endogenous homologous recombination (HR) processes within the target cell to edit out a defective/undesired target genomic sequence, and replace it with a wild-type or otherwise desired sequence at the desired target genomic location.

For example, the target sequence-specific (engineered) nuclease may include meganucleases (such as those in the LAGLIDADG family) and variants thereof that recognize unique target genomic sequences; Zinc Finger Nucleases (ZFNs); Transcription Activator-Like Effector Nucleases (TALENs); and CRISPR/Cas gene editing enzymes.

In the case of CRISPR/Cas, for example, the subject vector can simultaneously deliver, other than or in addition to the donor sequence, one or more gene editing guide sequence(s) having a desired sequence(s) for targeting one or more target sequence(s), and a compatible editing enzyme that can be encoded by the viral vector as the GOI. Such a viral delivery system can be used to substitute the undesired sequence occurring in the cell, tissue, or organism for the desired sequence. One example of the CRISPR/Cas enzyme system is CRISPR/Cas9 or CRISPR/Cas12a (formerly Cpf1), and one or more required guide sequences (e.g., single guide RNA or sgRNA for Cas9, or crRNA for Cas12a) to a target cell. Cas9 includes the wild-type Cas9 and functional variants thereof. Several Cas9 variants are about the same size as the micro Dystrophin gene, and can be the functional GOI encoded by the viral vector of the invention. Cas12a is even smaller than Cas9 and can also be encoded as the GOI. In certain embodiments, the Cas genes encoded by the viral constructs may or may not have UTR and/or intron elements.

In a related aspect, the invention provides a recombinant lentiviral vector for use in ex vivo or in vivo gene therapy. In ex vivo gene therapy, cultured host cells are transfected in vitro using a subject viral vector to express the gene of interest, and then transplanted into the body. In vivo gene therapy is a direct method of inserting the genetic material into the targeted tissue, and transduction takes place within the patient's own cells. Thus the lentiviral vector of the invention may comprise: a) a polynucleotide encoding a functional protein effective to treat the muscular dystrophy in a patient/subject/individual in need of treatment, wherein said polynucleotide comprises a 3′-UTR coding region, and is immediately 3′ to a heterologous intron sequence that enhances expression of the functional protein encoded by the polynucleotide, wherein the corresponding wild-type of the functional protein is defective in a muscular dystrophy, or wherein the functional protein, though not wild-type, is nevertheless effective to treat the muscular dystrophy; b) a control element (e.g., a muscle-specific control element) operably linked to and drives the expression of the polynucleotide; and, c) one or more coding sequences inserted in the intron sequence or in the 3′-UTR coding region or elsewhere in the expression cassette; wherein said one or more coding sequences independently encode: an RNAi sequence (siRNA, shRNA, miRNA), an antisense sequence, a microRNA (miRNA), and/or a miRNA inhibitor.

As used herein, and depending on context, the term “fusion” may have different meanings, including fusion proteins, fusion RNA transcripts in which more than one encoded sequence may be present (such as the coding sequence for the GOI and the coding sequence for one or more RNAi agents etc inserted into/embedded in the 3-UTR region or intron sequences of the GOI, and fusion constructs in which the viral vectors contain coding sequences for the GOI and the one or more RNAi agents, etc.

In certain embodiments, the one or more coding sequences are inserted in the 3′-UTR coding region, or after the polyadenylation (polyA) signal sequence (e.g., AATAAA).

In certain embodiments, expression of the functional GOI is up- or down-regulated due to the presence of the one or more coding sequences (e.g., as compared to otherwise identical control constructs without inserted said one or more coding sequences).

In certain embodiments, expression of the functional GOI is substantially unaffected in the presence of the one or more coding sequences (e.g., as compared to otherwise identical control constructs without inserted said one or more coding sequences).

As used herein, “muscular dystrophy (MD)” includes a group of diseases characterized by progressive weakness and loss of muscle mass, due to abnormal genes or gene mutations that interfere with the production of wild-type proteins needed to form healthy muscle. MD includes Duchenne muscular dystrophy (DMD); Becker muscular dystrophy (BMD); a congenital muscular dystrophy (CMD), particularly one with an identified genetic mutation, such as the ones described hereinbelow, including Fukuyama congenital muscular dystrophy (FCMD) and Merosin-deficient congenital muscular dystrophy type 1A (MDC1A); dysferlinopathy (LGMD2B and Miyoshi myopathy); myotonic dystrophy; limb-girdle muscular dystrophy (LGMD) such as LGMD2C; and Facioscapulohumeral (FSHD).

As used herein, “patient,” “subject,” and “individual” are used interchangeably to include a mammalian (e.g., human) subject to be treated, diagnosed, and/or to obtain a biological sample from in the subject methods. Typically, the subject is affected or likely to be affected with DMD and the other related diseases described herein, and in some embodiments, DMD and associated cardiomyopathy and dystrophic cardiomyopathy. In a particular embodiment, a subject is a human child or adolescent (e.g., no more than 18 years old, 15 years old, 12 years old, 10 years old, 8 years old, 5 years old, 3 years old, 1 year old, 6 months old, 3 months old, 1 month old, etc.). In a particular embodiment, the child or adolescent is male. In another particular embodiment, a subject is a human adult (e.g., >18 years old), such as a male adult.

The full-length dystrophin gene is 2.6 mb and encodes 79 exons. The 11.5-kb coding sequence translates into a 427-kD protein. Dystrophin can be divided into four major domains, including the N-terminal domain, rod domain, cysteine-rich domain, and C-terminal domain. The rod domain can be further divided into 24 spectrin-like repeats and four hinges.

A functional “dystrophin minigene” or “dystrophin microgene” has less than 24 spectrin-like repeats and one or more hinge region/s compatible with gene therapy delivery vectors (adenoviral and lentiviral) and have been described in U.S. Pat. Nos. 7,001,761, 6,869,777, 8,501,920, 7,892,824, U.S. Ser. No. 10/479,821, and U.S. Ser. No. 10/166,272 (all incorporated herein by reference).

In one embodiment, the muscular dystrophy is DMD or BMD, and in the recombinant AAV (rAAV) vector: a) the polynucleotide is a dystrophin minigene encoding a functional 5-spectrin-like repeat dystrophin protein (such as the microD5 dystrophin protein as described in U.S. Pat. No. 10,479,821, incorporated herein by reference); and/or, b) the muscle-specific control element is a CK promoter operably linked to and drives the expression of the dystrophin minigene.

As used herein, “microD5,” “microdystrophin minigene encoded by SGT-001,” or “SGT-001” for short, refers to a specific engineered 5-repeat microdystrophin protein that contains, from N- to C-terminus, the N-terminal actin binding domain, Hinge region 1 (H1), spectrin-like repeats R1, R16, R17, R23, and R24, Hinge region 4 (H4), and the C-terminal dystroglycan binding domain of the human full-length dystrophin protein. The protein sequence of this 5-repeat microdystrophin and the related dystrophin minigene are described in U.S. Pat. No. 10,479,821 & WO2016/115543 (incorporated herein by reference).

In certain embodiments, the dystrophin minigene encoding a functional dystrophin protein different from microD5 with respect to, for example, the specific spectrin-like repeats, and/or the number of spectrin-like repeats (e.g., comprising a minimum of 4, 5, or 6 spectrin-like repeats of the human dystrophin, preferably including 1, 2, or 3 most N- and/or most C-terminal repeats). One or more spectrin-like repeats of the human dystrophin may also be substituted by spectrin-like repeats from utrophin or spectrin. In certain embodiments, the dystrophin minigene is smaller than the 5 kb packaging limit of AAV viral vectors, preferably no more than 4.9 kb, 4.8 kb, 4.6 kb, 4.5 kb, 4.4 kb, 4.3 kb, 4.2 kb, 4.1 kb, or 4 kb.

In certain embodiments, the dystrophin minigene encodes a micro-dystrophin protein that is, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to microD5, wherein the protein retains micro-dystrophin activity.

In certain embodiments, the micro-dystrophin is encoded by a nucleotide sequence that has at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to a polynucleotide sequence encoding the microD micro-dystrophin. The polynucleotide is optionally codon optimized for expression in a mammal, such as in a human.

In certain embodiments, the nucleotide sequence hybridizes under stringent conditions to the nucleic acid sequence encoding the microD5 micro-dystrophin, or compliments thereof, and encodes a functional micro-dystrophin protein.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015 M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989).

More stringent conditions (such as higher temperature, lower ionic strength, higher formamide, or other denaturing agent) may also be used, however, the rate of hybridization will be affected. In instances wherein hybridization of deoxyoligonucleotides is concerned, additional exemplary stringent hybridization conditions include washing in 6×SSC 0.05% sodium pyrophosphate at 37° C. (for 14-base oligoes), 48° C. (for 17-base oligos), 55° C. (for 20-base oligos), and 60° C. (for 23-base oligos).

Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodS04, (SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: A Practical Approach, Ch. 4, IRL Press Limited (Oxford, England). Hybridization conditions can be adjusted by one skilled in the art in order to accommodate these variables and allow DNAs of different sequence relatedness to form hybrids.

Additional dystrophin minigene sequences can be found in, for example, US2017/0368198 (incorporated herein by reference), and SEQ ID NO: 7 of WO 2017/181015 (incorporated herein by reference).

In certain embodiments, the nucleotide sequence encoding any dystrophin minigene such as microD5 can be any one based on the disclosed protein sequence. Preferably, the nucleotide sequence is codon optimized for expression in human.

The micro-dystrophin protein provides stability to the muscle membrane during muscle contraction, e.g., micro-dystrophin acts as a shock absorber during muscle contraction.

In certain embodiments, at least one of the one or more coding sequences target one of the secondary cascade genes in DMD.

For example, in certain embodiments, at least one of the one or more coding sequences encodes a microRNA, such as miR-1, miR-133a, miR-29 particularly miR29c, miR-30c, and/or miR-206. For example, miR-29c directly reduce the three primary components of connective tissue (e.g., collagen 1, collagen 3 and fibronectin) to reduce fibrosis.

“Fibrosis” as used herein refers to the excessive or unregulated deposition of extracellular matrix (ECM) components and abnormal repair processes in tissues upon injury including skeletal muscle, cardiac muscle, liver, lung, kidney, and pancreas. The ECM components that are deposited include fibronectin and collagen, e.g., collagen 1, collagen 2 or collagen 3.

As used herein, “miR-29” refers to one of miR-29a, -29b, or -29c. In certain embodiments, miR-29 refers to miR-29c.

While not wishing to be bound by any particular theory, it is believed that the expressed miR29 (such as miR-29a, miR-29b, or miR-29c) binds to the 3′ UTR of the collagen and fibronectin genes to down-regulate expression of these target genes.

In another embodiment, at least one of the one or more coding sequences encodes an RNAi sequence, such as an shRNA against sarcolipin (shSLN). The one or more coding sequences may encode identical or different shRNAs against sarcolipin (shSLN). In certain embodiments, the shRNA reduces sarcolipin mRNA and/or sarcolipin protein expression by at least about 50%.

As used herein, “sarcolipin (SLN),” “sarcolipin protein,” “SLN protein,” “sarcolipin polypeptide” and “SLN polypeptide” are used interchangeably to include an expression product of a SLN gene, such as the native human SLN protein having the amino acid sequence of (MGINTRELFLNFTIVLITVILMWLLVRSYGY) (SEQ ID NO: 1), accession number NP_003054.1. The term preferably refers to the human SLN. The term may also be used to refer to a variant SLN protein that differs from SEQ ID NO: 1 by 1 amino acid, 2 amino acids, 3 amino acids, 4 amino acids, 5 amino acids, 6 amino acids, 7 amino acids, or 8 amino acids, optionally the differences are within residues 2-5, 10, 14, 17, 20, and 30, preferably 2-5 and 30. The term may also be used to refer to a variant SLN protein that are identical to SEQ ID NO: 1 at residues 6-29, or differ in residues 6-29 by up to 1, 2, or 3 conservative substitutions such as L→I and/or I→V. Optionally, the variant SLN has a G30Q substitution. The variants displays a functional activity of a native SLN protein, which may include: phosphorylation, dephosphorylation, nitrosylation and/or ubiquitination of SLN, or binding to a SERCA and/or reduce the rate of calcium import by SERCA into the sarcoendoplasmic reticulum through, for example, uncoupling of Ca²⁺ transport from ATP hydrolysis, or its role in energy metabolism and regulation of weight gain.

As used herein, “SLN gene,” “SLN polynucleotide,” and “SLN nucleic acid” are used interchangeably to include a native human SLN-encoding nucleic acid sequence, e.g., the native human SLN gene (RefSeq Accession: NM_003063.2), a nucleic acid having sequences from which a SLN cDNA can be transcribed; and/or allelic variants and homologs of the foregoing, such as a polynucleotide encoding any of the variant SLN described herein. The terms encompass double-stranded DNA, single-stranded DNA, and RNA.

In another embodiment, the one or more additional coding sequences of the subject vector may be targeting any other genes associated with one of the secondary cascade events resulting from the loss of dystrophin gene, such as inflammatory gene, an activator of NF-κB signaling pathway (e.g., TNF-α, IL-1, IL-1β, IL-6, Receptor activator of NF-κB (RANK), and Toll-like receptors (TLRs)), NF-κB, a downstream inflammatory cytokine induced by NF-κB, a histone deacetylase (e.g., HDAC2), TGF-β, connective tissue growth factor (CTGF), ollagens, elastin, a structural component of the extracellular matrix, Glucose-6-phosphate dehydrogenase (G6PD), myostatin, phosphodiesterase-5 (PED-5) or ACE, VEGF decoy-receptor type 1 (VEGFR-1 or Flt-1), and hematopoietic prostaglandin D synthase (HPGDS). The one or more additional coding sequences can be an RNAi sequence (siRNA, shRNA, miRNA), an antisense sequence, and/or a microRNA that antagonizes the function of the above target genes.

The design of the subject recombinant vectors can simultaneously target one or more (e.g., 1, 2, 3, 4, 5) such secondary cascade genes or pathways, such as SLN, microRNA, etc.

For example, in certain embodiments, one of the additional coding sequence of the subject vector may be an RNAi sequence (siRNA, shRNA, miRNA) or an antisense sequence designed to down-regulate SLN expression, hence at least partially alleviate the secondary defect of abnormal elevation of intracellular Ca²⁺ in dystrophy muscle by increasing the reuptake of calcium by SERCA.

In certain alternative embodiments, instead of or in addition to targeting one of the secondary cascade genes, at least one of the one or more coding sequences may be an exon-skipping antisense sequence that induces skipping of an exon of a defective endogenous dystrophin, such as any one of exons 45-55 of dystrophin, or exon 44, 45, 51, and/or 53 of dystrophin, thus further enhancing the therapeutic effect of the dystrophin minigene (e.g., microD5).

As used herein, an “exon skipping” or “splice-switching” antisense oligonucleotide (AON) is a type of antisense sequence that is RNase-H-resistant, and acts to modulate pre-mRNA splicing and correct splicing defects in the pre-mRNA. In antisense-mediated exon skipping therapy, AONs are usually used to block specific splicing signals and induce specific skipping of certain exons. This leads to the correction of the reading frame of a mutated transcript, such that it can be translated into an internally deleted but partially functional protein.

In a specific aspect, the invention provides a recombinant AAV (rAAV) vector encoding both a dystrophin minigene coding sequence (such as microD5/SGT-001), and one or more additional sequences for targeting one or more additional target genes involved in a secondary cascade resulting from loss of dystrophin function. Such construct comprises both a dystrophin minigene, and one or more additional coding sequences inserted into a heterologous intron 5′ to the dystrophin minigene, and/or into the 3′-UTR region of the dystrophin minigene.

Specifically, in one aspect, the invention provides a recombinant AAV (rAAV) vector comprising: a) a dystrophin minigene encoding a functional micro-dystrophin protein, wherein said dystrophin minigene comprises a 3′-UTR coding region, and is immediately 3′ to a heterologous intron sequence that enhances expression of the dystrophin minigene; b) a muscle-specific control element operably linked to and drives the expression of the dystrophin minigene; and, c) one or more (e.g., 1, 2, 3, 4, or 5) coding sequence(s) inserted in the intron sequence or in the 3′-UTR coding region; wherein said one or more coding sequence(s) independently encode(s): an RNAi sequence (siRNA, shRNA, miRNA), an antisense sequence, a microRNA (miRNA), and/or a miRNA inhibitor.

For example, the rAAV vector may comprise a polynucleotide sequence expressing miR-29 (e.g., miR-29c), such as a nucleotide sequence comprising the miR-29c target guide strand (ACCGATTTCAAATGGTGCTAGA, SEQ ID NO: 3 of WO2017/181015 or, incorporated herein by reference), the miR-29c guide strand (TCTAGCACCATTTGAAATCGGTTA, SEQ ID NO: 4 of WO2017/181015, incorporated herein by reference) and the natural miR-30 back bone and stem loop (GTGAAGCCACAGATG, SEQ ID NO: 5 of WO2017/181015, incorporated herein by reference).

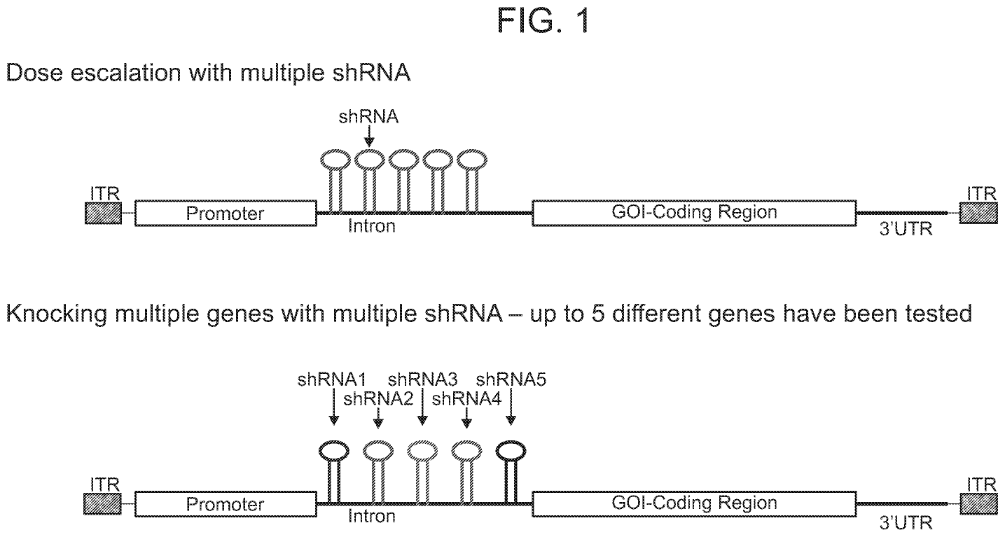

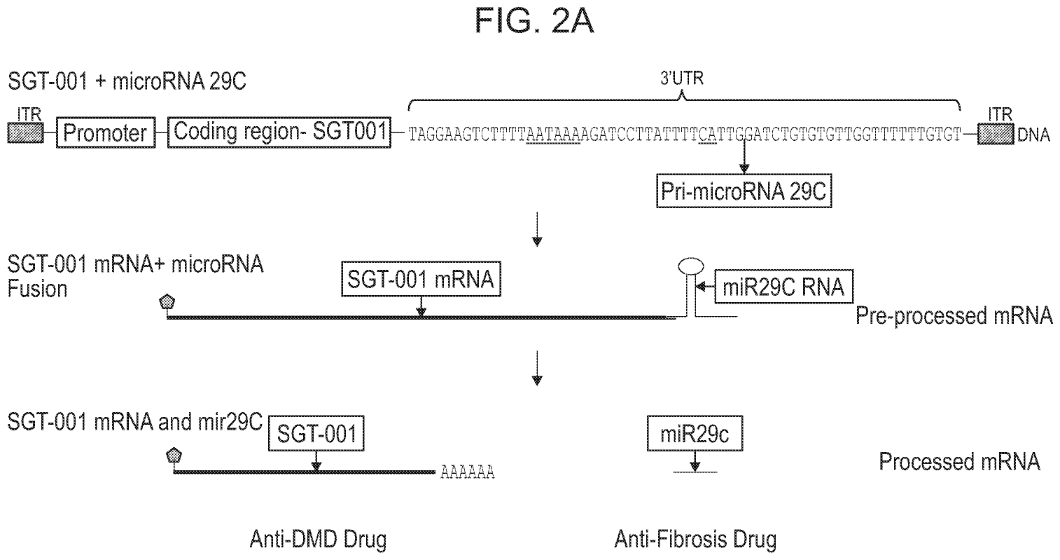

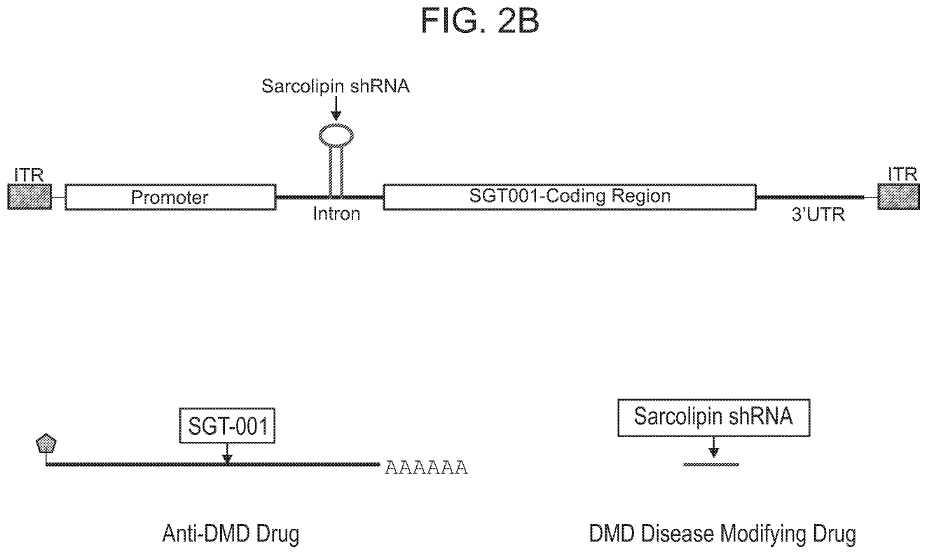

An exemplary polynucleotide sequence comprising the miR-29c cDNA in a miR-30 backbone is set out as SEQ ID NO: 2 and FIG. 1 of WO2017/181015 (incorporated herein by reference).

In certain embodiments, the microRNA-29 coding sequence encodes miR-29c.

In certain embodiments, miR-29c optionally has a modified flanking backbone sequence that enhances the processing of the guide strand of miR-29c designed for a target sequence. For example, the modified flanking backbone sequence can be from or based on that of miR-30 (miR-30E), -101, -155, or -451.

In certain embodiments, the microRNA is miR-1, miR-133a, miR-30c, and/or miR-206.

In certain embodiments, expression of said microRNA in a host cell is up-regulated by at least about 1.5-15 fold (e.g., about 2-10 fold, about 1.4-2.8 fold, about 2-5 fold, about 5-10 fold, about 2, 3, 4, 5, 6, 7, 8, 9, 10, 11, 12, 13, 14, or about 15 fold) compared to endogenous expression of said microRNA in said host cell.

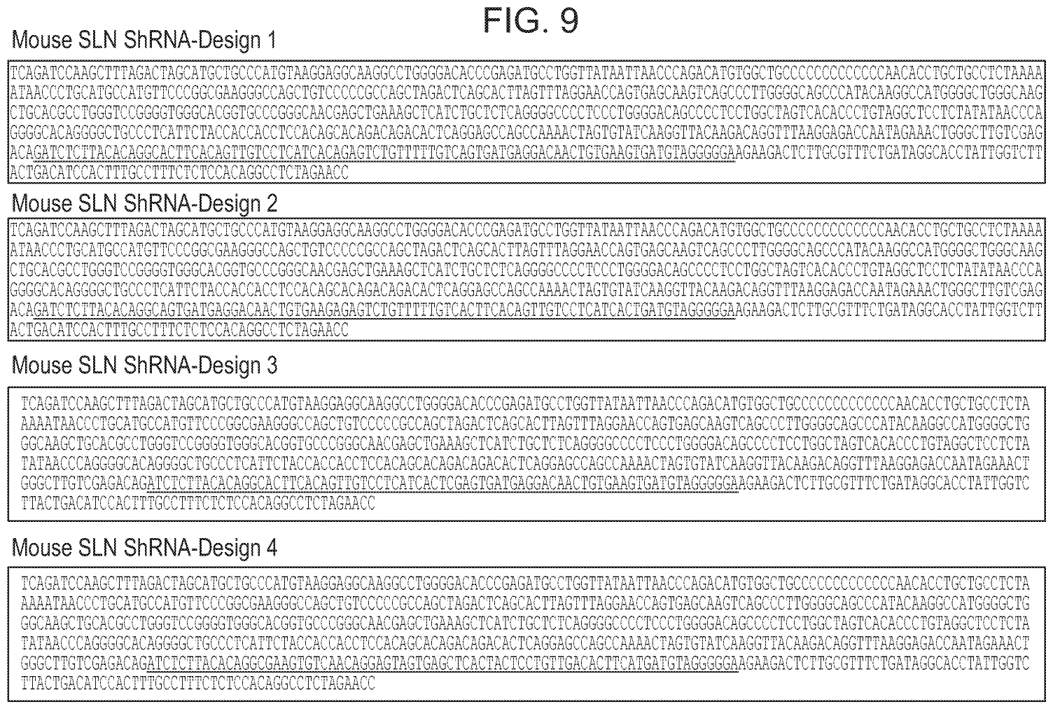

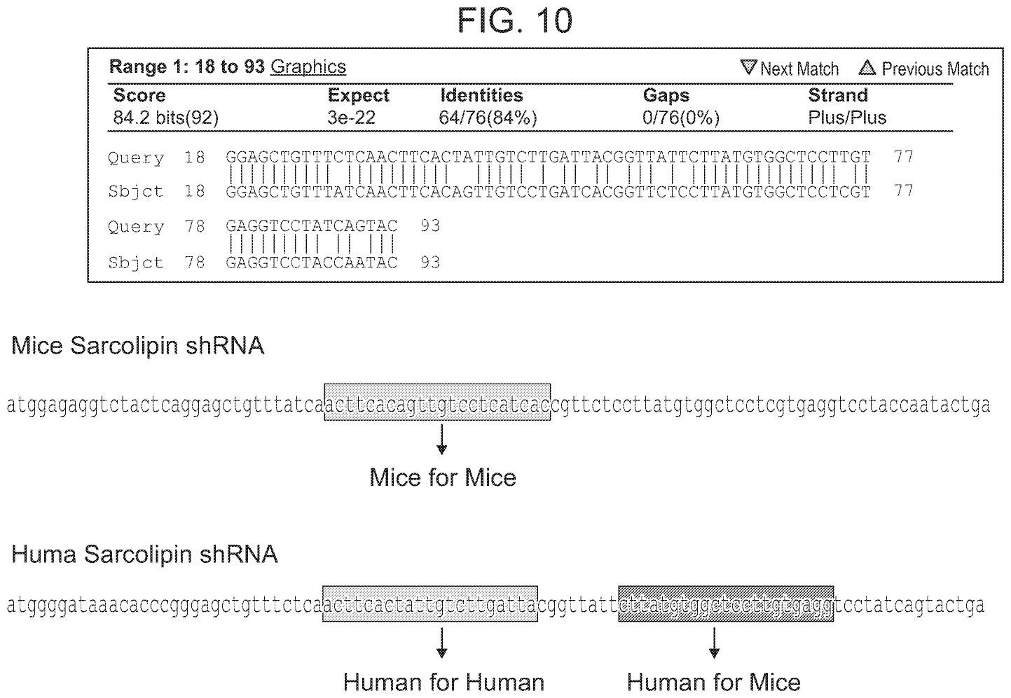

In certain embodiments, the vector of the invention encodes an antisense sequence, or an RNAi sequence (siRNA, shRNA, miRNA etc.), that antagonizes the function of sarcolipin (SLN). In certain embodiments, the vector of the invention encodes an shRNA that antagonizes the function of sarcolipin (shSLN). Exemplary shSLN sequences include those disclosed in FIGS. 9 and 10 (e.g., the underlined sequences in FIG. 9, and the highlighted sequences in FIG. 10). Additional exemplary shSLN sequences include SEQ ID NOs: 7-11 disclosed in WO2018/136880 (incorporated herein by reference).

The invention is also partly directed to gene therapy vectors, e.g., lentiviral or AAV expressing the one or more coding sequence(s), and the dystrophin minigene, as well as methods of delivering the same to the muscle to reduce and/or prevent a secondary cascade symptom while restoring dystrophin function.

In one embodiment, the muscular dystrophy is a congenital muscular dystrophy (CMD) associated with a known genetic defect, such as the fukutin gene or the FKRP (fukutin related protein) gene. Thus in certain embodiments, the congenital muscular dystrophy is Fukuyama congenital muscular dystrophy (FCMD).

Congenital Muscular Dystrophy (CMD) is a group of muscular dystrophies that become apparent at or near birth. In certain embodiments, the methods and rAAV of the invention can be used to treat CMD, especially CMD with known genetic defect in genes such as titin (CMD with cardiomyopathy); SEPN1 (CMD with desmin inclusions, or CMD with (early) spinal rigidity); integrin-alpha 7 (CMD with integrin alpha 7 mutations); integrin-alpha 9 (CMD with joint hyperlaxity); plectin (CMD with familial junctional epidermolysis bullosa); fukutin (Fukuyama CMD or MDDGA4); fukutin-related protein (FKRP) (CMD with muscle hypertrophy or MDC1C); LARGE (MDC1D); DOK7 (CMD with myasthenic syndrome); lamin A/C (CMD with spinal rigidity and lamin A/C abnormality); SBP2 (CMD with spinal rigidity and selenoprotein deficiency); choline kinase beta (CMD with structural abnormalities of mitochondria); laminin alpha 2 (Merosin-deficient CMD or MDC1A); POMGnT1 (Santavuori muscle-eye-brain disease); COLGA1, COL6A2, or COL6A3 (Ullrich CMD); B3GNT1 (Walker-Warburg syndrome: MDDGA type); POMT1 (Walker-Warburg syndrome: MDDGA1 type); POMT2 (Walker-Warburg syndrome: MDDGA2 type); ISPD (MDDGA3, MDDGA4, MDDGB5, MDDGA6, and MDDGA7); GTDC2 (MDDGA8); TMEM5 (MDDGA10); B3GALNT2 (MDDGA11); or SGK196 (MDDGA12).

Thus the lentiviral or rAAV vector of the invention may comprise a polynucleotide encoding any of the wild-type genes defective in the CMD (such as the ones listed herein above), or a functional equivalent thereof, to treat the CMD in a subject in need thereof. The one or more additional coding sequences may encode an RNAi sequence (siRNA, shRNA, miRNA), an antisense sequence, or a microRNA (miRNA) that eliminates or modifies the mutant CMD gene, or a secondary cascade gene up-regulated due to the loss of the wild-type gene function.

For example, Fukuyama congenital muscular dystrophy (FCMD) is due to a mutant FKTN gene, and the one or more additional coding sequences may encode an exon-skipping antisense oligonucleotide to restore correct exon 10 splicing in the defective FKTN gene in the patient.

In another example, the congenital muscular dystrophy is Merosin-deficient congenital muscular dystrophy type 1A (MDC1A) caused by mutations in the 65-exon LAMA2 gene.

Thus the lentiviral or rAAV vector of the invention may comprise a polynucleotide encoding a functional LAMA2 protein. The one or more additional coding sequences may encode an exon-skipping antisense sequence leading to the restored expression of the C-terminal G-domain (exons 45-64), particularly G4 and G5 of LAMA2 that are most important for mediating interaction with α-dystroglycan. For example, exon 4 of the mutant LAMA2 gene may be skipped to treat MDC1A.

In one embodiment, the muscular dystrophy is myotonic dystrophy (DM), such as DM1 or DM2.

Thus the lentiviral or rAAV vector of the invention may comprise a polynucleotide encoding a functional Dystrophia Myotonica Protein Kinase (DMPK) protein defective in DM1, or a functional CCHC-type zinc finger, nucleic acid binding protein gene (CNBP) protein in DM2. The one or more additional coding sequences may encode an RNAi sequence (siRNA, shRNA, miRNA), an antisense sequence, or a microRNA (miRNA) that can be used to target expanded repeats of mutant transcripts in the DMPK gene or the CNBP gene for RNase-mediated degradation. The one or more additional coding sequences may also encode an exon-skipping antisense sequence leading to the skipping of exon 7A in CLCN1 gene in a DM1 patient.

In one embodiment, the muscular dystrophy is Dysferlinopathy caused by mutations in the dysferlin (DYSF) gene, including limb-girdle muscular dystrophy type 2B (LGMD2B) and Miyoshi myopathy (MM).

Thus the lentiviral or rAAV vector of the invention may comprise a polynucleotide encoding a functional DYSF protein defective in LGMD2B or MM. The one or more additional coding sequences may encode an exon-skipping antisense sequence leading to the skipping of exon 32 in a defective DYSF gene in a dysferlinopathy patient.

In one embodiment, the muscular dystrophy is limb-girdle muscular dystrophy (LGMD) caused by mutations in any of the four sarcoglycans genes, namely α (LGMD2D), β (LGMD2E), γ (LGMD2C) and δ (LGMD2F) gene, particularly the γ sarcoglycan (LGMD2C) encoded by the SGCG gene.

Thus the lentiviral or rAAV vector of the invention may comprise a polynucleotide encoding a functional sarcoglycan protein defective in a LGMD, such as the SGCG gene defective in LGMD2C. The one or more additional coding sequences may encode an exon-skipping antisense sequence leading to the skipping of exons 4-7 in a defective LGMD2C gene, such as one with the Δ-521T SGCG mutation.

In one embodiment, the muscular dystrophy is Facioscapulohumeral muscular dystrophy (FSHD) caused by mutations in the DUX4 gene.

Thus the one or more additional coding sequences may encode an RNAi sequence (siRNA, shRNA, miRNA), an antisense sequence, or a microRNA (miRNA) that reduces the expression of DUX4 or a downstream target such as PITX1.

In certain embodiments, the one or more additional coding sequences encode an exon skipping antisense sequence that targets 3′-UTR of DUX4 to reduce its expression. This is because the DUX4 coding sequence is entirely located in the gene first exon, and exon skipping that targets elements in the mRNA 3′ UTR can either disrupt the permissive polyadenylation or interfere with intron 1 or 2 splicing, hence destroying a functional DUX4 mRNA.

Facioscapulohumeral muscular dystrophy (FSHD) is an inherited autosomal dominant disorder characterized clinically by progressive muscle degeneration. It is the third most common muscular dystrophy after Duchenne muscular dystrophy (DMD) and myotonic dystrophy. FSHD is genetically characterized by a pathogenic contraction of a subset of microsatellite repeats on chromosome 4, leading to aberrant expression of the double homeobox protein 4 (DUX4) gene.

There are two types of FSHD: FSHD1 and FSHD2. FSHD1 is the most common form that occurs in over 95% of all FSHD patients. Genetic analysis links FSHD 1 to the genetic contraction of microsatellite D4Z4 repeat array on chromosome 4. FSHD2, on the other hand, has a normal number of D4Z4 repeats but instead involves a heterozygous mutation in the SMCHD1 gene on chromosome 18p, a chromatin modifier. Patients with FSHD1 and FSHD2 share similar clinical presentations.

Current drug therapy does not cure FSHD, but focus on the management of FSHD symptoms, including myostatin inhibitor luspatercept and anti-inflammatory biologics (ATYR1940). The basis for anti-inflammatory biologics is to suppress inflammation commonly seen in muscle pathology of FSHD patients in order to slow phenotype progression. Thus the subject one or more coding sequences may encode an RNAi reagent or antisense RNA against myostatin or an inflammatory pathway gene. Meanwhile, the RNAi reagent such as small interfering RNA (siRNA) and small hairpin RNA (shRNA), or microRNA (miRNA), or antisense oligonucleotides, can be used to knockdown expression of the myopathic DUX4 gene and its downstream molecules including paired-like homeodomain transcription factor 1 (PITX1). Indeed, in vitro studies have demonstrated success in the suppression of DUX4 mRNA expression by administering antisense oligoes into primary skeletal muscle cells of FSHD patients, and by using miRNA against DUX4 delivered to a DUX4 mouse model using AAV vector. In addition, success in the suppression of PITX1 expression has already been demonstrated systemically in vivo.

In certain embodiments, the one or more additional coding sequences can encode the same sequence (e.g., siRNA, shRNA, miRNA, or antisense), and thus the copy number of the additional coding sequence may be regulated or fine tuned for dosing consideration.

In certain embodiments, the one or more additional coding sequences can encode different sequences, either targeting different targets, or targeting the same target. For example, in certain embodiments, one additional coding sequence is an antisense against a target, while another additional coding sequence is an shRNA against the same target. Alternatively, two additional coding sequences are both shRNAs but they target different regions of the same target.

In certain embodiments, expression of the functional protein, such as the dystrophin minigene product, is not negatively affected by the insertion of the one or more coding sequence(s).

By early 1990s, it has been found that many intronless transgenes, while express perfectly in tissue culture cells in vitro, fail to express the same transgene in vivo (e.g., in transgenic mice harboring the transgene), while inserting certain heterologous intron sequences between the promoter and the (intronless) coding sequence of the transgene greatly enhances transgene expression in vivo.

In particular, Palmiter et al. (Proc. Natl. Acad. Sci. U.S.A. 88:478-482, 1991, incorporated herein by reference) showed that several heterologous introns inserted between the metallothionein promoter and the growth hormone transgene improves transgene expression, and provided addition of certain heterologous introns as a general strategy for improving transgene expression. These include heterologous introns selected from: the natural first intron of rGH, intron A of the rat insulin II (rIns-II) gene, intron B of the hβG gene, and the SV40 small t intron.

A similar finding was confirmed by Choi et al. (Mol. Cell. Biol. 11(6):3070-3074, 1991, incorporated herein by reference), who reported that in transgenic mice carrying the human histone H4 promoter linked to the bacterial gene for chloramphenicol acetyltransferase (CAT), the presence of a 230-bp heterologous hybrid intron in the transcription unit greatly enhanced CAT activity (by 5- to 300-fold, compared to an analogous transgene precisely deleted for the intervening sequences). This hybrid intron, consisting of an adenovirus splice donor and an immunoglobulin G splice acceptor, stimulated expression in a broad range of tissues in the animal. Since the hybrid intron stimulated the expression of tissue plasminogen activator and factor VIII in tissue culture, Choi concluded that the enhancement seen in mice is unlikely to be specific to CAT and instead is generally applicable to the expression of any cDNAs in transgenic mice.

Thus in certain embodiment, the heterologous intron in the subject lentiviral or rAAV vector is selected from the group consisting of: the natural first intron of rGH, intron A of the rat insulin II (rIns-II) gene, intron B of the hβG gene, the SV40 small t intron, and the hybrid intron of Choi.

In certain embodiments, the heterologous intron sequence is SEQ ID NO: 1:

GTATCAAGGTTACAAGACAGGTTTAAGGAGACCAATAGAAACTGGGCTTG
TCGAGACAGAGAAGACTCTTGCGTTTCTGATAGGCACCTATTGGTCTTAC
TGACATCCACTTTGCCTTTCTCTCCACAG.

In certain embodiments, the one or more additional coding sequences are all inserted into the heterologous intron sequence (SEQ ID NO: 1), or all inserted into the 3′-UTR region, or are inserted into both regions. For example, the microRNA-29c coding sequence can be inserted into the intron coding sequence as in SEQ ID NO: 2

GTATCAAGGTTACAAGACAGGTTTAAGGAGACCAATAGAAACTGGGCTTG
TCGAGACAGATCTCTTACACAGGCTGACCGATTTCTCCTGGTGTTCAGAG
TCTGTTTTTGTCTAGCACCATTTGAAATCGGTTATGATGTAGGGGGAAGA
AGACTCTTGCGTTTCTGATAGGCACCTATTGGTCTTACTGACATCCACTT
TGCCTTTCTCTCCACAG.

The miR-29c sequence in SEQ ID NO: 2 is

(SEQ ID NO: 3)
ATCTCTTACACAGGCTGACCGATTTCTCCTGGTGTTCAGAGTCTGTTTTT
GTCTAGCACCATTTGAAATCGGTTATGATGTAGGGGGA.

In certain embodiments, the lentiviral or rAAV further comprises two lentiviral or AAV LTR/ITR sequences flanking the polynucleotide (such as the dystrophin minigene) and the additional coding sequence(s).

In certain embodiments, the lentiviral or rAAV vectors of the invention may be operably linked to a muscle-specific control element. For example, the muscle-specific control element can be: human skeletal actin gene element, cardiac actin gene element, myocyte-specific enhancer binding factor MEF, muscle creatine kinase (MCK), tMCK (truncated MCK), myosin heavy chain (MHC), C5-12 (synthetic promoter), murine creatine kinase enhancer element, skeletal fast-twitch troponin C gene element, slow-twitch cardiac troponin C gene element, slow-twitch troponin I gene element, hypozia-inducible nuclear factors, steroid-inducible element, or glucocorticoid response element (GRE).

In certain embodiments, muscle-specific control element is 5′ to the heterologous intron sequence, which is 5′ to the dystrophin minigene, which comprises a 3′-UTR region including a translation stop codon (such as TAG), a polyA adenylation signal (such as AATAAA), and an mRNA cleavage site (such as CA).

In certain embodiments, the muscle-specific control element comprises the nucleotide sequence of SEQ ID NO: 10 or SEQ ID NO: 11 of WO2017/181015.

| SEQ ID NO: 10 of WO2017/181015: | |
|---|---|
| CAGCCACTAT GGGTCTAGGC TGCCCATGTA AGGAGGCAAG GCCTGGGGAC ACCCGAGATG | 60 |
| CCTGGTTATA ATTAACCCAG ACATGTGGCT GCTCCCCCCC CCCAACACCT GCTGCCTGAG | 120 |
| CCTCACCCCC ACCCCGGTGC CTGGGTCTTA GGCTCTGTAC ACCATGGAGG AGAAGCTCGC | 180 |
| TCTAAAAATA ACCCTGTCCC TGGTGG | 206 |
| SEQ ID NO: 11 of WO2017/181015: | |
| GCTGTGGGGG ACTGAGGGCA GGCTGTAACA GGCTTGGGGG CCAGGGCTTA TACGTGCCTG | 60 |
| GGACTCCCAA AGTATTACTG TTCCATGTTC CCGGCGAAGG GCCAGCTGTC CCCCGCCAGC | 120 |
| TAGACTCAGC ACTTAGTTTA GGAACCAGTG AGCAAGTCAG CCCTTGGGGC AGCCCATACA | 180 |
| AGGCCATGGG GCTGGGCAAG CTGCACGCCT GGGTCCGGGG TGGGCACGGT GCCCGGGCAA | 240 |
| CGAGCTGAAA GCTCATCTGC TCTCAGGGGC CCCTCCCTGG GGACAGCCCC TCCTGGCTAG | 300 |
| TCACACCCTG TAGGCTCCTC TATATAACCC AGGGGCACAG GGGCTGCCCC CGGGTCAC | 358 |

In certain embodiments, the rAAV vectors of the invention can be operably linked to the muscle-specific control element comprising the MCK enhancer nucleotide sequence (see SEQ ID NO: 10 of WO2017/181015, incorporated herein by reference) and/or the MCK promoter sequence (see SEQ ID NO: 11 of WO2017/181015, incorporated herein by reference).

In certain embodiments, the rAAV further comprises a promoter operably linked to and is capable of driving the transcription of the dystrophin minigene and the additional coding sequence.

An exemplary promoter is the CMV promoter.

In certain embodiments, the rAAV further comprises a poly-A adenylation sequence for inserting a polyA sequence into a transcribed mRNA.

In certain embodiments, the rAAV vectors of the invention are of the serotype AAV1, AAV2, AAV4, AAV5, AAV6, AAV7, AAVrh.74, AAV8, AAV9, AAV10, AAV11, AAV12 or AAV13.

Another aspect of the invention provides a method of producing a viral vector, e.g., rAAV vector of the invention, comprising culturing a cell that has been transfected with any viral vector, e.g., rAAV vector of the invention and recovering the virus, e.g., rAAV particles from the supernatant of the transfected cells.

Another aspect of the invention provides viral particles comprising any of the viral vector, e.g., recombinant AAV vectors of the invention.

Another aspect of the invention provides methods of producing a functional protein either defective in a muscular dystrophy, or effective to treat the muscular dystrophy (such as a micro-dystrophin protein), and one or more additional coding sequence(s), comprising infecting a host cell with a subject recombinant AAV vector co-expressing the functional protein (e.g., micro-dystrophin) of the invention and the coding sequence product (e.g., RNAi, siRNA, shRNA, miRNA, antisense, microRNA or inhibitor thereof) in the host cell.

Another aspect of the invention provides methods of treating a muscular dystrophy (such as DMD or BMD) or dystrophinopathy in a subject in need thereof, the method comprising administering to the subject a therapeutically effective amount of a viral vector, e.g., recombinant AAV vector of the invention, or a composition of the invention.

The invention contemplates administering any of the viral vector, e.g., AAV vectors of the invention to patients diagnosed with dystrophinopathy or muscular dystrophy, such as DMD or BMD or any other MD, particularly defective dystrophin-associated muscular dystrophy, preferably before one or more secondary cascade symptoms such as fibrosis is observed in the subject, or before the muscle force has been reduced in the subject, or before the muscle mass has been reduced in the subject.

The invention also contemplates administering any of the viral vector, e.g., rAAV of the invention to a subject suffering from dystrophinopathy or muscular dystrophy, such as DMD or BMD or any other MD, particularly dystrophin-associated muscular dystrophy, who already has developed one or more secondary cascade symptoms such as fibrosis, in order to prevent or slow down further disease progression in these subjects.

Another aspect of the invention provides recombinant viral vector, e.g., AAV vectors comprising a nucleotide sequence encoding a functional protein either defective in a muscular dystrophy, or effective to treat the muscular dystrophy (e.g., a micro-dystrophin protein) and the one or more additional coding sequences.

In certain embodiments, the invention provides for a rAAV comprising a nucleotide sequence having at least 85%, 90%, 95%, 97%, or 99% identity to the nucleotide sequence that encodes a functional micro-dystrophin protein such as microD5.

The viral vector, e.g., rAAV vector may comprise a muscle-specific promoter, such as the MCK promoter, a heterologous intron sequence effective to enhance the expression of the dystrophin gene, the coding sequence for the micro-dystrophin gene, polyA adenylation signal sequence, the ITR/LTR repeats flanking these sequences. The viral vector, e.g., rAAV vector may optionally further comprises ampicillin resistance and plasmid backbone sequences or pBR322 origin or replication for amplification in a bacteria host.

In one aspect, the recombinant AAV vectors of the invention are AAV1, AAV2, AAV4, AAV5, AAV6, AAV7, AAVrh.74, AAV8, AAV9, AAV10, AAV 11, AAV 12 or AAV 13.

In any of the methods of the invention, the rAAV vector can be administered by intramuscular injection or intravenous injection.

In any of the methods of the invention, the viral vector, e.g., rAAV vector or composition is administered systemically. For examples, the viral vector, e.g., rAAV vector or composition is parentally administration by injection, infusion or implantation.

Another aspect of the invention provides a composition, such as a pharmaceutical composition, comprising any of the viral vector, e.g., rAAV vectors of the invention.

In certain embodiments, the composition is a pharmaceutical composition, which may further comprise a therapeutically compatible carrier or excipient.

In another embodiment, the invention provides for composition comprising any of the viral vector, e.g., rAAV vectors co-expressing the subject functional protein (e.g., micro-dystrophin) and said one or more additional coding sequences for treating a subject suffering from dystrophinopathy or a muscular dystrophy, such as DMD or Becker Muscular dystrophy.

The compositions (e.g., pharmaceutical compositions) of the invention can be formulated for intramuscular injection or intravenous injection. The composition of the invention can also be formulated for systemic administration, such as parentally administration by injection, infusion or implantation. In addition, any of the compositions are formulated for administration to a subject suffering from dystrophinopathy or a muscular dystrophy, such as DMD, Becker muscular dystrophy or any other dystrophin associated muscular dystrophy.

In a further embodiment, the invention provides for use of any of the viral vector, e.g., rAAV vectors of the invention co-expressing a subject functional protein (e.g., a micro-dystrophin) and said one or more additional coding sequences for preparation of a medicament for reducing the subject suffering from dystrophinopathy or muscular dystrophy, such as DMD, Becker muscular dystrophy or any other dystrophin associated muscular dystrophy.

The invention contemplates use of the any of the viral vector, e.g., AAV vectors of the invention for the preparation of a medicament for administration to a patient diagnosed with DMD before one or more secondary cascade symptoms such as fibrosis is observed in the subject.

The invention also contemplates use of any of the viral vector, e.g., AAV vectors of the invention for the preparation of a medicament for administering any of the viral vector, e.g., rAAV of the invention to a subject suffering from muscular dystrophy who already has developed a secondary cascade symptom such as fibrosis, in order to prevent or delay disease progression in these subjects.

The invention also provides for use of the viral vector, e.g., rAAV vectors of the invention co-expressing a subject functional protein such as a micro-dystrophin, and said one or more additional coding sequences for the preparation of a medicament for treatment of a muscular dystrophy, such as DMD/BMD.

In any of the uses of the invention, the medicament can be formulated for intramuscular injection. In addition, any of the medicaments may be prepared for administration to a subject suffering from muscular dystrophy such as DMD or any other dystrophin associated muscular dystrophy.

The present invention also provides for gene therapy vectors, e.g., rAAV vectors that co-express a subject functional protein (e.g., a micro-dystrophin) and said one or more additional coding sequences in a muscular dystrophy patient.

It should be understand that any one embodiment of the invention described herein can be combined with any one or more additional embodiments of the invention, including those embodiments described only in the examples or only described in one of the sections above or below, or one aspect of the invention.

### AAV

As used herein, the term “AAV” is a standard abbreviation for adeno-associated virus. Adeno-associated virus is a single-stranded DNA parvovirus that grows only in cells in which certain functions are provided by a co-infecting helper virus. There are at least thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York) (incorporated herein by reference). However, it is fully expected that these same principles will be applicable to additional AAV serotypes since it is well known that the various serotypes are quite closely related, both structurally and functionally, even at the genetic level. See, for example, Blacklowe, 1988, pp. 165-174 of Parvoviruses and Human Disease, J. R. Pattison, ed.; and Rose, Comprehensive Virology 3: 1-61 (1974). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

Recombinant AAV genomes of the invention comprise nucleic acid molecule of the invention and one or more AAV ITRs flanking a nucleic acid molecule.

There are multiple serotypes of AAV, and the nucleotide sequences of the genomes of the AAV serotypes are known. For example, the nucleotide sequence of the AAV serotype 2 (AAV2) genome is presented in Srivastava et al., J Virol 45:555-564 (1983) as corrected by Ruffing et al., J Gen Virol 75:3385-3392 (1994). Both incorporated herein by reference. As other examples, the complete genome of AAV-1 is provided in GenBank Accession No. NC_002077 (incorporated herein by reference); the complete genome of AAV-3 is provided in GenBank Accession No. NC_001829 (incorporated herein by reference); the complete genome of AAV-4 is provided in GenBank Accession No. NC_001829 (incorporated herein by reference); the AAV-5 genome is provided in GenBank Accession No. AF085716 (incorporated herein by reference); the complete genome of AAV-6 is provided in GenBank Accession No. NC_001862 (incorporated herein by reference); at least portions of AAV-7 and AAV-8 genomes are provided in GenBank Accession Nos. AX753246 (incorporated herein by reference) and AX753249 (incorporated herein by reference), respectively (see also U.S. Pat. Nos. 7,282,199 and 7,790,449 relating to AAV-8); the AAV-9 genome is provided in Gao et al., J. Virol 78:6381-6388 (2004), incorporated herein by reference; the AAV-10 genome is provided in Mol. Ther. 13(1):67-76 (2006), incorporated herein by reference; and the AAV-11 genome is provided in Virology 330(2):375-383 (2004), incorporated herein by reference. The AAVrh74 serotype is described in Rodino-Klapac et al., J. Trans. Med. 5:45 (2007), incorporated herein by reference.

AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, Rh10, Rh74, and AAV-2i8.

Production of pseudotyped rAAV is disclosed in, for example, WO 01/83692 which is incorporated by reference herein in its entirety.

Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art.

In certain embodiments, to promote skeletal muscle specific expression, AAV1, AAV6, AAV8 or AAVrh.74 may be used.

In certain embodiments, the AAV serotype of the subject AAV vector is AAV9.

Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes.

The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and it encodes the three capsid proteins VP1, VP2, and VP3. Alternative splicing and non-consensus translational start sites are responsible for the production of the three related capsid proteins.

A single consensus polyadenylation site is located at map position 95 of the AAV genome. The life cycle and genetics of AAV are reviewed in Muzyczka, Current Topics in Microbiology and Immunology 158:97-129 (1992).

DNA plasmids of the invention comprise rAAV genomes of the invention. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell, are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAVrh.74, AAV-8, AAV-9, AAV-10, AAV-11, AAV-12 and AAV-13.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., Proc. Natl. Acad. Sci. U.S.A. 79:2077-2081, 1982), addition of synthetic linkers containing restriction endonuclease cleavage sites (Laughlin et al., Gene 23:65-73, 1983) or by direct, blunt-end ligation (Senapathy & Carter, J. Biol. Chem. 259:4661-4666, 1984). The packaging cell line is then infected with a helper virus such as adenovirus. The advantages of this method are that the cells are selectable and are suitable for large-scale production of rAAV.

Other examples of suitable methods employ adenovirus or baculovirus rather than plasmids to introduce rAAV genomes and/or rep and cap genes into packaging cells.

General principles of rAAV production are reviewed in, for example, Carter, Current Opinions in Biotechnology 1533-1539, 1992; and Muzyczka, Curr. Topics in Microbial. and Immunol. 158:97-129, 1992). Various approaches are described in Ratschin et al., Mol. Cell. Biol. 4:2072, 1984; Hermonat et al., Proc. Natl. Acad. Sci. U.S.A. 81:6466, 1984; Tratschin et al., Mol. Cell. Biol. 5:3251, 1985; McLaughlin et al., J. Virol. 62: 1963, 1988; and Lebkowski et al., Mol. Cell. Biol. 7:349, 1988; Samulski et al., J. Virol. 63:3822-3828, 1989; U.S. Pat. No. 5,173,414; WO 95/13365, and corresponding U.S. Pat. No. 5,658,776; WO95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine 13:1244-1250, 1995; Paul et al., Human Gene Therapy 4:609-615, 1993; Clark et al., Gene Therapy 3:1124-1132, 1996; U.S. Pat. Nos. 5,786,211; 5,871,982; and 6,258,595. The foregoing documents are hereby incorporated by reference in their entirety herein, with particular emphasis on those sections of the documents relating to rAAV production.

In certain embodiments, the AAV vectors of the invention are produced according to the method described in Adamson-Small et al. (Molecular Therapy—Methods & Clinical Development (2016) 3, 16031; doi:10.1038/mtm.2016.31, incorporated herein by reference), a scalable method for the production of high-titer and high quality adeno-associated type 9 vectors using the HSV platform. It is a complete herpes simplex virus (HSV)-based production and purification process capable of generating greater than 1×10¹⁴ rAAV9 vector genomes per 10-layer CellSTACK of HEK 293 producer cells, or greater than 1×10⁵ vector genome per cell, in a final, fully purified product. This represents a 5- to 10-fold increase over transfection-based methods. In addition, rAAV vectors produced by this method demonstrated improved biological characteristics when compared to transfection-based production, including increased infectivity as shown by higher transducing unit-to-vector genome ratios and decreased total capsid protein amounts, shown by lower empty-to-full ratios. This method can also be readily adapted to large-scale good laboratory practice (GLP) and good manufacturing practice (GMP) production of rAAV9 vectors to enable preclinical and clinical studies and provide a platform to build on toward late-phases and commercial production. Although AAV9 was used in the study, this method is likely extendable to other serotypes and should bridge the gap between preclinical research, early phase clinical studies, and large-scale, worldwide development of gene therapy based-drugs for genetic diseases and disorders.

The invention thus provides packaging cells that produce infectious rAAV. In one embodiment, packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (i.e., infectious encapsidated rAAV particles) of the invention comprise a rAAV genome. In exemplary embodiments, the genomes of both rAAV lack AAV rep and cap DNA, that is, there is no AAV rep or cap DNA between the ITRs of the genomes. Examples of rAAV that may be constructed to comprise the nucleic acid molecules of the invention are set out in International Patent Application No. PCT/US2012/047999 (WO 2013/016352) incorporated by reference herein in its entirety.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther. 10(6):1031-1039, 1999; Schenpp and Clark, Methods Mol. Med. 69:427-443, 2002; U.S. Pat. No. 6,566,118 and WO 98/09657.

### Additional Coding Sequences

In addition to the coding sequence for a dystrophin protein, such as microD5, the recombinant vector of the invention also comprises one or more additional coding sequences for targeting gene(s) in one of the secondary complications/secondary cascades associated with or resulting from loss of dystrophin.

In certain embodiments, the vector of the invention encodes an exon-skipping antisense sequence that can correct specific dystrophin gene mutations.

For example, the exon-skipping antisense sequence induces skipping of specific exons during pre-messenger RNA (pre-mRNA) splicing of a defective dystrophin gene in the subject, resulting in restoration of the reading frame and partial production of an internally truncated protein, similar to the dystrophin protein expression seen in Becker muscular dystrophy.

In certain embodiments, the exon-skipping antisense sequence skips or splices out a frame-disrupting exon (mutated exon) and/or a neighboring exon to restore the correct transcriptional reading frame, and to produce a shorter but functional dystrophin protein.

In certain embodiments, the exon-skipping antisense sequence induces single exon skipping. In certain embodiments, the exon-skipping antisense sequence induces multiple exon skipping, such as skipping of one or more of, or all of exons 45-55 (i.e., native exons 44 is joined directly to exon 56). For example, 11 antisense sequences may be used together to skip all 11 exons including exons 45-55. A cocktail of 10 AONs was used in the mdx52 mouse model (with deletion of exon 52) to induce skipping of exon 45-51 and 53-55, thus restoring functional dystrophin expression.

In certain embodiments, the exon-skipping antisense sequence induces skipping of exon 51 in a dystrophin pre-mRNA. Successful skipping of exon 51 can in theory treat about 14% of all DMD patients.

In certain embodiments, the exon-skipping antisense sequence targets an exonic splice enhancer (ESE) site in exon 51 of dystrophin gene, thus causing a skip of exon 51 and producing a truncated but partially functional dystrophin protein.

In certain embodiments, the exon-skipping antisense sequence induces skipping of one or more of exons 44, 45, and 53.

In certain embodiments, the exon-skipping antisense sequence targets the same target sequence as that of casimersen (exon 45), NS-065/NCNP-01 or golodirsen (exon 53), or eteplirsen or Exondys 51 (exon 51).

In certain embodiments, the exon-skipping antisense sequence targets a cryptic splicing donor and/or acceptor site in the mutated FCMD/FKTN gene in a Fukuyama congenital muscular dystrophy (FCMD) patient to restore correct exon 10 splicing.

Fukuyama congenital muscular dystrophy (FCMD) is a rare autosomal recessive disease and the second prevalent form of childhood muscular dystrophy in Japan. The gene responsible for FCMD (FCMD, also known as FKTN) encodes the protein fukutin, which is a putative glycosyltransferase and glycosylates α-dystroglycan, a member of the dystrophin-associated glycoprotein complex (DAGC). The pathogenesis of FCMD is caused by an ancestral insertion of SINE-VNTR-Alu(SVA) retrotransposon into the 3′-untranslated region (UTR) of the fukutin gene, leading to the activation of a new, cryptic splice donor in exon 10, and a new, cryptic splice acceptor in the SVA insertion site, thus inducing aberrant mRNA splicing between the cryptic donor and acceptor sites. The result is a premature truncation of exon 10 of FCMD. In FCMD patient cells and model mice in vivo, it has been shown that a cocktail of three vivo-PMOs targeting the cryptic splice modulating regions prevented pathogenic SVA exon trapping and restored normal FKTN protein levels and O-glycosylation of α-dystroglycan.

In certain embodiments, the antisense sequence targets a pathological expansion of 3- or 4-nucleotide repeats, such as a CTC triplet repeat in the 3′-UTR region of the DMPK gene in DM1 patients, or a CCTG repeat in the first intron of the CNBP gene in DM2 patients.

Myotonic dystrophy (DM) is the most common form of muscular dystrophy in adulthood. It is an autosomal dominant disease that can be categorized into myotonic dystrophy type 1 (DM1) and myotonic dystrophy type 2 (DM2). DM1 is caused by a pathological expansion of CTC triplet in 3′-UTR region of the Dystrophia Myotonica Protein Kinase (DMPK) gene, while DM2 is caused by a pathological expansion of CCTG tract in the first intron of the CCHC-type zinc finger, nucleic acid binding protein gene (CNBP). RNA gain-of-function toxicity, arising from transcribed RNA aggregates with expanded repeats, leads to aberrant splicing (spliceopathy). Aggregates of toxic RNA disrupt the function of alternative splicing regulators such as Muscleblind-like (MBNL) protein and CUG-binding protein 1 (CUGBP1), by sequestering and depleting the former within the nuclear RNA foci, and increasing the expression and phosphorylation of the latter in DM1. Alterations in the function of MBNL and CUGBP1 proteins lead to aberrant splicing in pre-mRNAs of target genes, namely insulin receptor (INSR), the muscle chloride channel (CLCN1), bridging integrator-1 (BIN1), and dystrophin (DMD), which are respectively associated with insulin resistance, myotonia, muscle weakness, and dystrophic muscle processes (all typical symptoms of myotonic dystrophy).

Thus an expanded CUG repeat in the DMPK gene sequesters MBNL1 protein and causes aberrant splicing in several downstream genes, thereby causing DM1 phenotype. Meanwhile, antisense oligonucleotides can be used to target such expanded repeats of mutant transcripts for RNase-mediated degradation, thereby restoring splicing of downstream genes. A 2′-O-methoxyethyl gapmer AON has been used to target the degradation of expanded CUG by RNase H in mutant RNA transcripts, resulting in a reduction of mutant mRNA transcripts and restored protein expression.

In certain embodiments, the exon-skipping antisense sequence leads to skipping of exon 7A in CLCN1 gene in a DM1 patient.

DM1 can also be treated by correcting aberrant splicing of chloride channel 1 (CLCN1), as this gene causes myotonia in DM1 patients. Using PMOs (phosphorodiamidate morpholino oligomer) with bubble liposomes through ultrasound exposure to enhance delivery of PMOs into muscles of DM1 mice (HSALR), skipping of exon 7A of CLCN1 was achieved in vivo, resulting in ameliorated myotonia and Clcn1 protein expression in skeletal muscles.

In certain embodiments, the exon-skipping antisense sequence targets exons 17, 32, 35, 36, and/or 42 of the DYSF gene, preferably exon 32 and/or 36, for exon skipping in a dysferlinopathy (e.g., LGMD2B or MM) patient with a DYSF mutation.

Dysferlinopathy is an umbrella term that encompasses muscular dystrophies caused by mutations in the dysferlin (DYSF) gene. Dysferlin gene encodes a sarcolemmal protein required for repairing muscle membrane damage. It consists of calcium-dependent C2 lipid binding domains and a vital transmembrane domain. There are two common dysferlinopathies—limb-girdle muscular dystrophy type 2B (LGMD2B) and Miyoshi myopathy (MM), both have clinically distinct phenotypes and an autosomal recessive inheritance. LGMD2B is characterized by proximal muscle weakness, while MM is characterized by distal muscle weakness. Initial clinical phenotypes of LGMD2B and MM are distinct. However, as the disease progresses, the clinical presentations for both conditions overlap, becoming more similar, and patients experience muscle weakness in both proximal and distal limbs. Dysferlin-deficient muscle fibers have a defect in membrane repair.

Dysferlinopathies can be treated by exon skipping using antisense oligonucleotides, partly due to the observed mild phenotype in a patient with only 10% wild-type level expression of a truncated mutant DYSF protein. Specifically, in an LGMD2B case of a compound heterozygous female patient, the patient harbored one null allele and a DYSF branch point mutation on the other allele in intron 31. A natural in-frame skipping of exon 32 resulted in a truncated dysferlin protein expressed at about 10% that of the wild type levels, which was sufficient to partially complement the null mutation. The patient exhibited mild symptoms, and was ambulant at age 70. Recently, it has been shown that exon 32 skipping in patient cells resulted in quasi-dysferlin expression levels, which rescued membrane repair in treated cells that were subject to hypo-osmotic pressure and sarcolemmal localized laser injury in vitro.

In certain embodiments, the exon-skipping antisense sequence targets exon 4 of the LAMA2 gene, for exon skipping in a merosin-deficient congenital muscular dystrophy type 1A (MDC1A) patient with a LAMA2 mutation. In certain embodiments, exon skipping results in restored expression of the C-terminal G-domain (exons 45-64), particularly G4 and G5 that are most important for mediating interaction with α-dystroglycan.

Merosin-deficient congenital muscular dystrophy type 1A (MDC1A) is caused by mutations in the 65-exon LAMA2 gene that results in a complete or partial deficiency in laminin-α2 chain expression. Laminin-α2 chain, together with beta1 (β1), and gamma1 (γ1) chains, are parts of the heterotrimeric laminin isoform known as Laminin-211 or merosin, which is expressed particularly in the basement membranes of skeletal muscles, including the neuromuscular junction and Schwann cells (peripheral nerves). Laminin-α2 interacts with the dystrophin—dystroglycan complex (DGC), mediating cell signaling, adhesion, and tissue integrity in skeletal muscles and peripheral nerves. Although not always the case, the partial expression of laminin-α2 causes milder MDC1A, while complete absence of laminin-α2 causes severe MDC1A. The C-terminal G-domain (exons 45-64), particularly G4 and G5, are most important for mediating interaction with α-dystroglycan. Mutations eliminating G4 and G5 is associated with severe phenotypes even in the presence of partial truncated laminin-α2 expression.

Exon-skipping has been explored for treating MDC1A, in that PMO-mediated exon 4 skipping corrected the open reading frame, resulting in the recovery of a truncated laminin-α2 chain and a slightly extended patient life span.

In certain embodiments, the exon-skipping antisense sequence induces skipping of exons 4-7 of the most common Δ-521T mutation in the LGMD2C/SGCG gene, and restoration of the reading frame to generate an internally truncated SGCG protein, for treating a limb-girdle muscular dystrophy type 2C patient with a Δ-521T SGCG mutation. In certain embodiments, exon skipping results in restored expression of the internally truncated SGCG protein that retains the intracellular, transmembrane, and extreme carboxy-terminus of the wild-type SGCG protein.

Dystrophin-associated protein (DAP) is a complex in the muscle cell membrane, the transmembrane components of which link the cytoskeleton to the extracellular matrix in adult muscle fibers, and are essential for the preservation of the integrity of the muscle cell membrane. The sarcoglycan subcomplex within the DGC is composed of 4 single-pass transmembrane subunits: α-, β-, γ-, and δ-sarcoglycan. The α to δ-sarcoglycans gene, namely α (LGMD2D), β (LGMD2E), γ (LGMD2C) and δ (LGMD2F), are expressed predominantly (β) or exclusively (α, γ and δ) in striated muscle. A mutation in any of the four sarcoglycan genes may lead to a secondary deficiency of the other sarcoglycan proteins, presumably due to destabilization of the sarcoglycan complex, leading to sarcoglycanopathies—autosomal recessive limb girdle muscular dystrophies (LGMDs). The disease-causing mutations in the α to δ genes cause disruptions within the dystrophin-associated protein (DAP) complex in the muscle cell membrane.

In human, γ sarcoglycan (LGMD2C) is a protein encoded by the SGCG gene. Severe childhood autosomal recessive muscular dystrophy (SCARMD) is a progressive muscle-wasting disorder that segregates with microsatellite markers at the γ-sarcoglycan gene. Mutations in the γ-sarcoglycan gene were first described in the Maghreb countries of North Africa, where γ-sarcoglycanopathy has a higher than usual incidence. One of the most common mutation in LGMD 2C patients, Δ-521T, is a deletion of a thymine from a string of 5 thymines at nucleotide bases 521-525 in exon 6 of the γ-sarcoglycan gene. This mutation shifts the reading frame and results in the absence of γ-sarcoglycan protein and secondary reduction of β- and δ-sarcoglycans, thus causing a severe phenotype. The mutation occurs both in the Maghreb population and in other countries.

Exon-skipping has been explored for treating LGMD2C with the Δ-521T mutation, in that the resulting internally truncated SGCG protein provided functional and pathological benefits to correct the loss of γ-sarcoglycan in a Drosophila model, in heterologous cell expression studies, and in transgenic mice lacking γ-sarcoglycan. A cellular model of human muscle disease was also generated to show that multiple exon skipping could be induced in RNA that encodes a mutant human γ-sarcoglycan.

In certain embodiments, the vector of the invention encodes an antisense sequence, or an RNAi sequence (siRNA, shRNA, miRNA etc.), that antagonizes the function of sarcolipin (SLN). In certain embodiments, the vector of the invention encodes an shRNA that antagonizes the function of sarcolipin (shSLN).

Exemplary shSLN sequences include those disclosed in FIGS. 9 and 10 (e.g., the underlined sequences in FIG. 9, and the highlighted sequences in FIG. 10). Additional exemplary shSLN sequences include SEQ ID NOs: 7-11 disclosed in WO2018/136880 (incorporated herein by reference).

Further shSLN sequences can be designed based on any art recognized methods, using the human SLN mRNA sequence shown below.

| (SEQ ID NO: 4) | |
|---|---|
| 1 | AGACAGCCTG GGAGGGGAGA AGGAGTTGGA GCTCAAGTTG GAGACAGCGA GGAGAAACCT |
| 61 | GCCATAGCCA GGGTGTGTCT TTGATCCTCT TCAGGAGGTG AGGAGAAGCC AGAGGTCCTT |
| 121 | GGTGTGCCCT CAGAAATCTG CCTGCAGTTC TCACCAAGCC GCTGTGAAAA TGGGGATAAA |
| 181 | CACCCGGGAG CTGTTTCTCA ACTTCACTAT TGTCTTGATT ACGGTTATTC TTATGTGGCT |
| 241 | CCTTGTGAGG TCCTATCAGT ACTGAGAGGC CATGCCATGG TCCTGGGATT GACTGAGATG |
| 301 | CTCCGGAGCT GCCTGCTCTA TGCCCTGAGA CCCCACTGCT GTCATTGTCA CAGGATGCCA |
| 361 | TTCTCCATCC GAGGGCACCT GTGACCTGCA CTCACAATAT CTGCTATGCT GTAGTGCTAG |
| 421 | GATTGATTAT GTGTTCTCCA AAGATGCTGC TCCCAAGGGC TGCCAAGTGT TTGCCAGGGA |
| 481 | ACGGTAGATT TATTCCCCAA CTCTTAACTG AAAATGTGTT AGACAAGCCA CAAAGTTAAA |
| 541 | ATTAAACTGG ATTCATGATG ATGTAGGATT GTTACAAGCC CCTGATCTGT CTCACCACAC |
| 601 | ATCCCTTCAA CCCACACGGT CTGCAACCAA ACTCTAATTC AACCTGCCAG AAGGAATGTT |
| 661 | AGAGGAAGTC TTTGTCAGCC CTTATAGCTA TCATGTGAAT AAAGTTAAGT CAACTTCAAA |
| 721 | AA |

In certain embodiments, the vector of the invention encodes an antisense sequence, or an RNAi sequence (siRNA, shRNA, miRNA etc.), that antagonizes the function of one or more target genes, such as an inflammatory gene.

The IκB kinase/nuclear factor-kappa B (NF-κB) signaling is persistently elevated in immune cells and regenerative muscle fibers in both animal models and patients with DMD. In addition, activators of NF-κB such as TNF-α and IL-1 and IL-6 are upregulated in DMD muscles. Thus, inhibiting the NF-κB signaling cascade components, such as NF-κB itself, its upstream activators and the downstream inflammatory cytokines, are beneficial for treating the subject patients in conjunction with replacing/repairing a defective dystrophin gene.

Thus in certain embodiments, the vector of the invention encodes an antisense sequence, or an RNAi sequence (siRNA, shRNA, miRNA etc.), that antagonizes the function of one or more inflammatory genes, such as NF-κB, TNF-α, IL-1 (IL-1β), IL-6, Receptor activator of NF-κB (RANK), and Toll-like receptors (TLRs).

In certain embodiments, the vector of the invention encodes an antisense sequence, or an RNAi sequence (siRNA, shRNA, miRNA etc.), that antagonizes the function of a histone deacetylase, such as HDAC2. In DMD, the absence of dystrophin at the sarcolemma delocalizes and downregulates nitric oxide synthase (nNOS), which alters S-nitrosylation of HDAC2 and its chromatin association. In the dystrophin-deficient mdx mice, which are defective for the NO pathway, the activity of HDAC2 resulted to be specifically increased. In contrast, rescue of nNOS expression in mdx animals ameliorated the dystrophic phenotype. In addition, deacetylase inhibitors conferred a strong morphofunctional benefit to dystrophic muscle fibers. Indeed, givinostat, a histone deacetylase inhibitor, is under evaluation as potential disease-modifying treatment for DMD. Data indicates that, in both murine and human dystrophic cells, the absence of dystrophin correlates with HDAC2 binding to a specific subset of miRNAs (see below), while upon dystrophin rescue HDAC2 is released from these promoters.

In certain embodiments, the vector of the invention encodes an antisense sequence, an RNAi sequence (siRNA, shRNA, miRNA etc.), or a microRNA, that antagonizes the function of TGF-β, or connective tissue growth factor (CTGF). Elevated levels of TGF-β in muscular dystrophies stimulate fibrosis and impair muscle regeneration by blocking the activation of satellite cells. Anti-fibrotic agents have been tested in murine models of muscular dystrophy, including losartan, an angiotensin II-type 1 receptor blocker that reduces the expression of TGF-β. HT-100 (halofuginone) has also been shown to prevent fibrosis via the TGF-β/Smad3 pathway in muscular dystrophies. Meanwhile, FG-3019, a fully human monoclonal antibody that interferes with the action of connective tissue growth factor, a central mediator in the pathogenesis of fibrosis, has been evaluated in an open-label phase 2 trial in patients with idiopathic pulmonary fibrosis (IPF).

In certain embodiments, the vector of the invention encodes a microRNA (miR), such as miR-1, miR-29c, miR-30c, miR-133, and/or miR-206. The differential HDAC2 nitrosylation state in Duchenne versus wild-type conditions deregulates the expression of a specific subset of microRNA genes. Several circuitries controlled by the identified microRNAs, such as the one linking miR-1 to the G6PD enzyme and the redox state of cell, or miR-29 to extracellular proteins and the fibrotic process, explain some of the DMD pathogenetic traits. The muscle-specific (myomiR) miR-1 and miR-133, and the ubiquitous miR-29c and miR-30c, downregulated in mdx, recovered toward wildtype levels in exon-skipping-treated animals. According to the mdx model, when dystrophin synthesis was restored via exon skipping, the levels of miR-1, miR-133a, miR-29c, miR-30c, and miR-206 increased, while miR-23a expression did not change.

In certain embodiments, the vector of the invention encodes a microRNA inhibitor, which inhibits the function of a microRNA upregulated in DMD or its related diseases. For example, the inflammatory miR-223 expression level is upregulated in mdx mice muscles, and is downregulated in exon-skipping-treated mice. Its decrease is consistent with the observed amelioration of the inflammatory state of the muscle, due to dystrophin rescue by exon-skipping.

The mdx animals undergo extensive fibrotic degeneration, and miR-29 has been shown to target mRNAs of crucial factors involved in fibrotic degeneration, such as collagens, elastin, and structural components of the extracellular matrix. In mdx mice, miR-29 is poorly expressed, and the mRNAs for collagen (COL1A1) and elastin (ELN) were upregulated. Thus expression of miR-29c alleviates fibrotic degeneration in DMD patients, partly through downregulating collagen and elastin expression, and pathological extracellular matrix modification associated with collagen and elastin expression.

In certain embodiments, the vector of the invention encodes an antisense sequence, or an RNAi sequence (siRNA, shRNA, miRNA etc.), that antagonizes the function of G6PD (Glucose-6-phosphate dehydrogenase). One important issue in dystrophic muscles is their susceptibility and response to oxidative stress suggested to be involved in disease progression. G6PD is a cytosolic enzyme in the pentose phosphate pathway that supplies reducing energy to cells by maintaining the level of NADPH, which in turn ensures high ratio between reduced and oxidized glutathione (GSH/GSSG), GSH being the major antioxidant molecule that protects cells against oxidative damage. G6PD mRNA is deregulated in mdx muscles. It contains in its 3′-UTR region three putative binding sites for the miR-1 family, and miR-1 and miR-206 are able to repress G6PD expression. Indeed, there is an inverse correlation between G6PD and miR-1 expression: in vitro differentiation of C2 myoblasts showed that the increase in miR-1 levels correlated with decrease of G6PD protein, mRNA levels, and GSH/GSSG ratio. In mdx mice, where miR-1 is downregulated, G6PD was detected at higher levels than in WT muscles, whereas in exon-skipping-treated mdx, in which miR-1 resumes, the amount of G6PD was reduced. Notably, in mdx mice, increase in G6PD levels was accompanied by a decrease in GSH/GSSG ratio.

In certain embodiments, the vector of the invention encodes an antisense sequence, or an RNAi sequence (siRNA, shRNA, miRNA etc.), that antagonizes the function of myostatin. Myostatin is a negative regulator of muscle mass. Inhibition or blockade of endogenous myostatin compensates for the severe muscle wasting common in many types of muscular dystrophies including DMD. A myostatin blocking antibody, MYO-029, is in clinical trial for adult subjects with BMD and other dystrophies. Other clinical trials using myostatin inhibitors such as follistatin and PF-06252616 (NCT02310764) and BMS-986089 have also been conducted.

In certain embodiments, the vector of the invention encodes an antisense sequence, or an RNAi sequence (siRNA, shRNA, miRNA etc.), that antagonizes the function of phosphodiesterase-5 (PED-5) or ACE, or VEGF decoy-receptor type 1 (VEGFR-1 or Flt-1). Loss of dystrophin leads to displacement of neuronal nitric oxide synthase and reduction of muscle-derived nitric oxide to the microvasculature, resulting in functional muscle ischemia and further muscle injury. Thus several inhibitors of phosphodiesterase-5 or ACE, or VEGF decoy-receptor type 1 (VEGFR-1 or Flt-1), have been tested as part of the strategies to increase blood flow to muscles, include pharmaceutical inhibition of either phosphodiesterase-5 or ACE.

In certain embodiments, the vector of the invention encodes an antisense sequence, or an RNAi sequence (siRNA, shRNA, miRNA etc.), that antagonizes the function of hematopoietic prostaglandin D synthase (HPGDS). Prostaglandin D2 (PGD2) is produced by various inflammatory cells, and hematopoietic PGD synthase (HPGDS) is shown to be expressed in necrotic muscle of DMD patients. The administration of an HPGDS inhibitor decreased the urinary excretion of tetranor-PGDM, a urinary metabolite of PGD2, and suppressed myonecrosis in a mdx mouse model of DMD. TAS-205, a novel HPGDS inhibitor, has been evaluated for DMD treatment in clinical trial.

### RNAi and Antisense Design

In RNA interference (RNAi), short RNA molecules are created that are complimentary and bind to endogenous target mRNA. Such binding leads to functional inactivation of the target mRNA, including degradation of the target mRNA.

The RNAi pathway is found in many eukaryotes, including plants and animals, and is initiated in the cytoplasm by the enzyme Dicer, which cleaves long double-stranded RNA (dsRNA) or small hairpin RNAs (shRNA) molecules into short double-stranded fragments of ˜21 nucleotide siRNAs. Each siRNA is then unwound into two single-stranded RNAs (ssRNAs), the passenger strand and the guide strand. The passenger strand is degraded and the guide strand is incorporated into the RNA-induced silencing complex (RISC). The most well-studied outcome is post-transcriptional gene silencing, which occurs when the guide strand pairs with a complementary sequence in an mRNA molecule and induces cleavage by Argonaute 2 (Ago2), the catalytic component of the RISC. In some organisms, this process spreads systemically, despite the initially limited molar concentrations of siRNA.

Other than the siRNA and shRNA, another type of small RNA molecules that are central to RNA interference is microRNA (miRNA).

MicroRNAs are genomically encoded non-coding RNAs that help regulate gene expression, particularly during development. Mature miRNAs are structurally similar to siRNAs, but they must first undergo extensive post-transcriptional modification before reaching maturity. An miRNA is expressed from a much longer RNA-coding gene as a primary transcript known as a pri-miRNA, which is then processed in the cell nucleus to a 70-nucleotide stem-loop structure called pre-miRNA, by the microprocessor complex consisting of an RNase III enzyme Drosha and a dsRNA-binding protein DGCR8. Upon transporting this pre-miRNA into the cytosol, its dsRNA portion is bound and cleaved by Dicer to produce the mature miRNA molecule, which two strands can be separated into a passenger strand and a guide strand. The miRNA guide strand, like the siRNA guide strand, can be integrated into the same RISC complex.

Thus, the two dsRNA pathways, miRNA and siRNA/shRNA, both require processing of a precursor molecule (pri-miRNA, pre-miRNA, and dsRNA or shRNA) with a backbone sequence in order to generate the mature functional guide strand for miRNA or siRNA, and both pathways eventually converge at the RISC complex.

After integration into the RISC, siRNAs base-pair to their target mRNA and cleave it, thereby preventing it from being used as a translation template. Differently from siRNA, however, a miRNA-loaded RISC complex scans cytoplasmic mRNAs for potential complementarity. Instead of destructive cleavage (by Ago2), miRNAs target the 3′-UTR regions of mRNAs where they typically bind with imperfect complementarity, thus blocking the access of ribosomes for translation.

siRNAs differ from miRNAs in that miRNAs, especially those in animals, typically have incomplete base pairing to a target and inhibit the translation of many different mRNAs with similar sequences. In contrast, siRNAs typically base-pair perfectly and induce mRNA cleavage only in a single, specific target.

Historically, siRNA and shRNA have been used in RNAi applications. siRNA is typically a double-stranded RNA molecules, 20-25 nucleotides in length. siRNA inhibits the target mRNA transiently until they are also degraded within the cell. shRNA is typically ˜80 base pairs in length, that include a region of internal hybridization that creates a hairpin structure. As described previously, shRNA molecules are processed within the cell to form siRNA, which in turn knock down gene expression. One benefit of shRNA is that they can be incorporated into plasmid vectors and integrated into genomic DNA for longer-term or stable expression, and thus longer knockdown of the target mRNA.

shRNAs design is commercially available. For example, Cellecta offers RNAi screening service against any target gene (e.g., all 19,276 protein-encoding human genes) using the Human Genome-Wide shRNA Library or Mouse DECIPHER shRNA Library (which targets about 10,000 mouse genes). ThermoFisher Scientific provides Silencer Select siRNA (classic 21-mers) from Ambion, which, according to the manufacturer, incorporates the latest improvements in siRNA design, off-target effect prediction algorithms, and chemistry.

ThermoFisher Scientific also provides Ambion® Pre-miR™ miRNA Precursor Molecules that are small, chemically modified double-stranded RNA molecules designed to mimic endogenous mature miRNAs. Use of such Pre-miR miRNA Precursors enable miRNA functional analysis by up-regulation of miRNA activity, and can be used in miRNA target site identification and validation, screening for miRNAs that regulate the expression of a target gene, and screening for miRNAs that affect a function of the target gene (such as SLN) or a cellular process.

ThermoFisher Scientific further provides Ambion® Anti-miR™ miRNA Inhibitors, which are chemically modified, single stranded nucleic acids designed to specifically bind to and inhibit endogenous microRNA (miRNA) molecules.

Antisense sequence design is also commercially available from a number of commercial and public sources, such as IDT (Integrated DNA Technologies) and GenLink. Design considerations may include oligo length, secondary/tertiary structure in the target mRNA, protein-binding sites on target mRNA, presence of CG motifs in either the target mRNA or the antisense oligo, formation of tetraplexes in antisense oligo, and the presence of antisense activity-increasing or -decreasing motifs.

Exon skipping antisense oligo design is known in the art. See, for example, Camilla Bernardini (ed.), Duchenne Muscular Dystrophy: Methods and Protocols, Methods in Molecular Biology, vol. 1687, DOI 10.1007/978-1-4939-7374-3_10, Chapter 10 by Shimo et al., published by Springer Science+Business Media LLC, 2018), which discuss in detail the design of effective exon-skipping oligonucleotides, taking into consideration factors such as the selection of target sites, the length of the oligoes, the oligo chemistry, and the melting temperature versus the RNA strand, etc. Also discussed is the use of a cocktail of antisense oligoes to skip multiples exons. The specific genes and muscular dystrophies covered include: DMD (Duchenne muscular dystrophy), LAMA2 (merosine-deficient CMD, DYSF (dysferlinopathy, FKTN (Fukuyama CMD), DMPK (myotonic dystrophy, and SGCG (LGMD2C). The entire content is incorporated herein by reference.

For example, protein/gene sequences and mutations thereof in the affected disease genes are publically available from NCBI and the Leiden muscular dystrophy pages online. Potential target sites for efficient exon skipping can be obtained by using the human splicing finder website at www dot umd dot be slash HSF. Secondary structure of the target mRNA can be evaluated using, e.g., the mfod web server at the Albany dot edu website. The length of the oligoes normally can be 8-30 mer. Oligo GC content calculation is available at OligoCalc website at the Northwestern University server. Search for any off-target sequences can be done using the GGGenome website. Melting temperature of the oligoes can be estimated by LNA oligo prediction tool or OligoAnalyzer 3.1 software at sg dot idtdna dot com.

Enhanced Guide Strand Generation for RNAi (miR, siRNA, & shRNA)

In certain embodiments, the coding sequence encodes an RNAi reagent, such as miR, siRNA, or shRNA.

In certain embodiments, for miR and/or shRNA/siRNA design, the wild-type backbone sequence from which a mature miR or a mature siRNA is generated can be modified to enhance guide strand generation and minimize/eliminate passenger strand production. Since both strands of a mature miR/siRNA/shRNA (after cleavage) can in theory be incorporated into the RISC complex and become guide strand for RNAi, it is advantageous to selectively enhance the utilization of the designed guide strand and minimize the utilization of the largely complementary passenger strand in the RISK complex, in order to reduce or minimize, e.g., off-target effect (e.g., due to the cleavage of unintended target sequences when the passenger strand is loaded into the RISC.

One approach that can be used to achieve this goal (enhance leading strand generation and minimize/eliminate passenger strand production) is through using a hybrid construct in which the designed mature miR/siRNA/shRNA sequences comprising the desired guide strand are embedded inside the backbone sequences of other miR sequences which favor the generation of the guide strand and disfavor the production of the passenger strand.

This principle is illustrated in the design of a few modified miR-29c constructs and shSLN constructs, though the same principle that can be readily adopted for other RNAi reagents targeting any other sequences.

For all designs illustrated below, the adopted design strategy includes engineering flanking backbone sequences, loop sequences, and passenger strand nucleotide sequences, in order to preserve the 2D and 3D structure of the natural backbone sequence. In this context, for miRNA/shRNA designs, 2D/3D structure of the natural backbone sequence refers largely to the distances between stem loop and the flanking backbone polynucleotide sequences, the structure of the central stem, the location and/or sizes of the bulges, the presence and localization of any internal loops and mismatches within the stem, etc. Certain exemplary 2D structure maps for selected miR-30E, miR-101, and miR-451 backbone sequence-based miR-29c hybrid constructs are provided below as illustration.

A. Hybrid miR-29c with miR-30 Backbone Sequence (29c-M30E)

Fellmann et al. (Cell Rep. 5(6):1704-1713, 2013, incorporated herein by reference) describe a systematic approach to optimize the experimental miR-30 backbone, by identifying a conserved element 3′ of the basal stem as critically required for optimal processing of so-called “shRNAmir”—a synthetic shRNA embedded into endogenous microRNA contexts. The resulting optimized backbone, termed “miR-E,” strongly increased mature shRNA levels and knockdown efficacy. This approach can easily convert existing miR and shRNA reagents to miR-E for generating more effective miR and shRNA.

Applying this technology, 29c-M30E hybrid sequences were generated based on the desired mature miR-29c sequence, and the engineered/optimized miR-30 backbone sequence described in Fellmann et al. This 29c-M30E sequence (see FIG. 29 for its predicted 2D structure) has been incorporated into the following subject viral vectors used in the examples below: μDys-29c-M30E-i2, EF1A-29c-M30E, U6-29c-M30E. The following 5′→3′ sequence of 29c-M30E is a continuous sequence artificially separated to different lines to illustrate the different segments of the continuous sequence.

TCGACTTCTTAACCCAACAGAAGGCTCGAGAAGGTATATTGCTGTTGACA
GTGAGCGTAACCGATTTCAAATGGTGCTATAGTGAAGCCACAGATGTATA
GCACCATTTGAAATCGGTTATGCCTACTGCCTCGGACTTCAAGGGGCTAG
AATTCGA

Specifically, in the continuous sequence above, the middle line represents the passenger strand sequence, the double underlined loop sequence, and the mature miR-29c guide sequence. Note that the passenger and guide sequences can be reverse complement of each other and can snap back and form a stem-loop structure with the intervening loop sequence. However, it should be noted that perfect reverse complement sequences are not necessary. There can be internal bulges, etc., and therefore the two strands are not necessarily 100% complementary to each other in some cases (see the guide and passenger strands in the last sequence of this subsection). The top line and the bottom line represent the M30E flanking backbone sequence optimized to ensure enhanced production of the guide sequence and to minimize the production of the passenger strand.

In a similar design, an siRNA targeting human SLN is embedded in the same M30E backbone sequence in miR-30E-hSLN-c1 (compare the top and bottom rows of the sequences immediately above and below this paragraph, and the double underlined loop sequence). But the guide and passenger strands are different. This so-called c1-M30E sequence has been incorporated into the following subject viral vectors used in the examples below: c1-M30E-i2, c1-M30E-3UTR, and c1-M30E-pa.

TCGACTTCTTAACCCAACAGAAGGCTCGAGAAGGTATATTGCTGTTGACA
GTGAGCGAACTTCACTATTGTCTTGATTAC
TAGTGAAGCCACAGATGTA GTAATCAAGACAATAGTGAAGTT
TGCCTACTGCCTCGGACTTCAAGGGGCTAGAATTCGA

A similarly designed second siRNA also targeting human SLN is embedded in the same M30E backbone sequence in miR-30E-hSLN-c2 (compare the top and bottom rows of the sequences immediately above and below this paragraph, and the double underlined loop sequence). But the guide and passenger strands are different. This so-called c2-M30E sequence has been incorporated into the following subject viral vectors used in the examples below: c2-M30E-i2, c2-M30E-3UTR, and c2-M30E-pa.

TCGACTTCTTAACCCAACAGAAGGCTCGAGAAGGTATATTGCTGTTGACA
GTGAGCGAACACCCGGGAGCTGTTTCTCAA
TAGTGAAGCCACAGATGTA TTGAGAAACAGCTCCCGGGTGTT
TGCCTACTGCCTCGGACTTCAAGGGGCTAGAATTCGA

The sequence of a modified miR-29c using the natural miR-30 backbone sequence (“M30N”) is also provided below as a comparison. Note the guide strand in this case is 5′ to the loop sequence. This M30N backbone sequence similarly enhanced the production of the guide strand, though to a lesser extent than the M30E backbone sequence in the experimental system tested (data not shown).

GGTTAACCCAACAGAAGGCTAAAGAAGGTATATTGCTGTTGACAGTGAGC
GACTAGCACCATTTGAAATCGGTTA
CTGTGAAGCCACAGATGGG TAACCGATTAAATGGTGCTA
GCTGCCTACTGCCTCGGACTTCAAGGGGCTACTTTAGGA

B. Hybrid miR-29c with miR-101 Backbone Sequence (29c-101)

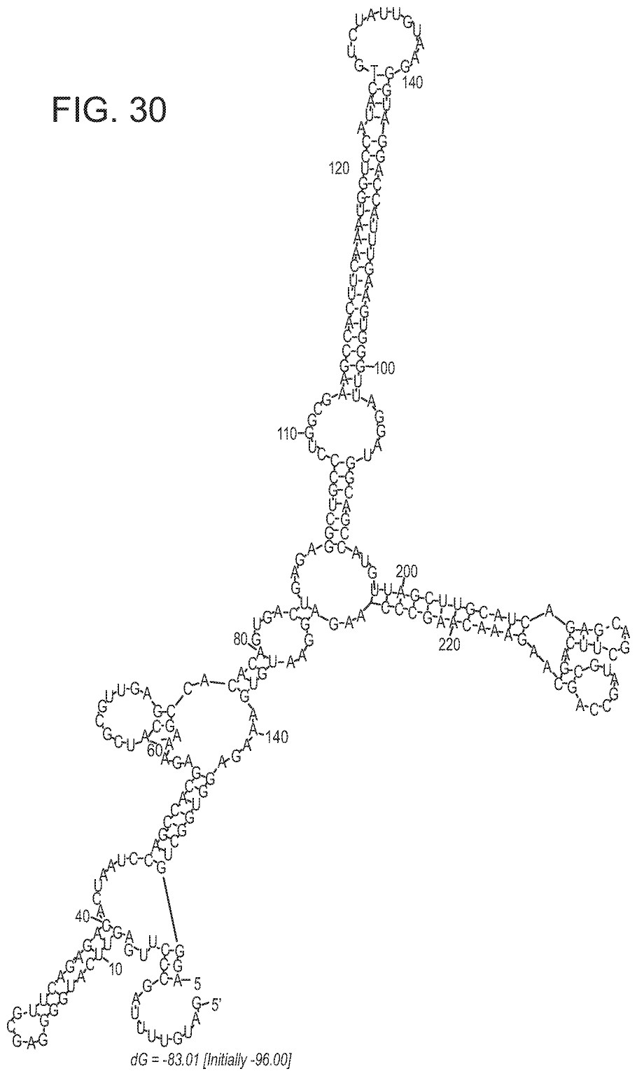

A different miR-29c hybrid (29c-101, see FIG. 30 for its predicted 2D structure) using the miR-101 backbone sequence is illustrated below using the same naming convention herein. Here, the top row and the last two rows represent the backbone sequences of miR-101, while the 2nd row is the mature miR-29c with the passenger strand, loop sequence, and guide strand. This 29c-101 sequence has been incorporated into the following subject viral vectors used in the examples below: μDys-29c-101-i2, μDys-29c-3UTR-101.

CCACCAGAAAGGATGCCGTTGACCGACACAGTGACTGACAGGCTGCCCTG
GCGAACCGATTTCAAATGGTGCATACC
GTCTATTCTAAAGG TAGCACCATTTGAAATCGGTTA
GGATGGCAGCCATCTTACCTTCCATCAGAGGAGCCTCACCGTACCCAGGA
AGAAAGAAGGTGAAAGAGGAATGTGAAACAGGTGGCTGGGA

C. Hybrid miR-29c with miR-155 Backbone Sequence (29c-155)

A different miR-29c hybrid (29c-155) using the miR-155 backbone sequence is illustrated below using the same naming convention herein. Here, the top row and the bottom row represent the flanking backbone sequences of miR-155, while the 2nd row is the mature miR-29c with the guide strand, loop sequence, and passenger strand. This 29c-155 sequence has been incorporated into the following subject viral vector used in the examples below: EF1A-29c-155.

CCTGGAGGCTTGCTGAAGGCTGTATGCTG
TAGCACCATTTGAAATCGGTTA
TTTTGGCCTCTGACTGA TGACCGCTGGAATGGTGCTA
CAGGACACAAGGCCTGTTACTAGCACTCACATGGAACAAATGGCC

Another miR-29c hybrid (29c-19nt) also using the miR-155 backbone sequence is illustrated below using the same naming convention herein. Here, the top row and the bottom row represent the flanking backbone sequences of miR-155 (identical to that in the sequence immediately above), while the 2nd row is the mature miR-29c with the guide strand, loop sequence, and passenger strand. Note the loop sequence here is 19 nt, instead of the 17 nt loop in the sequence above. This 29c-19nt sequence has been incorporated into the following subject viral vector used in the examples below: EF1A-29c-19nt, 29c-19nt-μDys-pA, 29c-19nt-μDys-3UTR.

CCTGGAGGCTTGCTGAAGGCTGTATGCTG
TAGCACCATTTGAAATCGGTTA
GTTTTGGCCACTGACTGAC TAACCGATCAAATGGTGCTA
CAGGACACAAGGCCTGTTACTAGCACTCACATGGAACAAATGGCC

D. Hybrid shSLN with miR-155 Backbone Sequence (shmSLN-v2 & c1/c2-m155)

A shSLN in the miR-155 backbone sequence is illustrated below using the same naming convention herein. Here, the top row and the bottom row represent the flanking backbone sequences of miR-155, while the 2nd row is the mature shRNA targeting mouse SLN (shmSLN) with the guide strand, loop sequence (19 nt), and passenger strand. This shmSLN-v2 sequence has been incorporated into the following subject viral vector used in the examples below: EF1A-mSLN, Fusion-v1, μDys-shmSLN-v1.

CCTGGAGGCTTGCTGAAGGCTGTATGCTG
GTGATGAGGACAACTGTGAAG
GTTTTGGCCACTGACTGAC CTTCACAGGTCCTCATCAC
CAGGACACAAGGCCTGTTACTAGCACTCACATGGAACAAATGGCC

A shSLN in the miR-155 backbone sequence is illustrated below using the same naming convention herein. Here, the top row and the bottom row represent the flanking backbone sequences of miR-155, while the 2nd row is another mature shRNA targeting mouse SLN (shmSLN) with the guide strand, loop sequence (19 nt), and passenger strand. Compared to the similar/related shmSLN sequence above, the presence of the extra dinucleotide base pairs TT:AA (or strictly speaking, UU at the 3′ end of the siRNA) have been associated with increased potency of the produced guide strand siRNA. This shmSLN-v2 sequence has been incorporated into the following subject viral vector used in the examples below: EF1A-mSLN-v2, Fusion-v2, μDys-shmSLN-v2.

CCTGGAGGCTTGCTGAAGGCTGTATGCTG
GTGATGAGGACAACTGTGAAGTT
GTTTTGGCCACTGACTGAC AACTTCACTTGTCCTCATCAC
CAGGACACAAGGCCTGTTACTAGCACTCACATGGAACAAATGGCC

Another shSLN in the miR-155 backbone sequence is illustrated below using the same naming convention herein. Here, the top row and the bottom row represent the flanking backbone sequences of miR-155, while the 2nd row is the mature shRNA targeting human SLN with the guide strand, loop sequence (19 nt), and passenger strand. This c1-m155 sequence has been incorporated into the following subject viral vector used in the examples below: c1-m155-pa, c1-m155-i2, c1-m155-3UTR.

CCTGGAGGCTTGCTGAAGGCTGTATGCTG
GTAATCAAGACAATAGTGAAGTT
GTTTTGGCCACTGACTGAC AACTTCACTTGTCTTGATTAC
CAGGACACAAGGCCTGTTACTAGCACTCACATGGAACAAATGGCC

Another shSLN in the miR-155 backbone sequence is illustrated below using the same naming convention herein. Here, the top row and the bottom row represent the flanking backbone sequences of miR-155, while the 2nd row is the mature shRNA targeting human SLN with a different guide strand, loop sequence (19 nt), and passenger strand. This c2-m155 sequence has been incorporated into the following subject viral vector used in the examples below: c2-m155-pa, c2-m155-i2, c2-m155-3UTR.

CCTGGAGGCTTGCTGAAGGCTGTATGCTG
TTGAGAAACAGCTCCCGGGTGTT
GTTTTGGCCACTGACTGAC AACACCCGGGAGCTGTTTCTCAA
CAGGACACAAGGCCTGTTACTAGCACTCACATGGAACAAATGGCC

E. Hybrid miR-29c with miR-451 Backbone Sequence (29c-451)

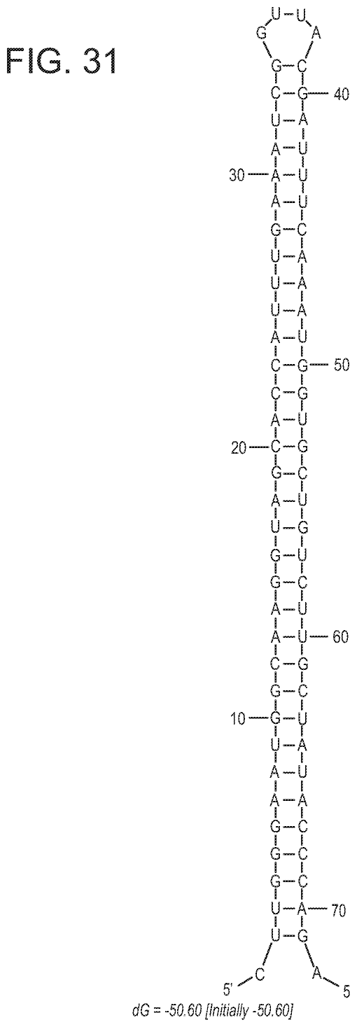

A miR-29c hybrid (29c-451, see FIG. 31 for its predicted 2D structure) using the miR-451 backbone sequence is illustrated below using the same naming convention herein. Here, the top 2 rows and the bottom 2 rows represent the flanking backbone sequences of miR-451, and the 3rd row is the mature miR-29c with the guide strand, loop sequence, and passenger strand.

GGACAGGAGAGATGCTGCAAGCCCAAGAAGCTCTCTGCTCAGCCTGTCAC
AACCTACTGACTGCCAGGGCACTTGGGAATGGCAAGG
TAGCACCATTTGAAATCGGTTA
CGATTTCAAATGGTGCTG TCTTGCTATACCCAGA
AAACGTGCCAGGAAGAGAACTCAGGACCCTGAAGCAGACTACTGGAAGGG
AGACTCCAGCTCAAACAAGGCA

F. U6 Driven miR-29c and shSLN

The experimental section below also describes the use of certain “solo” viral vector constructs that express only miR-29c or only shSLN. Such solo expression cassettes are driven by the strong Pol III U6 promoter. Such sequences do not belong to modified miR-29c or modified shSLN sequences, since the strong U6 promoter directly generates the pre-miRNA or shSLN without any flanking nucleotide sequences. For comparison purpose, however, such sequences are also listed here using the same nomenclature.

A miR-29c driven by the U6 promoter is illustrated below (U6-29c-v1). Here, the 2nd row is the mature miR-29c with the passenger strand, loop sequence, and guide strand. This has been used in the pGFP-U6-shAAV-GFP vector to generate a “solo” control vector. The nucleotides in the first row of the continuous sequence below are the first 5 nucleotides after the transcription start site in the U6 promoter, and the T₆ transcription termination sequence precedes the sequence used for cloning in the last row of the continuous sequence below.

GATCG
TAACCGATTTCAAATGGTGCTA
GCCCTGACCCAGC TAGCACCATTTGAAATCGGTTA
TTTTTTGAAGCT

A shSLN driven by the U6 promoter is illustrated below (U6-shmSLN-v1). Here, the 2nd row is the mature shSLN with the passenger strand, loop sequence, and guide strand. This has been used in the U6-shmSLN-v1 vector in the examples.

GATCG
ACTTCACAGTTGTCCTCATCAC TCGA
GTGATGAGGACAACTGTGAAG CTTTTTTGAAGCT

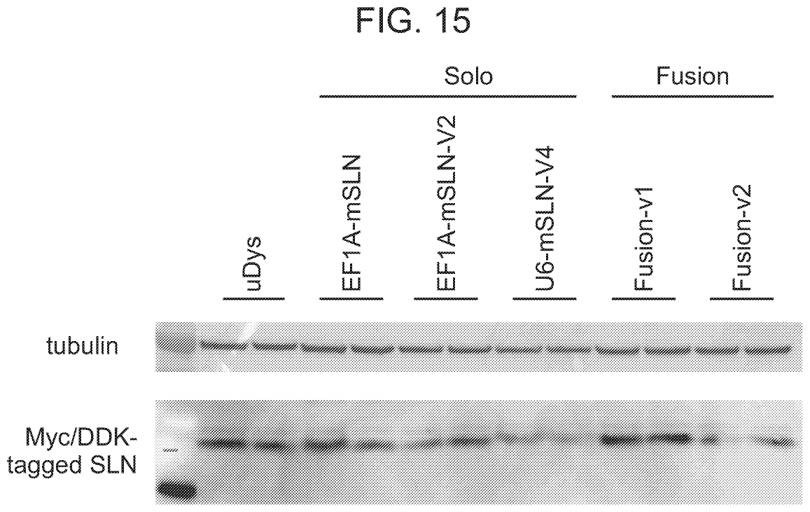

A shSLN driven by the U6 promoter is illustrated below (U6-mSLN-v4). Here, the 2nd row is the mature shSLN with the passenger strand, loop sequence, and guide strand. This has been used in the U6-mSLN-v4 vector in the examples (see FIG. 15).

GATCG
ACTTCACAGTTGTCCTCATCAC TCAAGAG
GTGATGAGGACAACTGTGAAG TTTTTTGAAGCT

### Composition and Pharmaceutical Composition

In another embodiment, the invention contemplates compositions comprising rAAV of the present invention. Compositions of the invention comprise rAAV and a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, pluronics or polyethylene glycol (PEG).

### Dosing and Administration

Titers of rAAV to be administered in methods of the invention will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³, to about 1×10¹⁴ or more DNase resistant particles (DRP) per nil. Dosages may also be expressed in units of viral genomes (vg).

Methods of transducing a target cell with rAAV, in vivo or in vitro, are contemplated by the invention. The in vivo methods comprise the step of administering an effective dose, or effective multiple doses, of a composition comprising a rAAV of the invention to an animal (including a human being) in need thereof. If the dose is administered prior to development of a disorder/disease, the administration is prophylactic. If the dose is administered after the development of a disorder/disease, the administration is therapeutic. In embodiments of the invention, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the invention is PMD or other disease characterized by defects in myelin production, degeneration, regeneration, or function.

For administration, effective amounts and therapeutically effective amounts (also referred to herein as doses) may be initially estimated based on results from in vitro assays and/or animal model studies. For example, a dose may be formulated in animal models to achieve a circulating concentration range that includes the IC₅₀ as determined in cell culture. Such information may be used to more accurately determine useful doses in subjects of interest.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the invention may be chosen and/or matched by those skilled in the art taking into account the infection and/or disease state being treated and the target cells/tissue(s) that are to express the one or more coding sequences and/or micro-dystrophin.

Specifically, the formulations described herein may be administered by, without limitation, injection, infusion, perfusion, inhalation, lavage, and/or ingestion. Routes of administration may include, but are not limited to, intravenous, intradermal, intraarterial, intraperitoneal, intralesional, intracranial, intraarticular, intraprostatic, intrapleural, intratracheal, intranasal, intravitreal, intravaginal, intrarectal, topically, intratumoral, intramuscular, intravesicular, intrapericardial, intraumbilical, intraocularal, mucosal, oral, subcutaneous, and/or subconjunctival.

The invention provides for local administration or systemic administration of an effective dose of rAAV and compositions of the invention including combination therapy of the invention. For example, systemic administration is administration into the circulatory system so that the entire body is affected. Systemic administration includes enteral administration such as absorption through the gastrointestinal tract and parental administration through injection, infusion or implantation.

In particular, actual administration of rAAV of the present invention may be accomplished by using any physical method that will transport the rAAV recombinant vector into the target tissue of an animal, such as the skeletal muscles. Administration according to the invention includes, but is not limited to, injection into muscle, the bloodstream and/or directly into the liver. Simply re-suspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein.

Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The rAAV can be used with any pharmaceutically acceptable carrier for ease of administration and handling.

The dose of rAAV to be administered in methods disclosed herein will vary depending, for example, on the particular rAAV, the mode of administration, the treatment goal, the individual, and the cell type(s) being targeted, and may be determined by methods standard in the art.

The actual dose amount administered to a particular subject may also be determined by a physician, a veterinarian, or a researcher, taking into account parameters such as, but not limited to, physical and physiological factors including body weight, severity of condition, type of disease, previous or concurrent therapeutic interventions, idiopathy of the subject, and/or route of administration.

Titers of each rAAV administered may range from about 1×10⁶, about 1×10⁷, about 1×10⁸, about 1×10⁹, about 1×10¹⁰, about 1×10¹¹, about 1×10¹², about 1×10¹³, about 1×10¹⁴, or to about 1×10¹⁵ or more DNase resistant particles (DRP) per ml. Dosages may also be expressed in units of viral genomes (vg) (i.e., 1×10⁷ vg, 1×10⁸ vg, 1×10⁹ vg, 1×10¹⁰ vg, 1×10¹¹ vg, 1×10¹² vg, 1×10¹³ vg, 1×10¹⁴ vg, 1×10¹⁵ vg, respectively). Dosages may also be expressed in units of viral genomes (vg) per kilogram (kg) of bodyweight (i.e., 1×10¹⁰ vg/kg, 1×10¹¹ vg/kg, 1×10¹² vg/kg, 1×10¹³ vg/kg, 1×10¹⁴ vg/kg, 1×10¹⁵ vg/kg respectively). Methods for titering AAV are described in Clark et al., Hum. Gene Ther. 10:1031-1039, 1999.

Exemplary doses may range from about 1×10¹⁰ to about 1×10¹⁵ vector genomes (vg)/kilogram of body weight. In some embodiments, doses may comprise 1×10¹⁰ vg/kg of body weight, 1×10¹¹ vg/kg of body weight, 1×10¹² vg/kg of body weight, 1×10¹³ vg/kg of body weight, 1×10¹⁴ vg/kg of body weight, or 1×10¹⁵ vg/kg of body weight. Doses may comprise 1×10¹⁰ vg/kg/day, 1×10¹¹ vg/kg/day, 1×10¹² vg/kg/day, 1×10¹³ vg/kg/day, 1×10¹⁴ vg/kg/day, or 1×10¹⁵ vg/kg/day. Doses may range from 0.1 mg/kg/day to 5 mg/kg/day or from 0.5 mg/kg/day to 1 mg/kg/day or from 0.1 mg/kg/day to 5 μg/kg/day or from 0.5 mg/kg/day to 1 μg/kg/day. In other non-limiting examples, a dose may comprise 1 μg/kg/day, 5 μg/kg/day, 10 μg/kg/day, 50 μg/kg/day, 100 μg/kg/day, 200 μg/kg/day, 350 μg/kg/day, 500 μg/kg/day, 1 mg/kg/day, 5 mg/kg/day, 10 mg/kg/day, 50 mg/kg/day, 100 mg/kg/day, 200 mg/kg/day, 350 mg/kg/day, 500 mg/kg/day, or 1000 mg/kg/day. Therapeutically effective amounts may be achieved by administering single or multiple doses during the course of a treatment regimen (i.e., days, weeks, months, etc.).

In some embodiments, the pharmaceutical composition is in a dosage form of 10 mL of aqueous solution having at least 1.6×10¹³ vector genomes. In some embodiments, the dosage has a potency of at least 2×10¹² vector genomes per milliliter. In some embodiments, the dosage comprises a sterile aqueous solution comprising 10 mM L-histidine at pH 6.0, 150 mM sodium chloride, and 1 mM magnesium chloride. In some embodiments, the pharmaceutical composition is in a dosage form of 10 mL of a sterile aqueous solution comprising 10 mM L-histidine at pH 6.0, 150 mM sodium chloride, and 1 mM magnesium chloride; and having at least 1.6×10¹³ vector genomes.

In some embodiments, the pharmaceutical composition may be a dosage comprising between 1×10¹⁰ and 1×10¹⁵ vector genomes in 10 mL aqueous solution; between 1×10¹¹ and 1×10¹⁴ vector genomes in 10 mL aqueous solution; between 1×10¹² and 2×10¹³ vector genomes in 10 mL aqueous solution; or greater than or equal to about 1.6×10¹³ vector genomes in 10 mL aqueous solution. In some embodiments the aqueous solution is a sterile aqueous solution comprises about 10 mM L-histidine pH 6.0, with 150 mM sodium chloride, and 1 mM magnesium chloride. In some embodiments, the dosage has a potency of greater than about 1×10¹¹ vector genomes per milliliter (vg/mL), greater than about 1×10¹² vg/mL, greater than about 2×10¹² vg/mL, greater than about 3×10¹² vg/mL, or greater than about 4×10¹² vg/mL.

In some embodiments, at least one AAV vector is provided as part of a pharmaceutical composition. The pharmaceutical composition may comprise, for example, at least 0.1% w/v of the AAV vector. In some other embodiments, the pharmaceutical composition may comprise between 2% to 75% of compound per weight of the pharmaceutical composition, or between 25% to 60% of compound per weight of the pharmaceutical composition.

In some embodiments, the dosage is in a kit. The kit may further include directions for use of the dosage.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

In some embodiments, for injection, formulations may be made as aqueous solutions, such as in buffers including, but not limited to, Hanks' solution, Ringer's solution, and/or physiological saline. The solutions may contain formulatory agents such as suspending, stabilizing, and/or dispersing agents. Alternatively, the formulation may be in lyophilized and/or powder form for constitution with a suitable vehicle control (e.g., sterile pyrogen-free water) before use.

Any formulation disclosed herein may advantageously comprise any other pharmaceutically acceptable carrier or carriers which comprise those that do not produce significantly adverse, allergic, or other untoward reactions that may outweigh the benefit of administration, whether for research, prophylactic, and/or therapeutic treatments. Exemplary pharmaceutically acceptable carriers and formulations are disclosed in Remington's Pharmaceutical Sciences, 18th Ed., Mack Printing Company, 1990, which is incorporated by reference herein for its teachings regarding the same. Moreover, formulations may be prepared to meet sterility, pyrogenicity, general safety, and purity standards as required by the United States FDA's Division of Biological Standards and Quality Control and/or other relevant U.S. and foreign regulatory agencies.

Exemplary, generally used pharmaceutically acceptable carriers may comprise, but are not limited to, bulking agents or fillers, solvents or co-solvents, dispersion media, coatings, surfactants, antioxidants (e.g., ascorbic acid, methionine, and vitamin E), preservatives, isotonic agents, absorption delaying agents, salts, stabilizers, buffering agents, chelating agents (e.g., EDTA), gels, binders, disintegration agents, and/or lubricants.

Exemplary buffering agents may comprise, but are not limited to, citrate buffers, succinate buffers, tartrate buffers, fumarate buffers, gluconate buffers, oxalate buffers, lactate buffers, acetate buffers, phosphate buffers, histidine buffers, and/or trimethylamine salts.

Exemplary preservatives may comprise, but are not limited to, phenol, benzyl alcohol, meta-cresol, methylparaben, propyl paraben, octadecyldimethylbenzyl ammonium chloride, benzalkonium halides, hexamethonium chloride, alkyl parabens (such as methyl or propyl paraben), catechol, resorcinol, cyclohexanol, and/or 3-pentanol.

Exemplary isotonic agents may comprise polyhydric sugar alcohols comprising, but not limited to, trihydric or higher sugar alcohols, (e.g., glycerin, erythritol, arabitol, xylitol, sorbitol, and/or mannitol).

Exemplary stabilizers may comprise, but are not limited to, organic sugars, polyhydric sugar alcohols, polyethylene glycol, sulfur-containing reducing agents, amino acids, low molecular weight polypeptides, proteins, immunoglobulins, hydrophilic polymers, and/or polysaccharides.

Formulations may also be depot preparations. In some embodiments, such long-acting formulations may be administered by, without limitation, implantation (e.g., subcutaneously or intramuscularly) or by intramuscular injection. Thus, for example, compounds may be formulated with suitable polymeric and/or hydrophobic materials (e.g., as an emulsion in an acceptable oil) or ion exchange resins, or as sparingly soluble derivatives (e.g., as a sparingly soluble salt).

Additionally, in various embodiments, the AAV vectors may be delivered using sustained-release systems, such as semipermeable matrices of solid polymers comprising the AAV vector. Various sustained-release materials have been established and are well known by those of ordinary skill in the art. Sustained-release capsules may, depending on their chemical nature, release the vector following administration for a few weeks up to over 100 days.

The pharmaceutical carriers, diluents or excipients suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the invention results in sustained co-expression of said one or more additional coding sequences and micro-dystrophin. The present invention thus provides methods of administering/delivering rAAV which co-expresses said one or more additional coding sequences and micro-dystrophin to an animal, preferably a human being. These methods include transducing tissues (including, but not limited to, tissues such as muscle, organs such as liver and brain, and glands such as salivary glands) with one or more rAAV of the present invention. Transduction may be carried out with gene cassettes comprising tissue specific control elements. For example, one embodiment of the invention provides methods of transducing muscle cells and muscle tissues directed by muscle specific control elements, including, but not limited to, those derived from the actin and myosin gene families, such as from the myoD gene family (See Weintraub et al., Science 251:761-766, 1991), the myocyte-specific enhancer binding factor MEF-2 (Cserjesi and Olson, Mol Cell Biol 11:4854-4862, 1991), control elements derived from the human skeletal actin gene (Muscat et al., Mol Cell Biol 7:4089-4099, 1987), the cardiac actin gene, muscle creatine kinase sequence elements (Johnson et al., Mol Cell Biol 9:3393-3399, 1989), and the murine creatine kinase enhancer (mCK) element, control elements derived from the skeletal fast-twitch troponin C gene, slow-twitch cardiac troponin C gene and the slow-twitch troponin I gene: hypoxia-inducible nuclear factors (Semenza et al., Proc Natl Acad Sci U.S.A. 88:5680-5684, 1991), steroid-inducible elements and promoters including the glucocorticoid response element (GRE) (See Mader and White, Proc. Natl. Acad. Sci. U.S.A. 90:5603-5607, 1993), and other control elements.

Muscle tissue is an attractive target for in vivo DNA delivery, because it is not a vital organ and is easy to access. The invention contemplates sustained co-expression of miRNAs and micro-dystrophin from transduced myofibers.

As used herein, “muscle cell” or “muscle tissue” is meant a cell or group of cells derived from muscle of any kind (for example, skeletal muscle and smooth muscle, e.g., from the digestive tract, urinary bladder, blood vessels or cardiac tissue). Such muscle cells may be differentiated or undifferentiated, such as myoblasts, myocytes, myotubes, cardiomyocytes and cardiomyoblasts.

The term “transduction” is used to refer to the administration/delivery of the one or more additional coding sequences and the coding region of the micro-dystrophin to a recipient cell either in vivo or in vitro, via a replication-deficient rAAV of the invention resulting in co-expression of the one or more additional coding sequences and micro-dystrophin by the recipient cell.

Thus, the invention provides methods of administering an effective dose (or doses, administered essentially simultaneously or doses given at intervals) of rAAV that encode said one or more additional coding sequences and micro-dystrophin to a patient in need thereof.

### AAV Production

Genes encoding the necessary replication (rep) and structural (cap) proteins of AAV vectors have been deleted from AAV vectors to allow insertion of the sequences to be delivered between the remaining terminal repeat sequences. Thus for growth of AAV vectors, not only is a helper virus required, but the genes encoding the rep and cap proteins need to be delivered to infected cells. Alternatively, the genes encoding the rep and cap proteins need to be present in the cells used for production.

AAV vectors suitable for the methods of the invention can be produced using any of the art-recognized methods. In a recent review, Penaud-Budloo et al. (Molecular Therapy: Methods & Clinical Development Vol. 8, pages 166-180, 2018) provided a review of the most commonly used upstream methods to produce rAAVs. Each methods described therein are incorporated herein by reference.

### Transient Transfection of Packaging Cell Line (HEK293)

In particular, in certain embodiments, the AAV vector is produced using transient transfection of a packaging cell line such as HEK293 cells. This is the most established AAV production method comprising plasmid transfection of human embryonic HEK293 cells. Typically, HEK293 cells are simultaneously transfected by a vector plasmid (containing the gene of interest, such as the subject polynucleotide encoding both the dystrophin minigene and the one or more additional coding sequences), and one or two helper plasmids, using calcium phosphate or polyethylenimine (PEI), a cationic polymer.

The helper plasmid(s) allow the expression of the four Rep proteins, the three AAV structural proteins VP1, VP2, and VP3, the AAP, and the adenoviral auxiliary functions E2A, E4, and VARNA. The additional adenoviral E1A/E1B co-factors necessary for rAAV replication are ex-pressed in HEK293 producer cells. Rep-cap and adenoviral helper sequences are either cloned on two separate plasmids or combined on one plasmid, hence both a triple plasmid system and a two plasmid system for transfection are possible. The triple plasmid protocol lends versatility with a cap gene that can easily be switched from one serotype to another.

The plasmids are usually produced by conventional techniques in E. coli using bacterial origin and anti-biotic-resistance gene or by minicircle technology.

Transient transfection in adherent HEK293 cells has been used for large-scale manufacturing of rAAV vectors. Recently, HEK293 cells have also been adapted to suspension conditions to be economically viable in the long term.

HEK293 lines are usually propagated in DMEM completed with L-glutamine, 5%-10% of fetal bovine serum (FBS), and 1% penicillin-streptomycin, except for suspension HEK293 cells that are maintained in serum-free suspension F17, Expi293, or other manufacturer-specific media. For adherent cells, the percentage of FBS can be reduced during AAV production in order to limit contamination by animal-derived components.

Generally, the rAAV vectors are recovered 48-72 hr after plasmid transfection from the cell pellet and/or supernatant, depending on the serotype.

Infection of Insect Cells with Recombinant Baculovirus

The baculovirus-Sf9 platform has been established as a GMP-compatible and scalable alternative AAV production method in mammalian cells. It can generate up to 2×10⁵ vector genomes (vg) per cell in crude harvests.

Current protocol involves infection of the Sf9 insect cells with two recombinant baculoviruses a baculovirus expression vector (BEV) allowing the synthesis of Rep78/52 and Caps, and a recombinant baculovirus carrying the gene of interest flanked by the AAV ITRs. Several serum-free media are adapted for Sf9 cell growth in suspension.

The dual-baculovirus-Sf9 production system has many advantages over other production platforms regarding these safety issues: (1) the use of serum-free media; (2) despite the discovery of adventitious virus transcripts in Sf cell lines, most of the viruses infecting insects do not replicate actively in mammalian cells; and (3) no helper virus is required for rAAV production in insect cells besides baculovirus.

In certain embodiments, stable Sf9 insect cell lines expressing Rep and Cap proteins are used, thus requiring the infection of only one recombinant baculovirus for the production of infectious rAAV vectors at high yield.

Infection of Mammalian Cells with rHSV Vectors

HSV is a helper virus for replication of AAV in permissive cells. Thus, the HSV can serve both as a helper and as a shuttle to deliver the necessary AAV functions that support AAV genome replication and packaging to the producing cells.

AAV production based on co-infection with rHSV can efficiently generate a large amount of rAAV. In addition to high overall yields (up to 1.5×10⁵ vg/cell), the method is further advantageous in that it creates rAAV stocks with apparently increased quality as measured by an improved viral potency.

In this method, cells, typically the hamster BHK21 cell line or the HEK293 and derivatives, are infected with two rHSVs, one carrying the gene of interest bracketed by AAV ITR (rHSV-AAV), and the second with the AAV rep and cap ORFs of the desired serotype (rHSVrepcap). After 2-3 days, the cells and/or the media are collected, and rAAV is purified over multiple purification steps to remove cellular impurities, HSV-derived contaminants, and unpackaged AAV DNA.

Thus in some embodiments, HSV serves as a helper virus for AAV infection. In some embodiments, AAV growth is accomplished using non-replicating mutants of HSV with ICP27 deleted.

Certain methods for producing recombinant AAV viral particles in a mammalian cell have been known in the art and improved over the past decade. For example, U.S. Application Publication No. 20070202587 describes recombinant AAV production in mammalian cells based on co-infection of the cells with two or more replication-defective recombinant HSV vectors. U.S. Application Publication No. 20110229971 and Thomas et al. (Hum. Gene Ther. 20(8):861-870, 2009) describes a scalable recombinant AAV production method using recombinant HSV type 1 coinfection of suspension-adapted mammalian cells. Adamson-Small et al. (Hum. Gene Ther. Methods 28(1):1-14, 2017) describes an improved AAV production method in a serum-free suspension manufacturing platform using the HSV system.

### Mammalian Stable Cell Lines

rAAV vectors can also be efficiently and scalably produced using stable mammalian producer cells stably expressing rep and cap genes. Such cells can be infected by wild-type Ad5 helper virus (which is genetically stable and can be easily produced at high titers) to induce high-level expression of rep and cap. Infectious rAAV vectors can be generated upon infection of these packaging cells lines with wild-type Ad type 5, and providing the rAAV genome by either plasmid transfection or after infection with a recombinant Ad/AAV hybrid virus.

Alternatively, Ad can be replaced by HSV-1 as the helper virus.

Suitable stable mammalian producer cells may include HeLa-derived producer cell lines, A549 cells, or HEK293 cells. A preferred HeLa cell line is HeLaS3 cells, a suspension adapted HeLa subclone.

The methods herein described can be used to manufacture the subject AAV vectors in animal components-free medium, preferably at 250-L scale, or 2,000-L commercial scale.

### EXAMPLES

### Example 1 Expression Validation of microD5 (SGT001) Constructs in C2C12 Cells

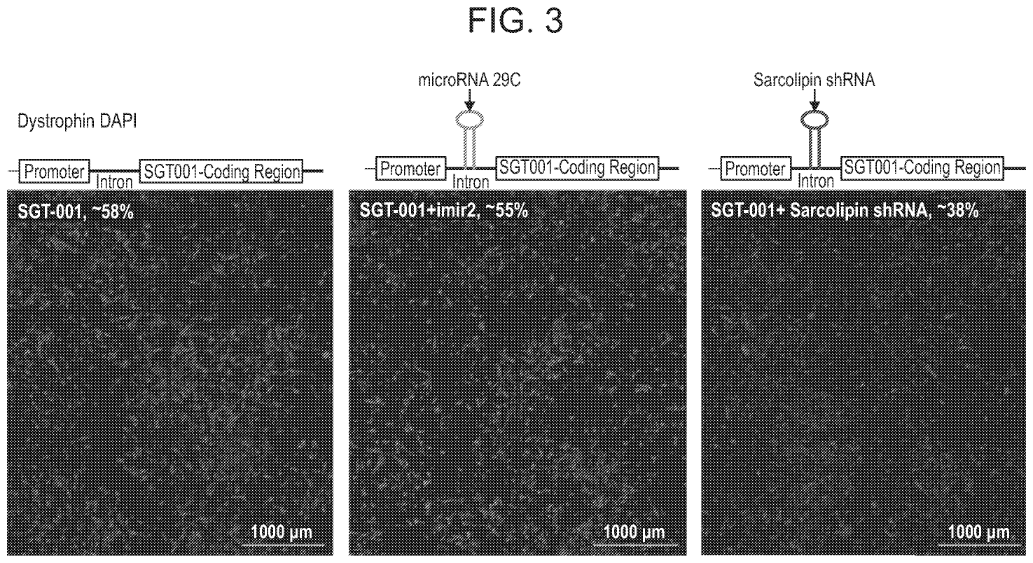

To confirm that the microD5 (SGT-001) micro-dystrophin transgene can be expressed in vitro with or without additional coding sequences for microRNA (such as miR-29c coding sequence) or shRNA (such as shRNA against SLN) inserted into the same AAV vector, C2C12 cells were infected in vitro by three AAV viral constructs: one encoding the wild-type microD5 (SGT-001) construct; one encoding a fusion construct of microD5 (SGT-001) and miR-29c, wherein the miR-29c coding sequence was inserted into the heterologous intron region 5′ to the microD5 (SGT-001) coding sequence; and one encoding a fusion construct of microD5 (SGT-001) and shRNA against SLN, wherein the coding sequence for shRNA was inserted into the heterologous intron region 5′ to the SGT-001 coding sequence. See FIG. 3.

It was predicted that the microD5 (SGT-001)/miR-29c fusion construct would produce an initial transcript encoding both the dystrophin minigene product and the miR-29c RNA. It was also predicted that the microD5 (SGT-001)/shRNA against SLN fusion construct would produce an initial transcript encoding both the dystrophin minigene product and the shRNA.

While not wishing to be bound by any particular theory, Applicant also believed that subsequent processing of the fusion transcripts may result in pre-mature cleavage of the fusion mRNA (such as causing the loss of the polyA tail). This may have contributed to the reduced expression of microdystrophin in C2C12 cells infected by the fusion constructs, compared to that infected by the wild-type microdystrophin construction without the additional coding sequence. See FIG. 3.

However, the experiments did confirm that the microD5 (SGT-001) construct successfully expressed the desired microdystrophin protein (as evidence by immunofluorescent staining using antibodies specific for the microdystrophin gene product), and the expression persists in C2C12 cells infected with the miR-29c or the shRNA fusion constructs, although at a slightly reduced/inhibited/suppressed level.

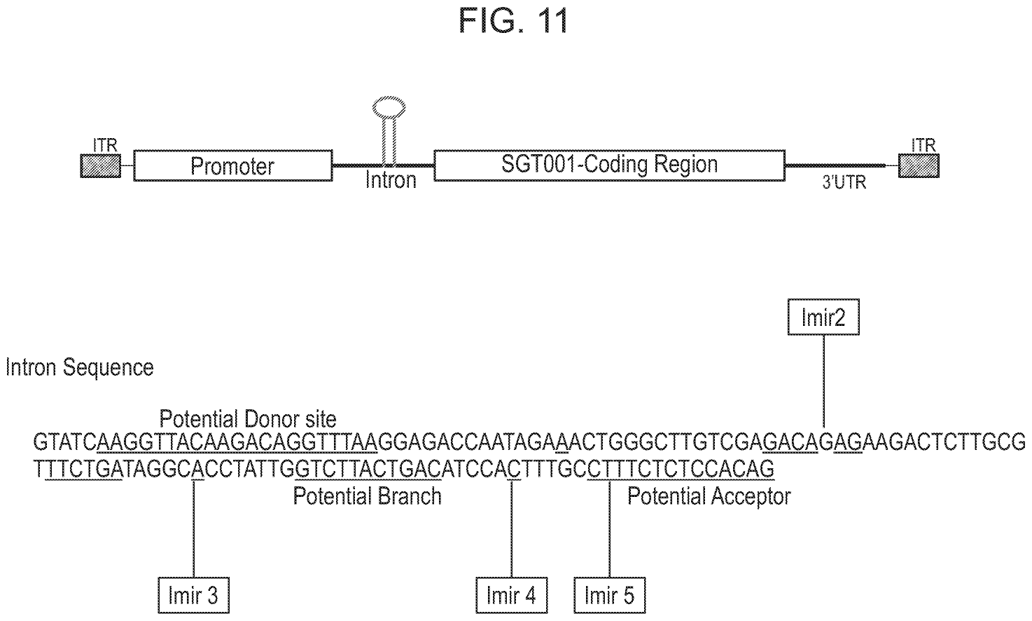

Similar experiments were also conducted for other fusion constructs, in which the miR-29c coding sequence was inserted into different locations in the heterologous intron between the promoter and the microD5 (SGT-001) coding sequence. See FIG. 11 for the insertion locations for the various fusion constructs Imir2, Imir3, Imir4, and Imir5.

Insertion was confirmed after amplifying the intron sequence with or without the inserted coding sequence by PCR, and the amplification product analyzed by electrophoresis. For example, the insertion of the 88-bp miR-29c coding sequence increased the size of the PCT amplification product by just under 100 bp.

The fusion constructs were then used in several experiments to determine whether microD5 (SGT-001) expression in infected C2C12 cells was affected.

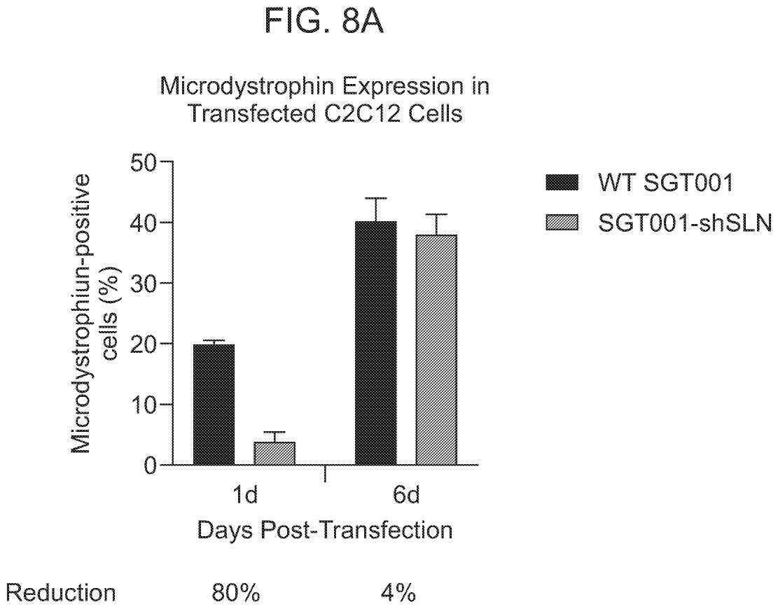

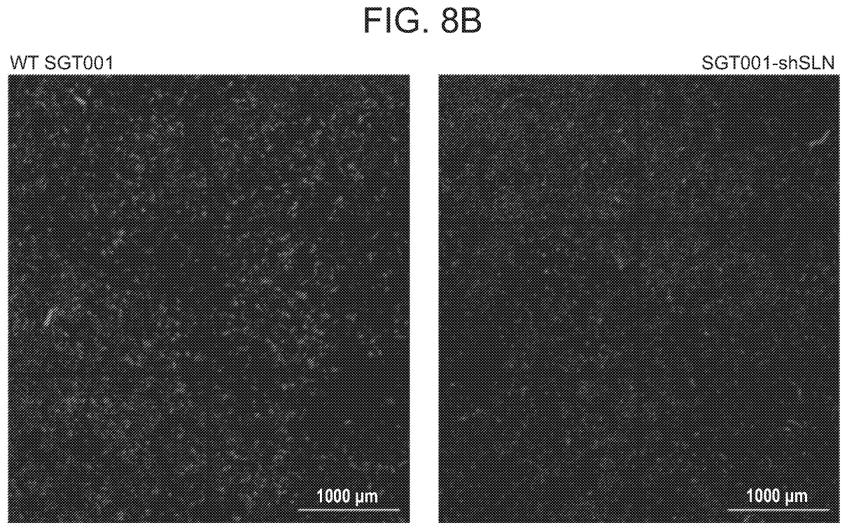

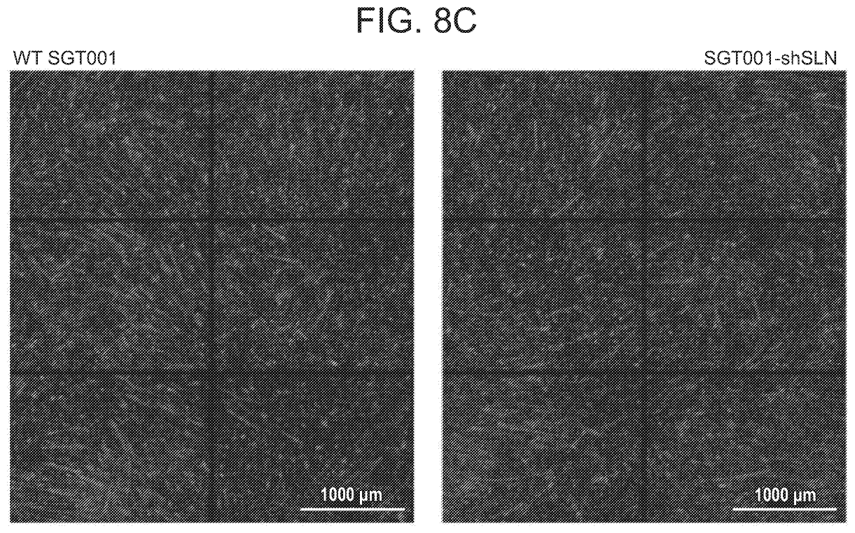

It is apparent that in this particular experiment, the presence of the coding sequence for shRNA initially impeded microdystrophin expression in transfected C2C12 cells, one day post transfection. However, microdystrophin expression quickly caught up, such that by day 6 post transfection, microdystrophin expression in transfected C2C12 cells was virtually the same, with or without the additional coding sequence for shRNA on the AAV vector. See FIGS. 8A-8C.

The data demonstrates that the subject AAV constructs can encompass additional coding sequences for shRNA or microRNA without significantly affect microdystrophin gene expression.

### Example 2 Functional Assay for shRNA Against Sarcolipin (SLN)

This example demonstrates that the coding sequence for shRNA against SLN (shSLN) inserted into the subject AAV vector can produce a functional shRNA that reduces expression of SLN.

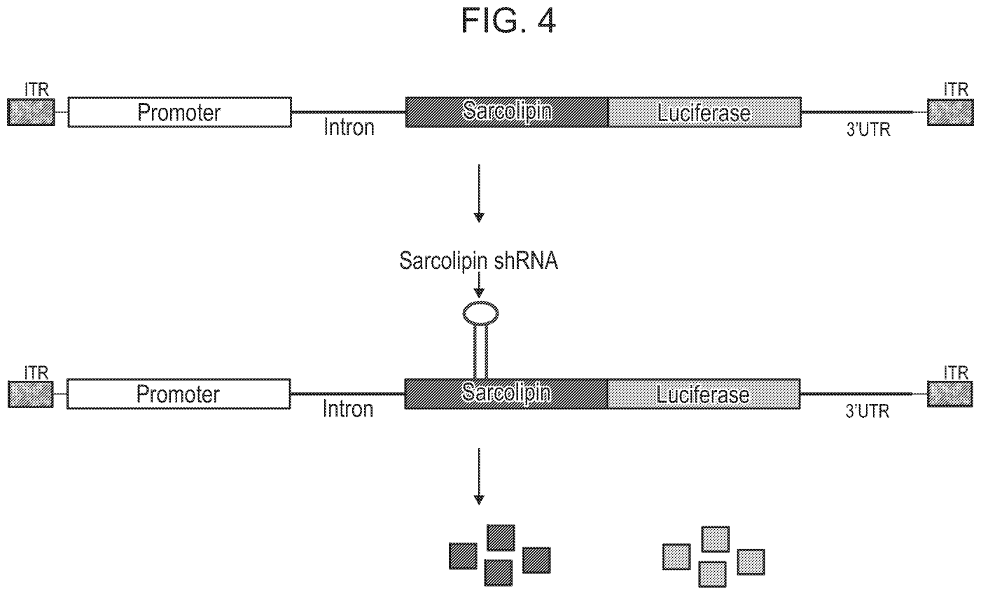

FIG. 4 is a schematic drawing showing a sarcolipin-luciferase fusion reporter encoded by an AAV vector. Upon co-transfecting the luciferase-bearing reporter, and an AAV encoding a microD5 with or without shSLN, into the C2C12 cells, the ability of the encoded shSLN to reduce the expression of the SLN-luciferase fusion can be assessed based on the luciferase generated signal.

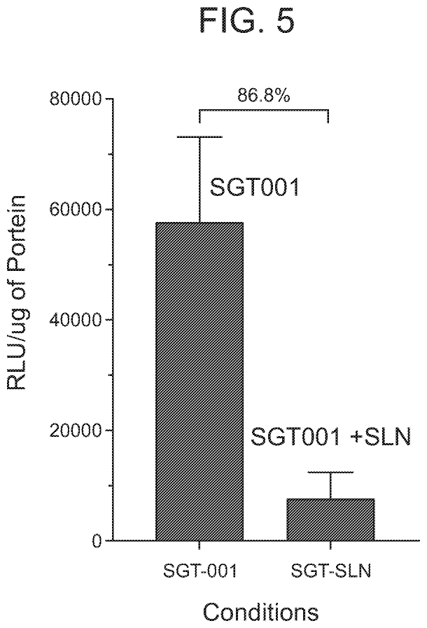

FIG. 5 shows a representative result of such a co-transfection experiment. Specifically, in one experiment, co-transfection of the SLN-luciferase fusion reporter construct with an AAV vector encoding microD5 only (“SGT001”) resulted in strong luciferase signal. Meanwhile, in another experiment, co-transfection of the SLN-luciferase fusion reporter construct with an AAV vector encoding microD5 and shSLN (“SGT001+SLN”) resulted in 86.7% reduction of the luciferase signal, suggesting that the shSLN was expressed and was effective to reduce target gene (the microD5-luciferase fusion) expression.

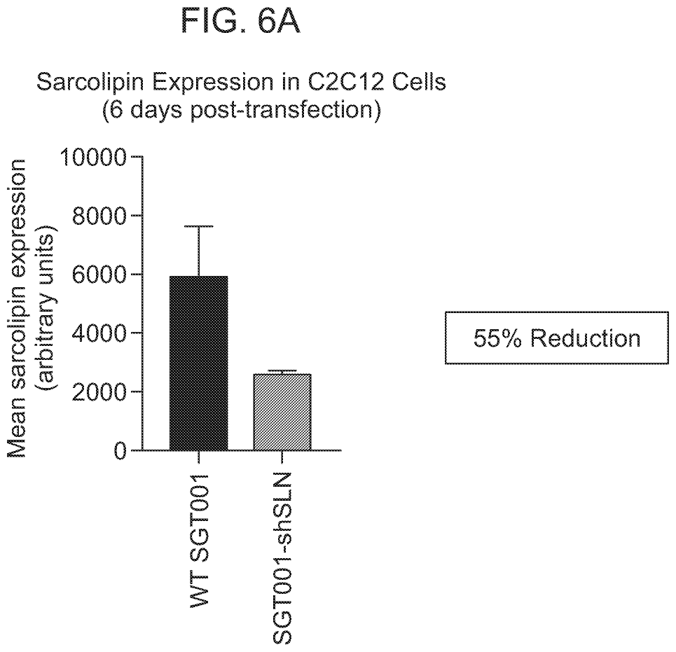

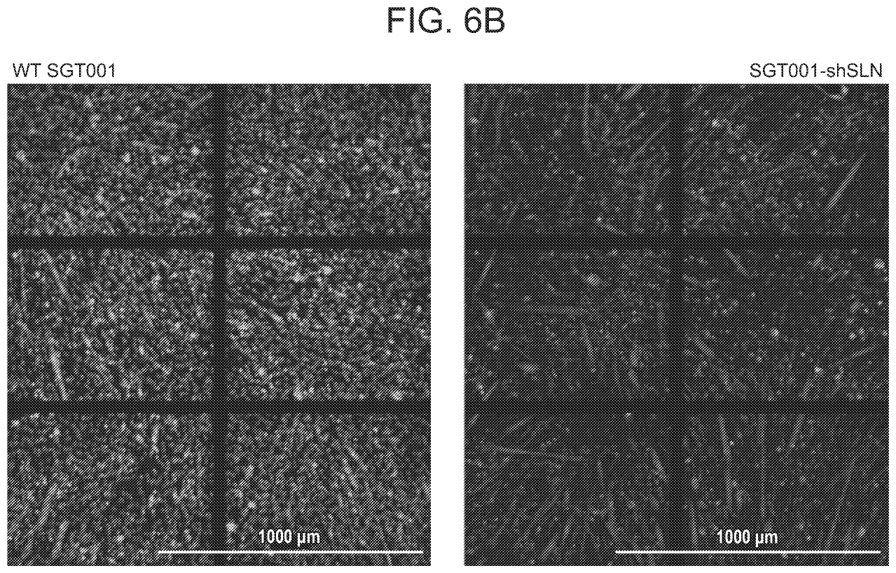

FIG. 6A shows the results of the shRNA(SLN) functional test based on direct immunostaining of sarcolipin. In C2C12 cells transfected with AAV9 vector encoding microD5 (SGT-001) dystrophin minigene and the coding sequence for shRNA (shSLN), endogenous SLN expression was reduced by 55%, compared to C2C12 cells transfected with the same vector encoding only the microD5 (SGT-001) dystrophin minigene. See immunofluorescent image in FIG. 6B.

The function of the shRNA(SLN) coding sequence in the AAV vector was further tested based on measuring calcium re-uptake into sarcoplasmic reticulum.

Sarco(Endo)plasmic Reticulum Ca²⁺-ATPases (SERCAs) are transmembrane proteins that catalyze the ATP-dependent transport of Ca²⁺ from the cytosol into the lumen of the sarcoplasmic reticulum in muscle cells. Sarcolipin encoded by the SLN gene is a small transmembrane proteolipid that regulates several SERCAs by reducing the accumulation of Ca²⁺ in the sarcoplasmic reticulum without affecting the rate of ATP hydrolysis. Ablation of sarcolipin increases atrial Ca²⁺ transient amplitudes and enhanced atrial contractility. Furthermore, atria from sarcolipin-null mice have blunted response to isoproterenol stimulation, implicating sarcolipin as a mediator of beta-adrenergic responses in atria.

Thus a functional shRNA(SLN) would be expected to reduce the expression level of endogenous SLN, leading to less SLN binding to SERCA and thus less impediment of calcium re-uptake into the sarcoplasmic reticulum. Phenotypically, the effect of expressing a functional shRNA(SLN) would be similar to overexpressing SERCA, such as SERCA2a, the overexpression of which in rats has been shown to reduce the relaxation time for right ventricular papillary muscles, suggesting faster calcium reuptake into the sarcoplasmic reticulum.

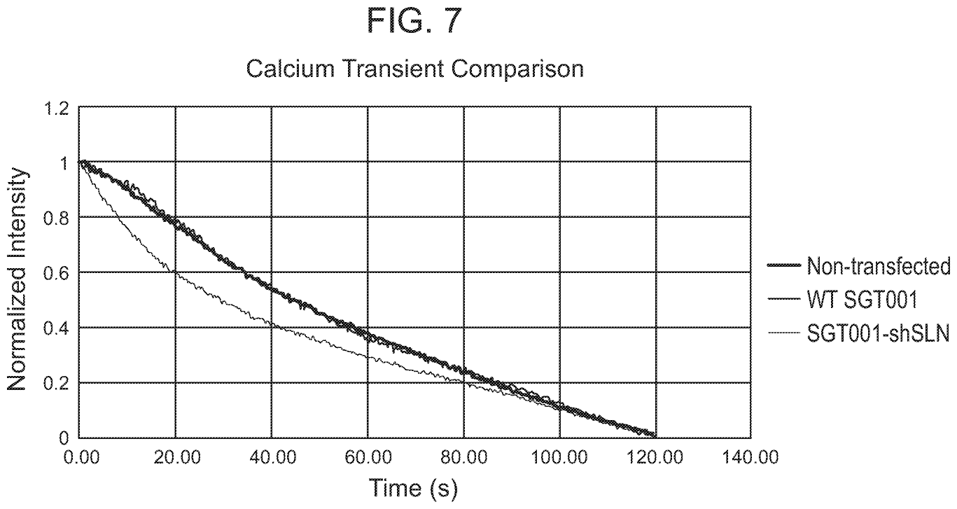

Indeed, faster calcium reuptake into the sarcoplasmic reticulum is what is shown in FIG. 7, which shows normalized fluorescent signals emitted by the calcium-binding dye Fluo-8.

Fluo-8 (Abcam) is a cell-permeable medium affinity green fluorescent calcium binding dye. It binds to intracellular calcium at a K_{d} of about 390 nM. Its fluorescence intensity increases upon Ca²⁺ binding.

A faster reduction in Fluo-8 signal represents a faster reduction of cytosolic calcium in C2C12 cells infected by AAV vector encoding microD5 (SGT-001)-shSLN, compared to non-transfected C2C12 cells, and C2C12 cells infected by AAV vector encoding the microD5 (SGT-001) dystrophin minigene only.

On the other hand, microD5 (SGT-001) dystrophin minigene expression remains largely unaffected by the presence of the shSLN coding sequence or shSLN expression, several days (e.g., 6 days) after the cells were infected by the AAV construct, although microD5 (SGT-001) dystrophin minigene expression was initially reduced (i.e., 1 day post infection). See FIGS. 8A-8C.

In comparison, at Day 6 post infection, endogenous SLN expression is reduced by 55%. See FIG. 6A. This is consistent with the 86% reduction seen in the SLN-Luciferase reporter assay in FIG. 5.

These data supports the notion that the subject AAV vector co-expressing both a micro-dystrophin and shSLN can efficiently express both transgenes on the same AAV vector, while expression of shSLN does not negatively impact microdystrophin minigene expression in the long term.

Furthermore, the expressed shSLN appears to be functional, based both on the luciferase reporter assay, as well as direct measurement of endogenous SLN expression.

### Example 3 In Vitro Expression of Coding Sequences from Fusion Constructs

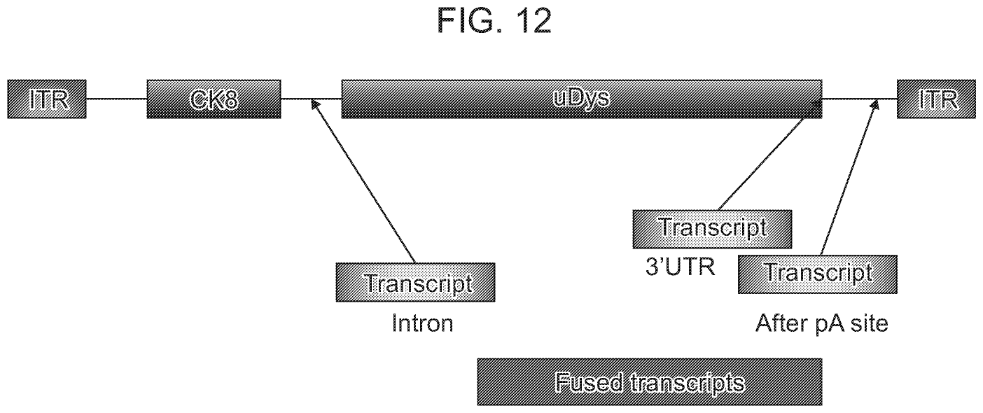

The fusion viral vectors of the invention are capable of expressing not only the functional gene or protein of interest (GOI) but also one or more coding sequences for certain RNAi, antisense, sgRNA, miRNA or inhibitors thereof. A representative, non-limiting configuration of the recombinant viral vector of the invention is illustrated in FIG. 12. For example, the recombinant viral vector of the invention may be a fusion AAV vector, such as AAV9 vector, designed to express a version of a functional dystrophin gene such as any one of the μDys gene described herein above. The same fusion vector also expresses one or more additional coding sequence(s) within the intron, within 3′-UTR, or after the polyA signal in the initial transcript, such as after the polyA signal but before the transcription termination sequence, or after the transcription termination sequence, of the recombinant AAV9 vector.

In case that the additional coding sequence encodes an miRNA, such as miR-29c, the backbone sequence of the miR-29c coding sequence can be modified such that the surrounding sequences for the mature miR-29c sequence are obtained from other miRNA, such as that for miR-30, -101, -155, or -451 (see above). It has been found that replacing the native surrounding sequences of miR-29c by those from miR-30, -101, -155, or -451 can enhance the production of the one strand (i.e., the guide strand) of miR-29c designed to target the miR-29c target sequence (i.e., reduce the production of its complement passenger strand that is not useful for targeting the miR-29c target sequence).

As controls, several so-called solo expression constructs were generated on the same vector background. These solo expressing constructs do not express μDys gene, but may instead express a reporter gene such as EGFP or GFP.

For example, one such solo vector may express the miR-29c coding sequence inserted into the intron sequence upstream of an EGFP coding sequence, all from an EF1A promoter. The backbone sequences of the miR-29c coding sequence may be modified by that of miR-30, -101, -155, or -451.

Another such solo vector may express an shRNA, such as shSLN that targets/down-regulates the expression of SLN. Expression of the shRNA may be driven by the U6 promoter that can be used by RNA Pol III, which produces strong transcription of short RNA transcripts. The shRNA coding sequence can be inserted into the intron in the U6 transcription cassette, before a coding sequence for GFP.

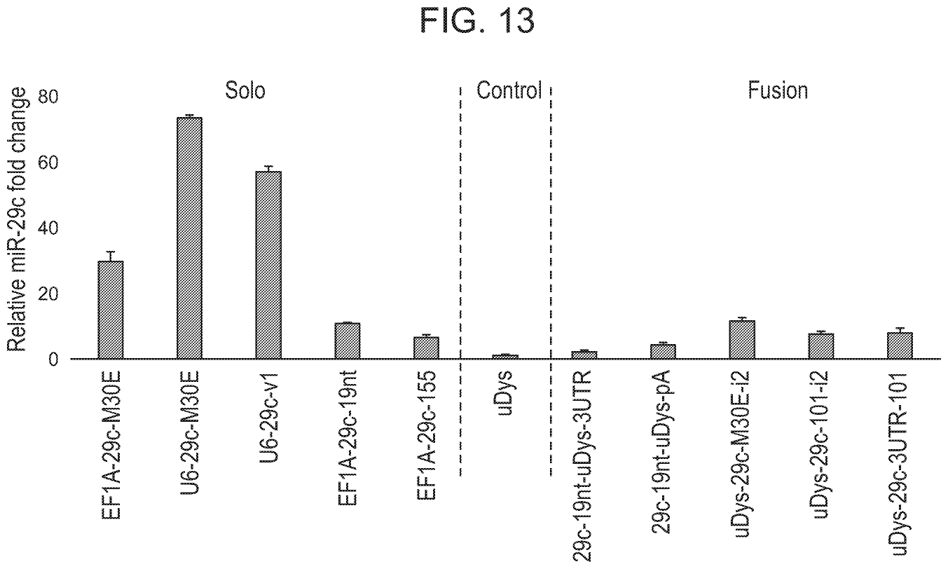

Several such representative fusion or solo vectors were used to transfect human iPS-derived cardiomyocytes in vitro, and the expression of miR-29c in the infected cardiomyocytes were determined, and the results were shown in FIG. 13.

Specifically, the five solo constructs, five fusion constructs, and a control μDys expressing construct were transfected to human iPS-derived cardiomyocytes according to standard procedure. Mature miR-29c levels were measured via Taqman stem-loop QPCR. The five solo constructs tested include U6- or EF1A-driven miR-29c expression cassettes designed in miR-30 (EF1A-29c-M30E and U6-29c-M30E) and miR-155 (EF1A-29c-19nt and EF1A-29c-155) backbones. The five fusion constructs tested include miR-29c expression cassettes designed in miR-101 (μDys-29c-10142 & μDys-29c-3UTR-101), miR-30 (μDys-29c-M30E-i2), and miR-155 (29c-19nt-μDys-3UTR & 29c-19nt-μDys-pa) backbones, inserted into intronic (i2), 3′UTR (3UTR) and after pA (pa) site locations relative to the μDys expression cassette.

It is apparent that the fusion constructs of the invention generally over-expressed miR-29c in the infected human iPS-derived cardiomyocytes by a factor of 2 to 11 fold, compared to a control in which a similar construct was used to express only μDys (and thus only background level of endogenous miR-29c expression was present).

Specific fusion constructs used to generate the data in FIG. 13 include:

29c-19nt-μDys-3UTR: a modified miR29c in miR-155 backbone, inserted into the 3′-UTR region of the μDys expression cassette (before the polyA adenylation signal sequence).

29c-19nt-μDys-pA: the same modified miR29c coding sequence in miR-155 backbone, inserted after the polyA adenylation signal sequence of the μDys expression cassette.

μDys-29c-M30E-i2: a modified miR29c coding sequence in miR-30E backbone, inserted into the intron region of the μDys expression cassette.

μDys-29c-101-i2: a modified miR29c coding sequence in miR-101 backbone, inserted into the intron region of the μDys expression cassette.

μDys-29c-3UTR-101: a modified miR29c coding sequence in miR-101 backbone, inserted into the 3′-UTR region of the μDys expression cassette.

Meanwhile, the solo constructs expressing miR-29c generally over-expressed miR-29c in the infected human iPS-derived cardiomyocytes by a factor of 6-73 fold, compared to the same control vector that expresses only μDys.

Specific solo constructs used to generate the data in FIG. 13 include:

EF1A-29c-M30E: a modified miR29c coding sequence in the miR-30E backbone, driven by the EF1A promoter.

U6-29c-M30E: a modified miR29c coding sequence in miR-30E backbone, driven by the Pol III U6 promoter.

U6-29c-v1: a miR29c coding sequence driven by the Pol III U6 promoter.

EF1A-29c-19nt: a modified miR29c coding sequence in miR-155 backbone, driven by the EF1A promoter.

EF1A-29c-155: another modified miR29c coding sequence in miR-155 backbone, driven by the EF1A promoter.

Similar trends indicating (preferential) production of miR-29c from these constructs were also obtained when these constructs were evaluated in other in vitro cell systems, including the Mouly human healthy primary myoblasts and the mouse C2C12 immortalized myoblast line (data not shown). Insertion of miR-29c elements in μDys expression cassette does not cause significant reductions in μDys mRNA production.

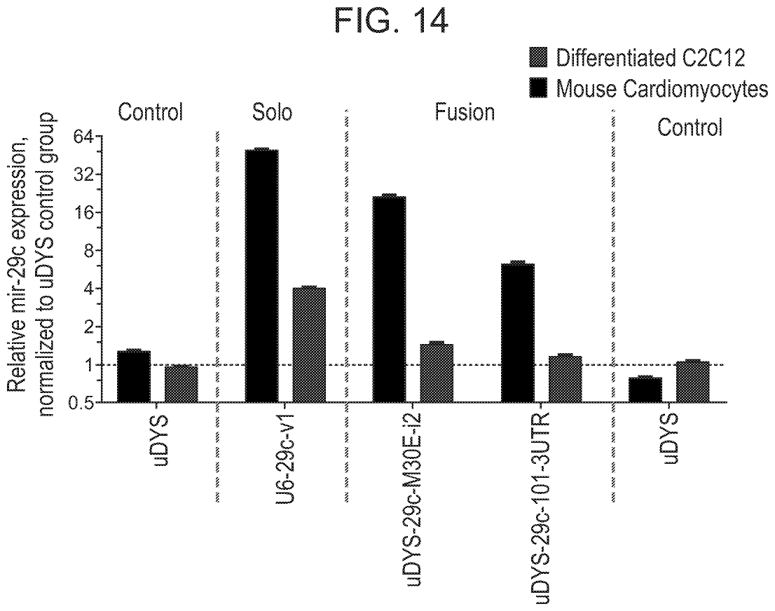

A few selected fusion recombinant viral vectors in AAV9 viral particles were also used to infect differentiated C2C12 myotube and primary mouse cardiomyocytes, and expression of miR-29c was confirmed in these cells as well. See FIG. 14, with results being expressed as relative miR-29c expression after normalization against controls expressing only μDys.

In this experiment, μDys production appeared largely unaffected relative to control group. In addition, miR-29c passenger strand levels did not show increased levels.

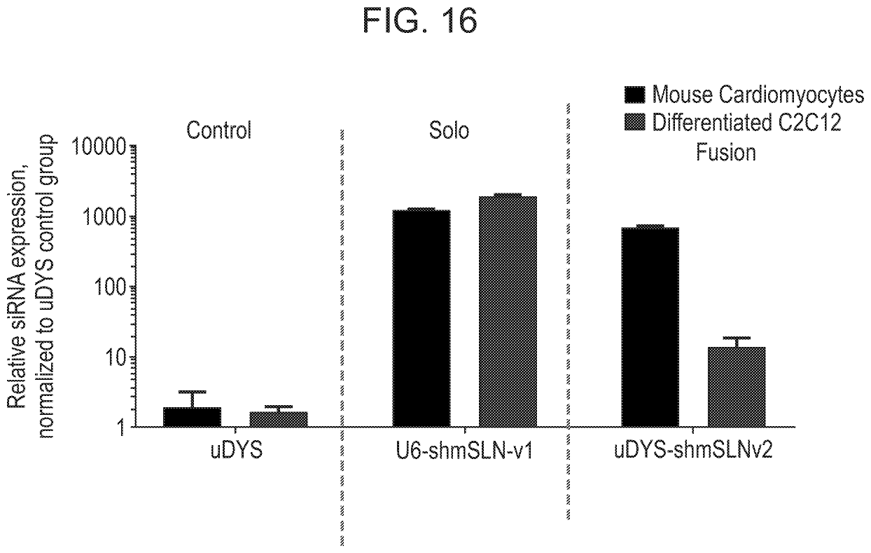

Meanwhile, expression of shSLN from the subject fusion constructs, and the resulting ˜50% down-regulation of SLN in mouse cells infected by such fusion constructs, were shown in FIGS. 16 and 15, respectively.

Specifically, three solo constructs expressing only μDys and two fusion constructs expressing μDys and shSLN were transfected to mouse C2C12 cells stably overexpressing expressing SLN-Myc/DDK (Myc-DDK-tagged SLN. DDK is the same as the trademarked FLAG® tag of Sigma Aldrich, and the Myc-DDK tag can be detected using anti-Myc or anti-DDK antibodies). The mouse C2C12 stable cell line was generated by lentiviral transduction of a vector encoding SLN-Myc/DDK and subsequent selection for stable cell lines. One of the fusion constructs “Fusion-v2” or μDys-shmSLN-v2 showed ˜50% knock-down in SLN protein expression level detected via Myc tag-specific antibodies. See FIG. 15.

The various constructs used in FIG. 15 are described below.

μDys: control AAV9 vector encoding only the μDys GOI.

EF1A-mSLN: a solo construct expressing only shRNA targeting mouse SLN. Transcription of the shRNA coding sequence is driven by the EF1A promoter.

EF1A-mSLN (V2): another solo construct expressing only shRNA targeting mouse SLN. Transcription of the shRNA coding sequence is driven by the EF1A promoter.

EF1A-mSLN (V4): yet another solo construct expressing only shRNA targeting mouse SLN. Transcription of the shRNA coding sequence is driven by the EF1A promoter.

Fusion-v1: a fusion construct of the invention that expresses both the μDys GOI and the coding sequence for the shRNA targeting mouse SLN.

Fusion-v2: another fusion construct of the invention that expresses both the μDys GOI and the coding sequence for the shRNA targeting mouse SLN.

It is apparent that the expression of mSLN was reduced by about 50% due to the infection of the mouse cell by the subject fusion construct encoding a version of mSLN shRNA.

FIG. 16 shows relative expression levels of siSLN (processed siRNA product from the transcribed shSLN) in differentiated C2C12 myotubes or mouse primary cardiomyocytes for the various recombinant AAV9 vectors encoding shSLN, either as the sole coding sequence in the viral vector (“Solo”), or as part of the fusion construct of the present disclosure (“Fusion”). siRNA production was quantified via a custom Taqman stem-loop QPCR system. The relative siSLN expression levels of the solo and fusion constructs were normalized against the level in the μDys control group, although apparent high fold changes may not be informative due to the near absent or very minimal siSLN-like RNA production in the control group. Nevertheless, it is apparent that, in both cell types tested, the solo construct expressed about 1000-fold higher level of siSLN from the strong U6 Pol III promoter, as compared to the control group. Meanwhile, the tested fusion construct expressed 1-2 magnitude higher level of siSLN compared the control.

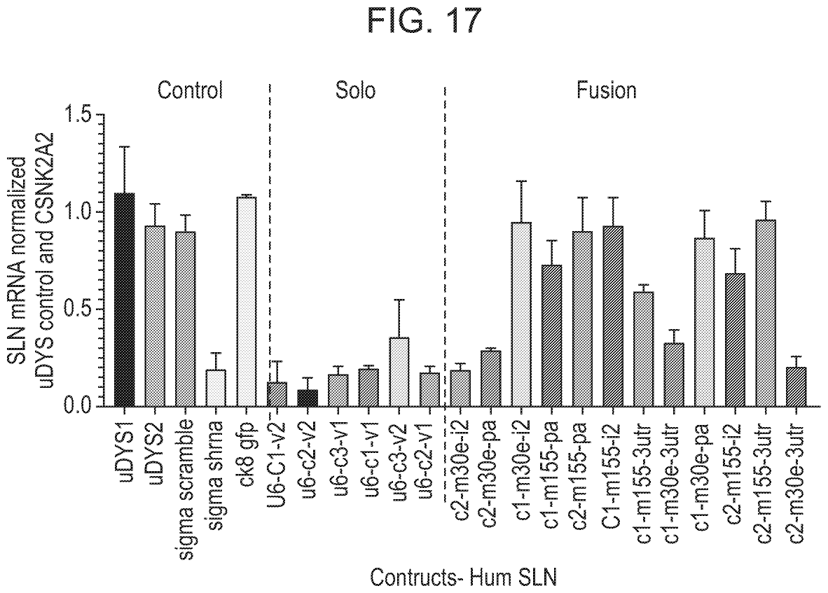

Numerous additional solo and fusion constructs expressing shRNA targeting human SLN were also tested in human iPS-derived cardiomyocytes. These include 6 solo constructs and 12 fusion constructs targeting human SLN. The fusion constructs included shSLN sequences in miR-29 and miR-155 backbones, and were inserted into the intron, 3′-UTR, or after pA sites relative to the μDys expression cassette. The results of these experiments were summarized in FIG. 17.

Specifically, several negative controls (e.g., multiple μDys and GFP plasmids) and positive controls were used in the experiments in FIG. 17. The negative controls include: two constructs (μDys1 and μDys2) expressing μDys alone (which had no effect on the expression level of SLN mRNA); a construct expressing GFP under the muscle-specific promoter CK8 (CK8-GFP) (which GFP also had no effect on SLN mRNA expression); and “sigma scramble”—a construct expressing a scrambled sequence of a hSLN-targeting shSLN (which expectedly had no effect on SLN mRNA expression). The positive control is “sigma shrna,” which is a commercially available shRNA plasmid from Sigma that encodes an hSLN-targeting shSLN that down-regulates about 80% of the hSLN mRNA.

Six solo constructs, each expressing a version of the shRNA targeting hSLN, and each under the transcriptional control of the strong Pol III U6 promoter, were tested and were shown to generally down-regulate about 80-90% of hSLN mRNA expression.

Up to 90% hSLN mRNA expression down-down were also observed across 6 solo constructs, and 4 fusion constructs of the invention. For example, the c2-m30e-i2 construct is a fusion construct that co-expresses μDys and shRNA targeting hSLN. The shRNA is embedded in the M30E backbone sequence (see above), and is inserted into the intron of the μDys expression cassette. Up to 90% of the hSLN mRNA was knocked down upon infecting the human iPS-derived cardiomyocytes with this construct.

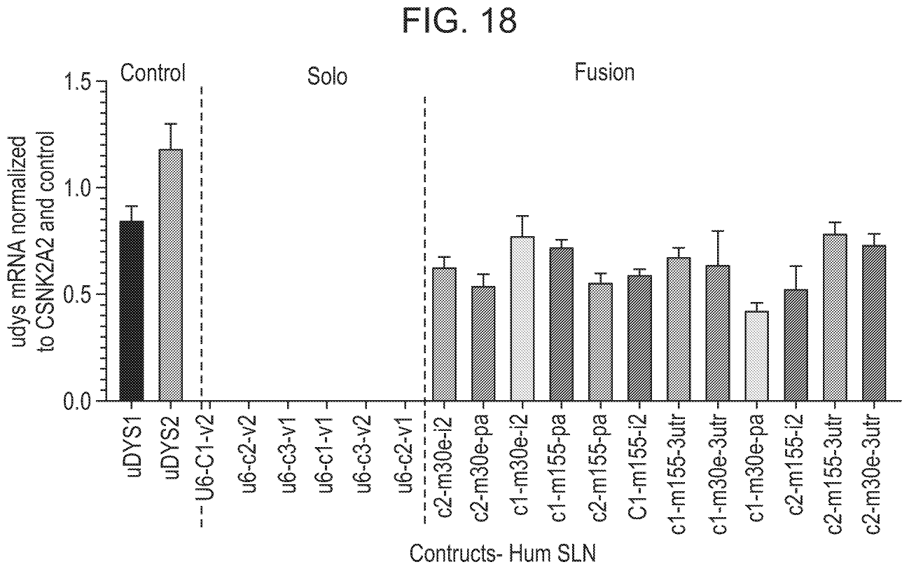

Although the fusion constructs greatly affected hSLN mRNA expression, they did not appear to have negative impact on the expression of the μDys on the same vector. As shown in FIG. 18, 6 solo and 12 fusion constructs targeting human SLN were transfected to human iPS-derived cardiomyocytes. Most fusion constructs showed largely similar (>50%)μDys mRNA expression as that of the control μDys-only constructs.

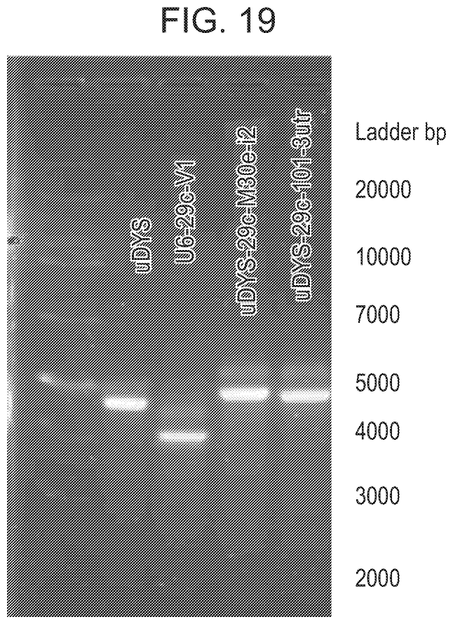

Denaturing agarose gel analysis of selected solo and two fusion constructs also confirmed that the AAV9 genomes of these miR-29c constructs were largely intact. See FIG. 19.

### Example 4 In Vivo Expression of Coding Sequences from Fusion Constructs

This experiment demonstrates that the subject fusion constructs can be used to simultaneously express μDys and one or more additional coding sequence(s) that affect a separate pathway (e.g., down-regulation of SLN, and/or up-regulation of miR-29c) to achieve better-than-solo if not synergistic therapeutic efficacy.

In this set of experiments, fusing constructs of AAV9 encoding a μDys gene as well as a second coding sequence—either miR-29c or shSLN targeting mouse SLN. Various fusion constructs were injected into 6-weeks-old male mdx mice via tail vein, at a dose of about 5E13 vg/kg (except for one group, U6-29c-v1, at 1E14 vg/kg). Expression of μDys, miR-29c, and SLN mRNA were then monitored over a period of 28 days post injection. The detailed experimental set-ups are summarized below:

| Group | Type | Name | Animal Number |
|---|---|---|---|
| miR | μDys | μDys | 4 |
| miR | Solo | U6-29c-v1 | 4 |
| miR | Fusion | μDys-29c-M30E-i2 | 4 |
| miR | Fusion | μDys-29c-101-3UTR | 4 |
| miR | Solo-1E14 | U6-29c0v1 (2X) | 2 |
| miR | Control | Control | 4 |
| shSLN | μDys | μDys | 4 |
| shSLN | Solo | U6-shmSLN-v1 | 4 |
| shSLN | Fusion | μDys-shmSLN-v2 | 4 |

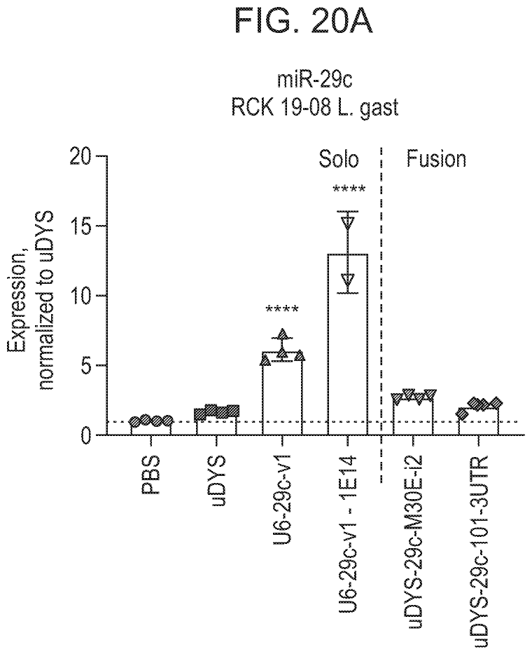

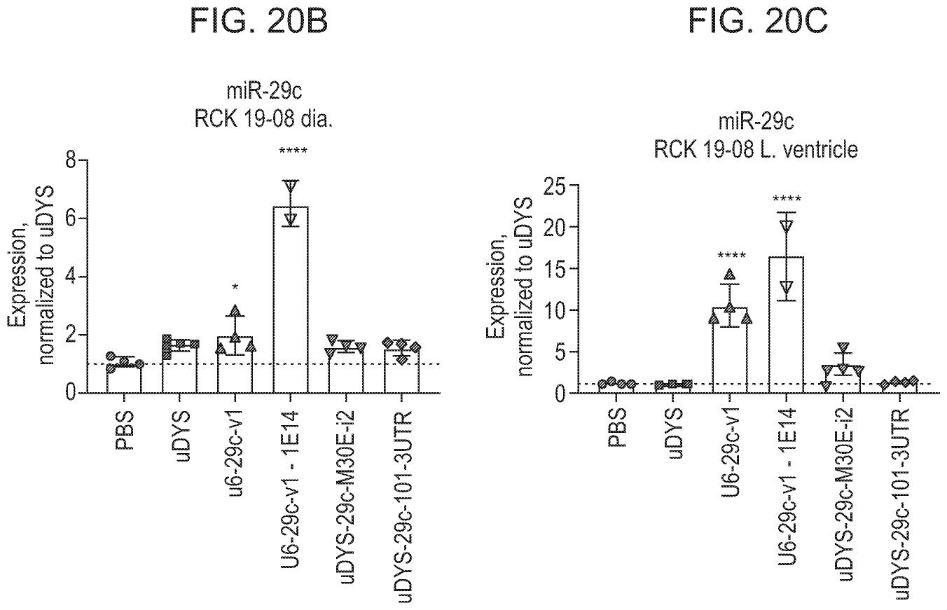

In the miR-29c experimental group, it was found that the two tested fusion constructs, one in M30E backbone and inserted into the intron of the μDys expression cassette, and one in miR-101 backbone and inserted into the 3′-UTR region of the μDys expression cassette, led to 1.4-2.8-fold miR-29c up-regulation in left gastrocnemius (FIG. 20A), diaphragm (FIG. 20B), and left ventricle (FIG. 20C). The miR-29c-μDys fusion AAV9 constructs were administered at 5E13 vg/kg dose. The solo U6 promoter-driven miR-29c construct in AAV9 produced 2-11 fold up-regulation at 5E13 vg/kg dose, and 6-16-fold at 1E14 vg/kg dose.

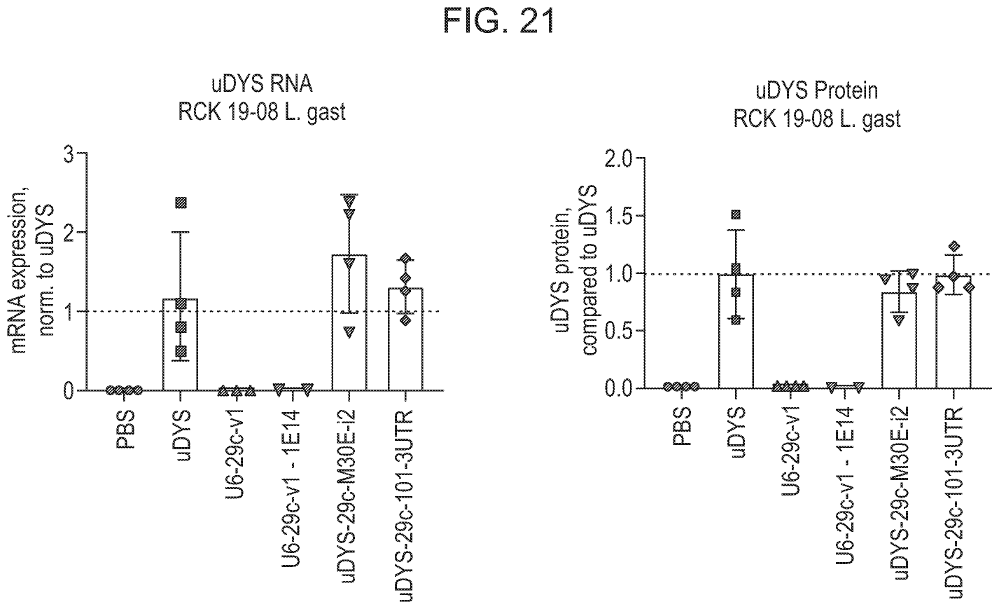

Meanwhile, miR-29c up-regulation by the fusion AAV9 constructs did not result in reduction of μDys production in gastrocnemius (FIG. 21), diaphragm (data not shown) and left ventricle (data not shown). The fusion AAV9 constructs showed similar μDys expression, at both RNA and protein levels, as that of the control μDys-only AAV9 constructs. Solo constructs expressing only miR-29c do not produce μDys, therefore showed absent μDys levels.

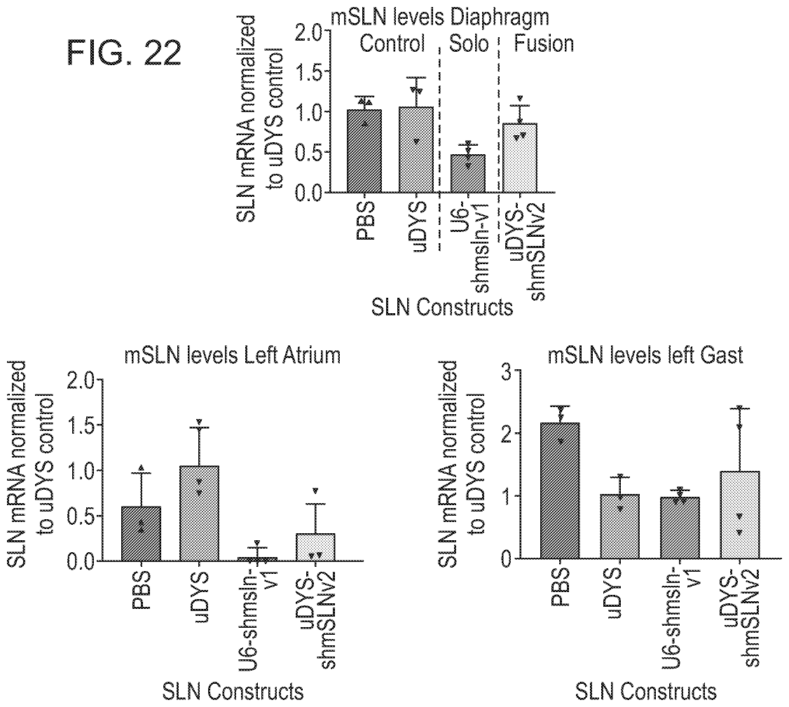

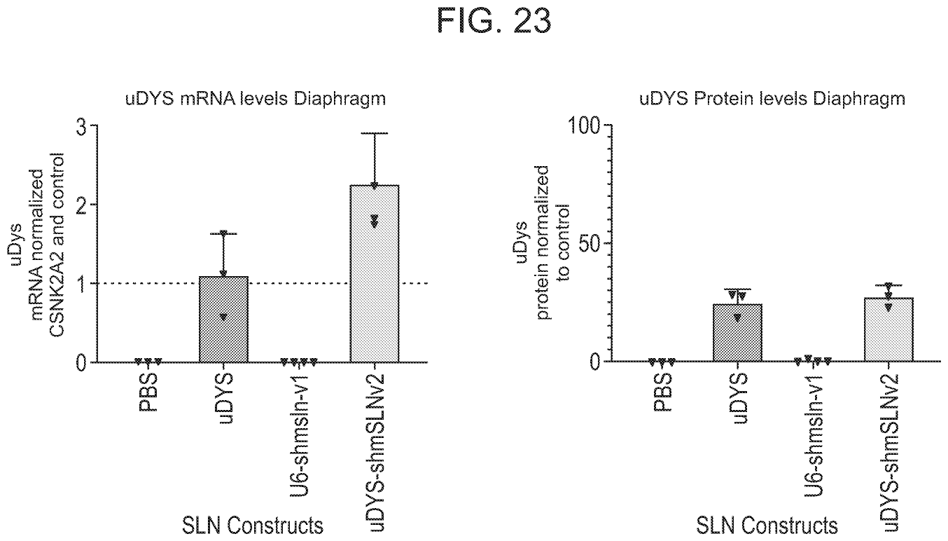

In the shSLN experimental group, it was found that the tested shSLN fusion AAV9 construct led to up to 50% mSLN mRNA down-regulation in the diaphragm, left gast, and atrium (FIG. 22), as well as in tongue (data not shown). Similarly, mSLN mRNA down-regulation by the fusion AAV9 construct did not result in reduction of μDys production, at both RNA and protein levels, in gastrocnemius (FIG. 23), diaphragm (data not shown), and left ventricle (data not shown), as compared to that of the control AAV9 expressing only μDys. Solo construct expressing only shmSLN did not produce μDys, thus showing absent μDys levels. Diaphragm results are shown. Similar results in tongue and atrium.

These data show that the subject fusion constructs can simultaneously express both the μDys gene and at least one additional coding sequence such as miR-29c or shRNA against SLN, thus achieving better therapeutic outcome compared to viral vectors expressing only one coding sequence such as μDys.

### Example 5 Coding Sequences Expressed In Vivo from the Fusion Constructs are Biologically Active

This experiment demonstrates that the coding sequences expressed from the fusion constructs of the invention are biologically active.

Dystrophin provides structural stability to the muscle cell membrane, and increased permeability of the sarcolemma leads to the release of creatine kinase (CK) from muscle fibers. Thus, increased creatine kinase (CK) levels are a hallmark of muscle damage. In DMD patients, CK levels are significantly increased above the normal range (e.g., 10-100 times the normal level since birth). Likewise, serum CK levels are considered as a general measure of muscle health in the mdx mouse model.

The data in this experiment shows that miR-29c solo (administered at the high dose of 1E14 vg/kg) and miR-29c-μDys fusion (administered at 5E13 vg/kg dose) constructs of AAV9 both reduced serum CK levels in the mdx mouse model, to the similar extent compared to the μDys control, therefore suggesting a therapeutic benefit of expressing miR-29c in DMD patients.

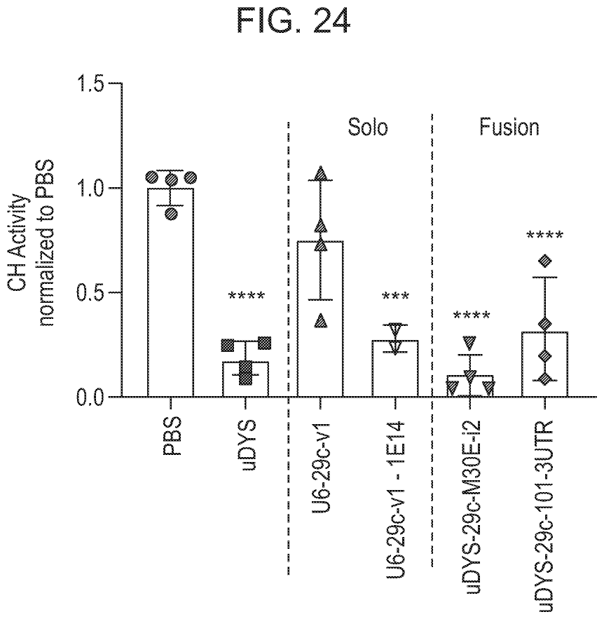

Specifically, in the in vivo experiments of Example 4, serum CK levels were also determined for the various groups of mice. FIG. 24 shows that expression of μDys alone caused significant drop in serum CK level. Co-expressing μDys and miR-29c, with both tested fusion constructs, also led to similarly significant drop in serum CK levels. Interestingly, expressing miR-29c alone also led to significant decrease of serum CK level, especially when a higher viral dose (of miR-29c-expressing solo constructs) was used.

On the other hand, tissue inhibitors of metalloproteinase-1 (TIMP-1) has been proposed as a serum biomarker for monitoring disease progression and/or treatment effects in Duchenne muscular dystrophy (DMD) patients, since serum levels of TIMP-1 were significantly higher in DMD patients compared to healthy controls. Similarly, TIMP1 is also a serum marker for muscle health in the mdx mouse model.

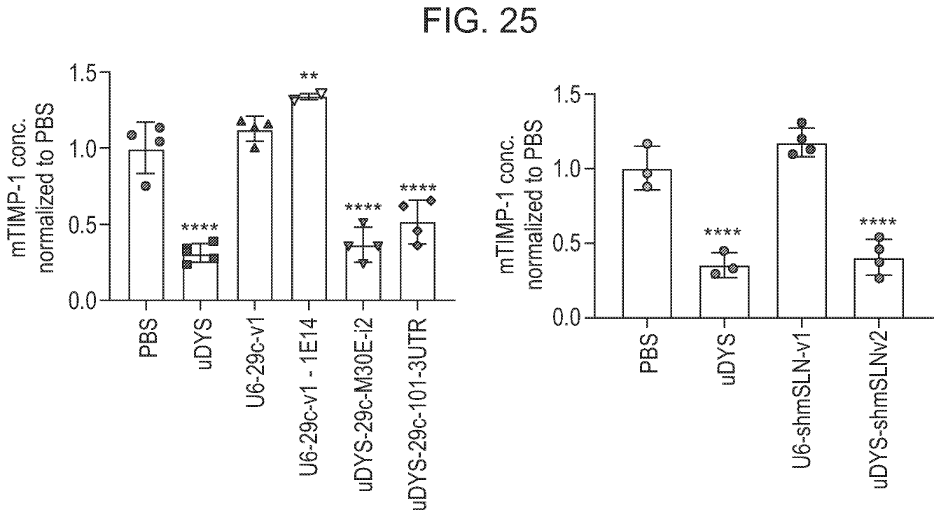

Thus in the in vivo experiments of Example 4, serum TIMP1 levels were also determined for the various groups of mdx mice. FIG. 25, left panel, shows that expression of μDys alone caused significant drop in serum TIMP1 level. Co-expressing μDys and miR-29c, with both tested fusion constructs, also led to similarly significant drop in serum TIMP1 levels. Meanwhile, expressing miR-29c alone did not lead to decrease of serum TIMP1 level, even when a higher viral dose (of miR-29c-expressing solo constructs) was used.

Likewise, FIG. 25, right panel, shows that expression of μDys alone caused significant drop in serum TIMP1 level. Co-expressing μDys and shRNA against mSLN with the tested fusion construct also led to similarly significant drop in serum TIMP1 levels. Meanwhile, expressing shRNA against mSLN alone did not lead to decrease of serum TIMP1 level.

These data suggest that the coding sequences expressed in vivo from the fusion constructs of the invention are biologically active.

### Example 6 the Fusion Constructs do not Change Biodistribution of the Viral Vectors

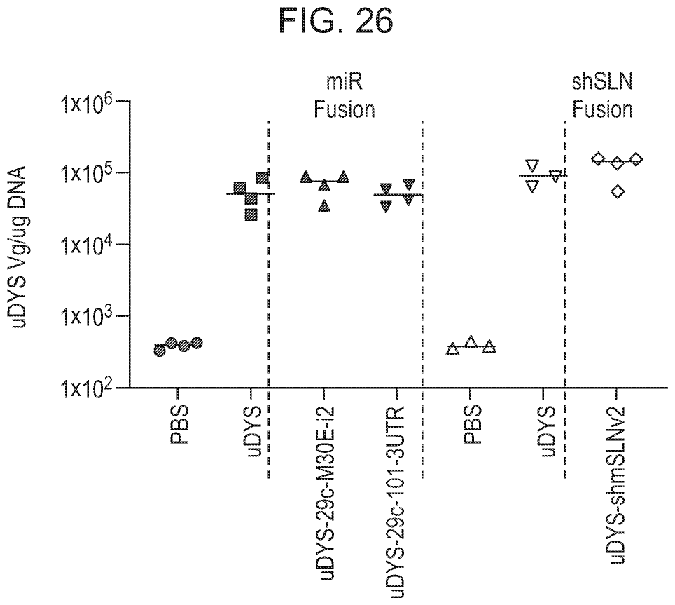

In the in vivo experiments in Example 4, biodistribution of the fusion viral vectors was compared to that of the solo viral vector expressing only μDys. It was found that biodistribution of all viral vectors used were largely identical in gastrocnemius, regardless of whether the fusion construct expresses miR-29c or shSLN. See FIG. 26.

Quantification of viral titers in gastrocnemius also showed similar viral titers with fusion and control AAV9s.

### Example 7 the Fusion Constructs do not Change Liver Biodistribution of the Viral Vectors

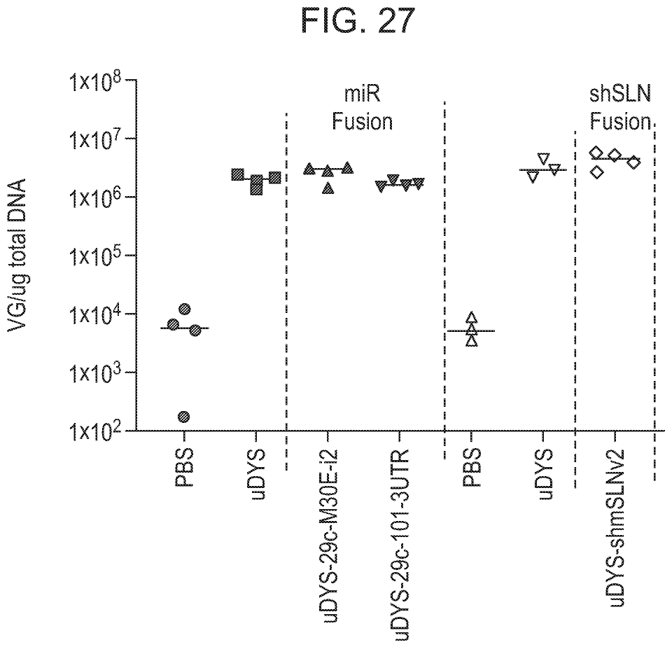

In the in vivo experiments in Example 4, liver levels of the fusion viral vectors were compared to that of the solo viral vector expressing only μDys. It was found that viral titers of all viral vectors used were largely identical in liver, regardless of whether the fusion construct expresses miR-29c or shSLN. See FIG. 27.

### Example 8 Enhanced Therapeutic Efficacy Using the Fusion Constructs Compared to μDys Single Therapy

In order to determine whether co-expressing μDys and miR-29c leads to better therapeutic efficacy and/or less complication such as fibrosis, the expression levels of two fibrotic marker genes, Col3a1 and Fn1, were examined in mice administered with the various fusion, solo, or control constructs in Example 4. Col3A1 expression and FN1 expression have been used as markers of fibrotic activity.

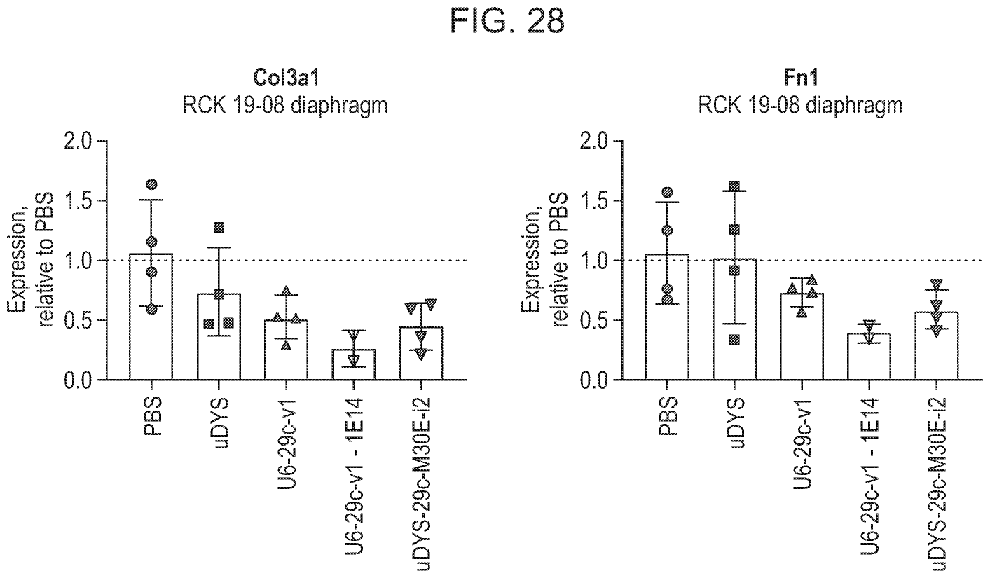

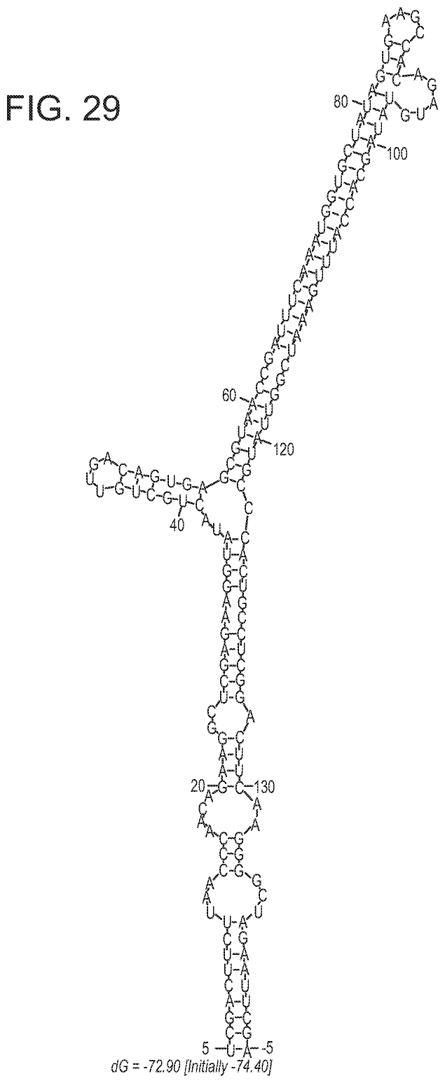

Solo AAV9 vector expressing only miR-29c resulted in decrease of Col3A1 and FN1 expression at the low dose of 5E13 vg/kg, and the high dose of 1E14 vg/kg. Fusion AAV9 vector also resulted in decrease in marker genes expressions in the diaphragm. See FIG. 28.

The results show added benefit of the fusion constructs of the invention over μDys construct alone in diaphragm, based on their effects on these two fibrotic marker genes.

### Example 9 Delivery of Enzyme-Based Gene Editing: CRISPR/Cas and sgRNA/crRNA

The subject viral vectors, e.g., rAAV viral vectors can be used to deliver CRISPR/Cas9 or CRISPR/Cas12a (or other engineered or modified Cas enzymes or homologous thereof) into a target cell, together with one or more sgRNA (for Cas9), or one or more crRNA (for Cas12a), for simultaneous knock down of target genes in the target cell. The target cell tropism can be controlled in part by the tropism of the viral particles in which the CRISPR/Cas and sgRNA/crRNA-encoding sequences resides.

For example, for AAV-mediated delivery, the GOI in the subject viral vector can be the coding sequence for CRISPR/Cas9 or CRISPR/Cas12a. The one or more sgRNA or crRNA that can be loaded onto Cas9 or Cas12a, respectively, can be expressed from the intron, 3′-UTR, or elsewhere in the expression cassette of Cas9/Cas12a.

Upon infecting the target cell with the subject viral vectors, e.g., AAV vectors, Cas proteins and sgRNA/crRNA are co-expressed inside the target cell to mediate gene editing.

1. A recombinant viral vector comprising:
a) a polynucleotide encoding a functional gene or protein of interest (GOI), such as one effective to treat a muscular dystrophy, wherein said polynucleotide comprises a 3′-UTR coding region, and is immediately 3′ to a heterologous intron sequence that enhances expression of the functional protein encoded by the polynucleotide;
b) a control element (e.g., a muscle-specific control element) operably linked to and drives the expression of the polynucleotide; and,
c) one or more coding sequences inserted in the intron sequence or in the 3′-UTR coding region;
wherein said one or more coding sequences independently encode: an RNAi sequence (siRNA, shRNA, miRNA), an antisense sequence, a guide sequence for a gene editing enzyme, a microRNA (miRNA), and/or a miRNA inhibitor.

2. The recombinant viral vector of claim 1, wherein the recombinant viral vector is a recombinant AAV (adeno associated viral) vector.

3. The recombinant viral vector of claim 1 or 2, wherein: said one or more coding sequences are inserted in the 3′-UTR coding region, or after the polyadenylation (polyA) signal sequence (e.g., AATAAA).

4. The recombinant viral vector of any one of claims 1-3, wherein expression of the functional GOI is substantially unaffected in the presence of the one or more coding sequences (e.g., as compared to otherwise identical control constructs without inserted said one or more coding sequences).

5. The recombinant viral vector of any one of claims 1-4, wherein:
a) the polynucleotide is a dystrophin microgene or minigene encoding a functional dystrophin protein; and/or,
b) the control element is a muscle-specific promoter operably linked to and drives the expression of the dystrophin minigene.

6. The recombinant viral vector of claim 5, wherein the functional dystrophin protein is microD5, and/or the muscle-specific promoter is CK promoter.

7. The recombinant viral vector of any one of claims 1-6, wherein said one or more coding sequences comprise an exon-skipping antisense sequence that induces skipping of an exon of a defective dystrophin, such as any one of exons 45-55 of dystrophin, or exon 44, 45, 51, and/or 53 of dystrophin.

8. The recombinant viral vector of any one of claims 1-7, wherein said microRNA is miR-1, miR-133a, miR-29c, miR-30c, and/or miR-206.

9. The recombinant viral vector of claim 8, wherein said microRNA is miR-29c, optionally having a modified flanking backbone sequence that enhances the processing of the guide strand of miR-29c designed for a target sequence.

10. The recombinant viral vector of claim 9, wherein said modified flanking backbone sequence is from or based on miR-30, -101, -155, or -451.

11. The recombinant viral vector of any one of claims 8-10, wherein expression of said microRNA in a host cell is up-regulated by at least about 1.5-15 fold (e.g., about 2-10 fold, about 1.4-2.8 fold, about 2-5 fold, about 5-10 fold, about 2, 3, 4, 5, 6, 7, 8, 9, 10, 11, 12, 13, 14, or about 15 fold) compared to endogenous expression of said microRNA in said host cell.

12. The recombinant viral vector of any one of claims 1-11, wherein said RNAi sequence is an shRNA against sarcolipin (shSLN).

13. The recombinant viral vector of any one of claims 1-12, wherein said one or more coding sequences encode one or more identical or different shRNAs against sarcolipin (shSLN).

14. The recombinant viral vector of claim 12 or 13, wherein said shRNA reduces sarcolipin mRNA and/or sarcolipin protein expression by at least about 50%.

15. The recombinant viral vector of any one of claims 1-14, wherein said GOI is CRISPR/Cas9, and said guide sequence is an sgRNA (single guide RNA); or wherein said GOI is CRISPR/Cas12a, and said guide sequence is a crRNA.

16. The recombinant viral vector of any one of claims 1-15, wherein said RNAi sequence (siRNA, shRNA, miRNA), said antisense sequence, said CRISPR/Cas9 sgRNA, said CRISPR/Cas12a crRNA and/or said microRNA antagonizes the function of one or more target genes, such as an inflammatory gene, an activator of NF-κB signaling pathway (e.g., TNF-α, IL-1, IL-1β, IL-6, Receptor activator of NF-κB (RANK), and Toll-like receptors (TLRs)), NF-κB, a downstream inflammatory cytokine induced by NF-κB, a histone deacetylase (e.g., HDAC2), TGF-β, connective tissue growth factor (CTGF), ollagens, elastin, a structural component of the extracellular matrix, Glucose-6-phosphate dehydrogenase (G6PD), myostatin, phosphodiesterase-5 (PED-5) or ACE, VEGF decoy-receptor type 1 (VEGFR-1 or Flt-1), and hematopoietic prostaglandin D synthase (HPGDS).

17. The recombinant viral vector of claim 1, wherein:
a) the polynucleotide encodes a functional fukutin (FKTN) protein; and/or,
b) the one or more coding sequences encode an exon-skipping antisense sequence that restores correct exon 10 splicing in a defective FKTN gene in a Fukuyama congenital muscular dystrophy (FCMD) patient.

18. The recombinant viral vector of claim 1, wherein:
a) the polynucleotide encodes a functional LAMA2 protein; and/or,
b) the one or more coding sequences encode an exon-skipping antisense sequence that restores expression of the C-terminal G-domain (exons 45-64), particularly G4 and G5 of a defective LAMA2 gene in a Merosin-deficient congenital muscular dystrophy type 1A (MDC1A) patient.

19. The recombinant viral vector of claim 1, wherein:
a) the polynucleotide encodes a functional DMPK protein, or a CLCN1 gene; and/or,
b) the RNAi sequence (siRNA, shRNA, miRNA), the antisense sequence, or the microRNA (miRNA) targets expanded repeats of mutant transcripts in a defective DMPK gene, or encodes an exon-skipping antisense sequence leading to the skipping of exon 7A in CLCN1 gene in a DM1 patient.

20. The recombinant viral vector of claim 1, wherein:
a) the polynucleotide encodes a functional DYSF protein; and/or,
b) one or more coding sequences encode an exon-skipping antisense sequence leading to the skipping of exon 32 in a defective DYSF gene in a dysferlinopathy (LGMD2B or MM) patient.

21. The recombinant viral vector of claim 1, wherein:
a) the polynucleotide encodes a functional SGCG protein; and/or,
b) one or more coding sequences encode an exon-skipping antisense sequence leading to the skipping of exons 4-7 in a defective LGMD2C gene (e.g., one with the Δ-521T SGCG mutation) in a LGMD2C patient.

22. The recombinant viral vector of any one of claims 1-21, wherein the heterologous intron sequence is SEQ ID NO: 1.

23. The recombinant viral vector of any one of claims 1-22, wherein said one or more coding sequences are inserted in the intron sequence.

24. The recombinant viral vector of any one of claims 1-23, wherein expression of the functional protein is not negatively affected by the insertion of said one or more coding sequences.

25. The recombinant viral vector of any one of claims 1-24, wherein the vector is a recombinant AAV vector of the serotype AAV1, AAV2, AAV4, AAV5, AAV6, AAV7, AAVrh74, AAV8, AAV9, AAV10, AAV 11, AAV 12, or AAV 13.

26. The recombinant viral vector of any one of claims 1-25, wherein the control element is human skeletal actin gene element, cardiac actin gene element, myocyte-specific enhancer binding factor mef, muscle creatine kinase (MCK), truncated MCK (tMCK), myosin heavy chain (MHC), C5-12, murine creatine kinase enhancer element, skeletal fast-twitch troponin c gene element, slow-twitch cardiac troponin c gene element, slow-twitch troponin i gene element, hypoxia-inducible nuclear factors, steroid-inducible element, or glucocorticoid response element (gre).

27. The recombinant viral vector of any one of claims 1-26, wherein the control element comprises the nucleotide sequence of SEQ ID NO: 10 or SEQ ID NO: 11 of WO2017/181015.

28. A composition comprising the recombinant viral vector of any one of claims 1-27.

29. The composition of claim 28, which is a pharmaceutical composition further comprising a therapeutically compatible carrier, diluent, or excipient.

30. The composition of claim 29, wherein the therapeutically acceptable carrier, diluent, or excipient is a sterile aqueous solution comprising 10 mM L-histidine at pH 6.0, 150 mM sodium chloride, and 1 mM magnesium chloride.

31. The composition of claim 29 or 30, in a dosage form of about 10 mL of aqueous solution having at least 1.6×10¹³ vector genomes.

32. The composition of any one of claims 29-31, having a potency of at least 2×10¹² vector genomes per milliliter.

33. A method of producing the composition of any one of claims 28-32, comprising producing the recombinant viral vector (e.g., the recombinant AAV vector) in a cell and lysing the cell to obtain the vector.

34. The method of claim 33, wherein the vector is a recombinant AAV vector of the serotype AAV1, AAV2, AAV4, AAV5, AAV6, AAV7, AAVrh74, AAV8, AAV9, AAV10, AAV 11, AAV 12, or AAV 13.

35. A method of treating a muscular dystrophy or dystrophinopathy in a subject in need thereof, the method comprising administering to the subject a therapeutically effective amount of the recombinant viral vector (e.g., the recombinant AAV vector) of any one of claims 1-27, or the composition of any one of claims 28-32.

36. The method of claim 35, wherein the recombinant AAV vector or the composition is administered by intramuscular injection, intravenous injection, parental administration or systemic administration.

37. The method of claim 35 or 36, wherein the muscular dystrophy is Duchenne muscular dystrophy, Becker muscular dystrophy, Fukuyama congenital muscular dystrophy (FCMD), dysferlinopathy, myotonic dystrophy, and merosin-deficient congenital muscular dystrophy type 1A, facioscapulohumeral muscular dystrophy (FSHD), congenital muscular dystrophy (CMD), or limb-girdle muscular dystrophy (LGMDR5 or LGMD2C).
